(19) United States
(12) Patent Application Publication
(10) Pub. No.: US 2026/0306465 A1
(43) Pub. Date: Oct. 8, 2026

(54) COMPOUNDS FOR TREATING HEREDITARY SPASTIC PARAPLEGIA

(71) Applicant: The Children's Medical Center Corporation, Boston, MA (US)

(72) Inventors: Darius Ebrahimi-Fakhari, Brookline, MA (US); Mustafa Sahin, Needham, MA (US); Jed L. Hubbs, Boston, MA (US); Masato Satoh, Takasaki-shi (JP)

(73) Assignee: The Children's Medical Center Corporation, Boston, MA (US)

(21) Appl. No.: 19/485,387

(22) PCT Filed: May 17, 2024
(86) PCT No.: PCT/US2024/029856

§ 371 Date: Nov. 18, 2025

Related U.S. Application Data
(60) Provisional application No. 63/503,262, filed on May 19, 2023.

(51) Int. Cl. A61K 31/437; A61P 25/00; C07D 471/04

(52) U.S. Cl. CPC A61K 31/437; A61P 25/00; C07D 471/04

(57) ABSTRACT

Provided herein are compounds that modulate Autophagy Related 9 A (ATG9A) trafficking and/or increase autophagic flux. Also provided are pharmaceutical compositions comprising the compounds, and methods of treating neurological disease or disorder, such as hereditary spastic paraplegia (HSP).

### RELATED APPLICATIONS

This application claims priority under 35 U.S.C. § 119(e) to U.S. Provisional Application, U.S. Ser. No. 63/503,262, filed May 19, 2023, which is incorporated herein by reference in its entirety.

### FEDERALLY SPONSORED RESEARCH

This invention was made with government support under Grant Number NS123552, awarded by the National Institutes of Health. The Government has certain rights in the invention.

### BACKGROUND

Despite remarkable advances in the ability to delineate the genetic causes of rare neurological diseases, it is estimated that specific therapies exist for less than 5%. Thus, there is a significant unmet need for developing and implementing novel platforms for drug discovery. Informed by disease-relevant cellular phenotypes, automated, unbiased cell-based high-throughput small molecule screens have the potential to uncover novel therapeutic targets.

Adaptor protein complex 4 (AP-4)-related hereditary spastic paraplegia (AP-4-HSP, which includes AP4B1-associated SPG47 (OMIM #614066), AP4M1-associated SPG50 (OMIM #612936), AP4E1-associated SPG51 (OMIM #613744) and AP4S1-associated SPG52 (OMIM #614067), is a rare but prototypical form of childhood-onset complex hereditary spastic paraplegia (HSP) and an important genetic mimic of cerebral palsy. Children with AP-4-HSP present with features of both a neurodevelopmental disorder (e.g., early-onset global developmental delay and seizures, microcephaly, and developmental brain malformations) and a neurodegenerative disease (e.g., progressive spasticity and weakness, loss of ambulation, and extrapyramidal movement disorders). AP-4-HSP is caused by bi-allelic loss-of-function variants in any of the four AP-4 subunits (ε, β4, μ4, σ4), leading to impaired AP-4 assembly and function. AP-4 is an obligate heterotetrameric protein complex that mediates transport from the trans-Golgi network (TGN) to the cell periphery, including sites of autophagosome biogenesis. The core autophagy protein and lipid scramblase ATG9A has been identified as a major cargo of AP-4, linking loss of AP-4 function to defective autophagy. AP-4 deficiency in non-neuronal and neuronal cells leads to an accumulation of ATG9A in the TGN, including in iPSC-derived neurons from AP-4-HSP patients. From this and overlapping neuronal phenotypes of AP-4 and Atg9a knockout mice, the following working model for AP-4 deficiency emerges: (1) AP-4 is required for trafficking of ATG9A from the TGN; (2) loss-of-function variants in AP-4 subunits lead to a loss of AP-4 function; (3) ATG9A accumulates in the TGN leading to a reduction of axonal delivery of ATG9A; (4) lack of ATG9A at the distal axon impairs autophagy leading to axonal degeneration. Other AP-4 cargo proteins identified to date include the poorly characterized transmembrane proteins SERINC1 and SERINC3, and the endocannabinoid producing enzyme DAG lipase beta (DAGLB).

### SUMMARY

The present disclosure stems from the recognition that unbiased phenotypic screens in patient-relevant disease models offer potential to identify novel therapeutic targets for rare diseases. As described herein, a high-throughput screening assay was designed and employed to identify molecules that correct aberrant protein trafficking in adaptor protein complex 4 (AP-4)-deficiency, a rare but prototypical form of childhood-onset hereditary spastic paraplegia characterized by deficient trafficking of the autophagy protein ATG9A.

As such, compounds of the disclosure restore ATG9A pathology in multiple disease models, including patient-derived fibroblasts and iPSC-derived neurons. Moreover, multiparametric orthogonal strategies and integrated transcriptomic and proteomic approaches identify putative molecular targets of the disclosed compounds and their mechanisms of action. Molecular regulators of intracellular ATG9A trafficking are also identified.

Accordingly, in one aspect, provided herein are compounds of Formula (I):

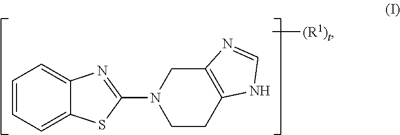

and pharmaceutically acceptable salts, solvates, hydrates, polymorphs, co-crystals, tautomers, stereoisomers, isotopically labeled derivatives, and prodrugs thereof, wherein: each occurrence of R¹ is, independently, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —C(═O)NR^{A}S(O)₂R^{A}, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, OS(O)₂R^{A}S(O)₂NR^{A}C(O)R^{A}, —S(O)₂N(R^{A})₂, —S(O)₂OR^{A} or —S(O)₂R^{A}; or two R¹ groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0 or a positive integer; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
wherein, when present, each occurrence of R¹ is bound to any substitutable atom of the compound.

In another aspect disclosed are pharmaceutical compositions comprising a compound of Formula (I), or a pharmaceutically acceptable salt, solvate, hydrate, polymorph, co-crystal, tautomer, stereoisomer, isotopically labeled derivative, or prodrug thereof, and a pharmaceutically acceptable excipient.

In another aspect disclosed are kits comprising a compound of Formula (I), or a pharmaceutically acceptable salt, solvate, hydrate, polymorph, co-crystal, tautomer, stereoisomer, isotopically labeled derivative, or prodrug thereof, or a pharmaceutical composition of the disclosure, and instructions for administering the compound or pharmaceutical composition to a subject in need thereof.

In another aspect disclosed are methods of treating a neurological disease or disorder, the methods comprising administering an effective amount of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition of the disclosure, to a subject in need thereof.

In another aspect disclosed are methods of modulating Autophagy Related 9A (ATG9A) trafficking in or from a cell, the methods comprising contacting an effective amount of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition of the disclosure, with the cell.

In another aspect disclosed are methods of modulating intracellular vesicle trafficking and increasing autophagic flux in a cell, the methods comprising contacting an effective amount of a compound of Formula (I), or a pharmaceutically acceptable salt, solvate, hydrate, polymorph, co-crystal, tautomer, stereoisomer, isotopically labeled derivative, or prodrug thereof, or a pharmaceutical composition of the disclosure, with the cell.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups.

Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In certain embodiments, an alkyl group has 1 to 9 carbon atoms (“C₁₋₉ alkyl”). In certain embodiments, an alkyl group has 1 to 8 carbon atoms (“C₁₋₈ alkyl”). In certain embodiments, an alkyl group has 1 to 7 carbon atoms (“C₁₋₇ alkyl”). In certain embodiments, an alkyl group has 1 to 6 carbon atoms (“C₁₋₆ alkyl”). In certain embodiments, an alkyl group has 1 to 5 carbon atoms (“C₁₋₅ alkyl”). In certain embodiments, an alkyl group has 1 to 4 carbon atoms (“C₁₋₄ alkyl”). In certain embodiments, an alkyl group has 1 to 3 carbon atoms (“C₁₋₃ alkyl”). In certain embodiments, an alkyl group has 1 to 2 carbon atoms (“C₁₋₂ alkyl”). In certain embodiments, an alkyl group has 1 carbon atom (“C₁ alkyl”). In certain embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In certain embodiments, the haloalkyl moiety has 1 to 8 carbon atoms (“C₁₋₈ haloalkyl”). In certain embodiments, the haloalkyl moiety has 1 to 6 carbon atoms (“C₁₋₆ haloalkyl”). In certain embodiments, the haloalkyl moiety has 1 to 4 carbon atoms (“C₁₋₄ haloalkyl”). In certain embodiments, the haloalkyl moiety has 1 to 3 carbon atoms (“C₁₋₃ haloalkyl”). In certain embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In certain embodiments, a heteroalkyl group is a saturated group having 1 to 9 carbon atoms and 1 or more heteroatoms within the parent chain (“heteroC₁₋₉ alkyl”). In certain embodiments, a heteroalkyl group is a saturated group having 1 to 8 carbon atoms and 1 or more heteroatoms within the parent chain (“heteroC₁₋₈ alkyl”). In certain embodiments, a heteroalkyl group is a saturated group having 1 to 7 carbon atoms and 1 or more heteroatoms within the parent chain (“heteroC₁₋₇ alkyl”). In certain embodiments, a heteroalkyl group is a saturated group having 1 to 6 carbon atoms and 1 or more heteroatoms within the parent chain (“heteroC₁₋₆ alkyl”). In certain embodiments, a heteroalkyl group is a saturated group having 1 to 5 carbon atoms and 1 or 2 heteroatoms within the parent chain (“heteroC₁₋₅ alkyl”). In certain embodiments, a heteroalkyl group is a saturated group having 1 to 4 carbon atoms and lor 2 heteroatoms within the parent chain (“heteroC₁₋₄ alkyl”). In certain embodiments, a heteroalkyl group is a saturated group having 1 to 3 carbon atoms and 1 heteroatom within the parent chain (“heteroC₁₋₃ alkyl”). In certain embodiments, a heteroalkyl group is a saturated group having 1 to 2 carbon atoms and 1 heteroatom within the parent chain (“heteroC₁₋₂ alkyl”). In certain embodiments, a heteroalkyl group is a saturated group having 1 carbon atom and 1 heteroatom (“heteroC₁ alkyl”). In certain embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In certain embodiments, an alkenyl group has 2 to 9 carbon atoms (“C₂₋₉ alkenyl”). In certain embodiments, an alkenyl group has 2 to 8 carbon atoms (“C₂₋₈ alkenyl”). In certain embodiments, an alkenyl group has 2 to 7 carbon atoms (“C₂₋₇ alkenyl”). In certain embodiments, an alkenyl group has 2 to 6 carbon atoms (“C₂₋₆ alkenyl”). In certain embodiments, an alkenyl group has 2 to 5 carbon atoms (“C₂₋₅ alkenyl”). In certain embodiments, an alkenyl group has 2 to 4 carbon atoms (“C₂₋₄ alkenyl”). In certain embodiments, an alkenyl group has 2 to 3 carbon atoms (“C₂₋₃ alkenyl”). In certain embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

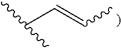

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In certain embodiments, a heteroalkenyl group has 2 to 9 carbon atoms at least one double bond, and 1 or more heteroatoms within the parent chain (“heteroC₂₋₉ alkenyl”). In certain embodiments, a heteroalkenyl group has 2 to 8 carbon atoms, at least one double bond, and 1 or more heteroatoms within the parent chain (“heteroC₂₋₈ alkenyl”). In certain embodiments, a heteroalkenyl group has 2 to 7 carbon atoms, at least one double bond, and 1 or more heteroatoms within the parent chain (“heteroC₂₋₇ alkenyl”). In certain embodiments, a heteroalkenyl group has 2 to 6 carbon atoms, at least one double bond, and 1 or more heteroatoms within the parent chain (“heteroC₂₋₆ alkenyl”). In certain embodiments, a heteroalkenyl group has 2 to 5 carbon atoms, at least one double bond, and 1 or 2 heteroatoms within the parent chain (“heteroC₂₋₅ alkenyl”). In certain embodiments, a heteroalkenyl group has 2 to 4 carbon atoms, at least one double bond, and lor 2 heteroatoms within the parent chain (“heteroC₂₋₄ alkenyl”). In certain embodiments, a heteroalkenyl group has 2 to 3 carbon atoms, at least one double bond, and 1 heteroatom within the parent chain (“heteroC₂₋₃ alkenyl”). In certain embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In certain embodiments, an alkynyl group has 2 to 9 carbon atoms (“C₂₋₉ alkynyl”). In certain embodiments, an alkynyl group has 2 to 8 carbon atoms (“C₂₋₈ alkynyl”). In certain embodiments, an alkynyl group has 2 to 7 carbon atoms (“C₂₋₇ alkynyl”). In certain embodiments, an alkynyl group has 2 to 6 carbon atoms (“C₂₋₆ alkynyl”). In certain embodiments, an alkynyl group has 2 to 5 carbon atoms (“C₂₋₅ alkynyl”). In certain embodiments, an alkynyl group has 2 to 4 carbon atoms (“C₂₋₄ alkynyl”). In certain embodiments, an alkynyl group has 2 to 3 carbon atoms (“C₂₋₃ alkynyl”). In certain embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In certain embodiments, a heteroalkynyl group has 2 to 9 carbon atoms, at least one triple bond, and 1 or more heteroatoms within the parent chain (“heteroC₂₋₉ alkynyl”). In certain embodiments, a heteroalkynyl group has 2 to 8 carbon atoms, at least one triple bond, and 1 or more heteroatoms within the parent chain (“heteroC₂₋₈ alkynyl”). In certain embodiments, a heteroalkynyl group has 2 to 7 carbon atoms, at least one triple bond, and 1 or more heteroatoms within the parent chain (“heteroC₂₋₇ alkynyl”). In certain embodiments, a heteroalkynyl group has 2 to 6 carbon atoms, at least one triple bond, and 1 or more heteroatoms within the parent chain (“heteroC₂₋₆ alkynyl”). In certain embodiments, a heteroalkynyl group has 2 to 5 carbon atoms, at least one triple bond, and 1 or 2 heteroatoms within the parent chain (“heteroC₂₋₆ alkynyl”). In certain embodiments, a heteroalkynyl group has 2 to 4 carbon atoms, at least one triple bond, and lor 2 heteroatoms within the parent chain (“heteroC₂₋₄ alkynyl”). In certain embodiments, a heteroalkynyl group has 2 to 3 carbon atoms, at least one triple bond, and 1 heteroatom within the parent chain (“heteroC₂₋₃ alkynyl”). In certain embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In certain embodiments, a carbocyclyl group has 3 to 10 ring carbon atoms (“C₃₋₁₀ carbocyclyl”). In certain embodiments, a carbocyclyl group has 3 to 8 ring carbon atoms (“C₃₋₈ carbocyclyl”). In certain embodiments, a carbocyclyl group has 3 to 7 ring carbon atoms (“C₃₋₇ carbocyclyl”). In certain embodiments, a carbocyclyl group has 3 to 6 ring carbon atoms (“C₃₋₆ carbocyclyl”). In certain embodiments, a carbocyclyl group has 4 to 6 ring carbon atoms (“C₄₋₆ carbocyclyl”). In certain embodiments, a carbocyclyl group has 5 to 6 ring carbon atoms (“C₅₋₆ carbocyclyl”). In certain embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In certain embodiments, “carbocyclyl” is a monocyclic, saturated carbocyclyl group having from 3 to 14 ring carbon atoms (“C₃₋₁₄ cycloalkyl”). In certain embodiments, a cycloalkyl group has 3 to 10 ring carbon atoms (“C₃₋₁₀ cycloalkyl”). In certain embodiments, a cycloalkyl group has 3 to 8 ring carbon atoms (“C₃₋₈ cycloalkyl”). In certain embodiments, a cycloalkyl group has 3 to 6 ring carbon atoms (“C₃₋₆ cycloalkyl”). In certain embodiments, a cycloalkyl group has 4 to 6 ring carbon atoms (“C₄₋₆ cycloalkyl”). In certain embodiments, a cycloalkyl group has 5 to 6 ring carbon atoms (“C₅₋₆ cycloalkyl”). In certain embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Carbocyclylalkyl” is a subset of “alkyl” and refers to an alkyl group substituted by a carbocyclyl group, wherein the point of attachment is on the alkyl moiety.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In certain embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In certain embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In certain embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In certain embodiments, the 5-6 membered heterocyclyl has 1-3 ring heteroatoms selected from nitrogen, oxygen, and sulfur. In certain embodiments, the 5-6 membered heterocyclyl has 1-2 ring heteroatoms selected from nitrogen, oxygen, and sulfur. In certain embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Heterocyclylalkyl” is a subset of “alkyl” and refers to an alkyl group substituted by an heterocyclyl group, wherein the point of attachment is on the alkyl moiety.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In certain embodiments, an aryl group has 6 ring carbon atoms (“C₆ aryl”; e.g., phenyl). In certain embodiments, an aryl group has 10 ring carbon atoms (“C₁₀ aryl”; e.g., naphthyl such as 1-naphthyl and 2-naphthyl). In certain embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In certain embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In certain embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In certain embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In certain embodiments, the 5-6 membered heteroaryl has 1-3 ring heteroatoms selected from nitrogen, oxygen, and sulfur. In certain embodiments, the 5-6 membered heteroaryl has 1-2 ring heteroatoms selected from nitrogen, oxygen, and sulfur. In certain embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{c}!c)₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃+X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino.

In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R′, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂—CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇-C₁₂ arylalkyl esters of the compounds described herein may be preferred.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal.

“Disease,” “disorder,” and “condition” are used interchangeably herein.

The term “administer,” “administering,” or “administration” refers to implanting, absorbing, ingesting, injecting, inhaling, or otherwise introducing a compound described herein, or a composition thereof, in or on a subject.

As used herein, and unless otherwise specified, the terms “treat,” “treating” and “treatment” contemplate an action that occurs while a subject is suffering from the specified infectious disease or inflammatory condition, which reduces the severity of the infectious disease or inflammatory condition, or retards or slows the progression of the infectious disease or inflammatory condition (“therapeutic treatment”), and also contemplates an action that occurs before a subject begins to suffer from the specified infectious disease or inflammatory condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of an infectious disease or inflammatory condition, or to delay or minimize one or more symptoms associated with the infectious disease or inflammatory condition. A therapeutically effective amount of a compound means an amount of therapeutic agent, alone or in combination with other therapies, which provides a therapeutic benefit in the treatment of the infectious disease or inflammatory condition. The term “therapeutically effective amount” can encompass an amount that improves overall therapy, reduces or avoids symptoms or causes of infectious disease or inflammatory condition, or enhances the therapeutic efficacy of another therapeutic agent.

As used herein, and unless otherwise specified, a “prophylactically effective amount” of a compound is an amount sufficient to prevent an infectious disease or inflammatory condition, or one or more symptoms associated with the infectious disease or inflammatory condition, or prevent its recurrence. A prophylactically effective amount of a compound means an amount of a therapeutic agent, alone or in combination with other agents, which provides a prophylactic benefit in the prevention of the infectious disease or inflammatory condition. The term “prophylactically effective amount” can encompass an amount that improves overall prophylaxis or enhances the prophylactic efficacy of another prophylactic agent.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1M show the establishment of a cell-based phenotypic small molecule screening platform using ATG9A translocation as a surrogate for AP-4 function and primary screening of 28,864 novel small molecule compounds. FIG. 1A shows an overview of the primary screening of 28,864 novel small molecule compounds in fibroblasts from a patient with AP-4-HSP due to biallelic loss-of-function variants in AP4B1. FIG. 1B shows an illustration of the automated image analysis pipeline. Representative images of fibroblasts from a patient with HSP-SPG47 (negative control, LoF/LoF) and their sex-matched heterozygous parent (positive control, WT/LoF) are shown. 4 markers are captured including Phalloidin, DAPI, TGN and ATG9A. The TGN and ATG9A channels are additionally depicted in greyscale. Through a series of masks, the intracellular distribution of ATG9A was calculated at the level of individuals cells, with thousands to millions of cells per experiment. Scale bar: 20 μm. FIG. 1C shows an overview of the high-throughput platform and workflow. The assay was miniaturized to 96- or 384-well microplates. Cells were stained using automated liquid handlers and imaged using an automated high-content confocal microscope, followed by automated image analysis. Primary metric is the ‘ATG9A ratio’, which was calculated by dividing the ATG9A fluorescence intensity inside the TGN by the ATG9A fluorescence intensity in the cytoplasm. FIGS. 1D-1F show the distribution of ATG9A fluorescence intensities inside (FIG. 1D) and outside (FIG. 1E) the TGN, as well as the ATG9A ratio (FIG. 1F) are shown on a per cell basis. 99,927 WT/LoF and 119,522 LoF/LoF cells were quantified. FIG. 1G shows that cell counts were measured for each experimental well. 1312 wells were analyzed per condition. FIGS. 1H and 1I show that replicate plots were generated by random sampling of the 82 plates from the primary screen in two groups. Similar positions on the assay plates were plotted against each other with respect to the ATG9A fluorescence intensity inside the TGN (FIG. 1H) and the ATG9A ratio (FIG. 1I). Replicate correlations for both analysis strategies were assessed by averaging the Pearson correlation coefficients of 100 random sampling tests. The ATG9A ratio shows a mean Pearson correlation coefficient (r) of 0.9, while the ATG9A fluorescence inside the TGN shows an average r of 0.82. FIG. 1J demonstrates the discriminative power of the ATG9A ratio in separating positive and negative controls. Statistical testing was done using the T-Test. Quantification was done using per well means. 1312 wells per condition were included. Positive and negative controls showed a robust separation (p<0.0001). FIG. 1K shows that in order to test the robustness of separation of the ATG9A ratio between positive (WT/LoF) and negative controls (LoF/LoF), a dataset containing measurement for 99,927 WT/LoF and 119,522 LoF/LoF cells was partitioned into a training set (70% of data) and a test set (30%). A generalized linear model was trained using the training set. The performance of the prediction model using the test set is shown in (FIG. 1K). The AUC was 0.96. FIG. 1L shows the impact of 28,864 compounds applied for 24 hours at a concentration 10 μM. Z-scores for the primary metric, the ATG9A ratio are shown. All data points represent per well means. The mean of the positive control (WT/LoF) is shown as a grey dotted line, indicated at “A”. The grey shaded areas at “A” represent ±1 SD. Active compounds were a priori defined as those reducing the ATG9A ratio by at least 3 SD compared to negative controls. Toxicity was defined as a reduction of cell count of at least 2SD compared to the negative control. 501 compounds show activity by reducing the ATG9A ratio by more than 3 SD. FIG. 1M shows the distribution of Z-scores of all non-toxic 27,412 compounds. Active compounds are highlighted in dark grey.

FIGS. 2A-2C show that the counter-screen in fibroblasts from AP-4-HSP patients confirms 16 compounds that lead to dose-dependent redistribution of ATG9A. FIG. 2A shows an overview of the counter-screen of the 503 active compounds identified in the primary screen. To assess for dose-dependent effects, compounds were screened in AP-4-HSP patient-derived fibroblasts in 384-well microplates using 11-point titrations ranging from 40 nM to 40 μM. All concentrations were screened in duplicates. Active compounds were a priori defined as those reducing the ATG9A ratio by at least 3SD compared to negative controls, in more than one concentration. Toxicity was defined as a reduction of the cell count of at least 2 SD compared to negative controls. 51 compounds demonstrated a clear and reproducible dose-response relationship and raised no suspicion for autofluorescence on automated and manual review. 34 compounds showed autofluorescence or resulted in imaging artifacts. One active compound was unavailable from the manufacturer and was therefore excluded from subsequent testing. FIG. 2B shows the baseline differences in the ATG9A distribution in WT/LoF (n=269) vs. LoF/LoF (n=269) fibroblasts. Statistical testing was done using a T-Test. Positive and negative controls showed a robust separation (p<0.0001). FIG. 2C shows dose-response curves that were fitted using a four-parameter logistic regression model and then ED50 concentrations were calculated. All concentrations were tested in biologic duplicates. Most ED50 were in the low micromolar range (median: 4.66 μM, IQR: 8.63). Black dashed lines represent the a priori defined thresholds of +/−3SD compared to the negative control (LoF/LoF). Triangles represent toxic concentrations based on the a priori defined threshold of a reduction of cell counts of at least 2 SD compared to the negative control. The “B” dashed line represents the mean of negative controls, while the “A” dashed line depicts the mean of the positive controls (WT/LoF). Representative images of the ED50 are shown for each active compound. Representative images show a merge of the 4 channels: Phalloidin, DAPI, TGN and ATG9A, as well as the TGN and ATG9A channels in greyscale.

FIGS. 3A-3O show that the orthogonal assays in AP4B1^{KO} SH-SY5Y cells confirm 5 active compounds. FIG. 3A shows an overview of the orthogonal screen of 16 active compounds in differentiated AP4B1^{KO} SH-SY5Y cells, a neuronal model of AP-4 deficiency. Active compounds were a priori defined as those reducing the ATG9A ratio by at least 3 SD compared to negative controls. Toxicity was defined as a reduction of cell count of at least 2 SD compared to the negative control. FIG. 3B shows the baseline differences in ATG9A ratios of AP4B1^{WT} vs. AP4B1^{KO} SH-SY5Y cells which were quantified from 160 AB4B1^{WT} and 158 AB4B1^{KO} wells from 5 assay plates. Statistical testing was performed using a T-Test. Positive and negative controls showed a robust separation (p<0.0001). FIGS. 3C-3G show dose-response curves for ATG9A ratios in AB4B1^{KO} cells treated with different compounds. Data points represent per-well means from 3 different assay plates. Dashed lines show mean Z-scores for positive (“A”) and negative (“B”) controls. Shaded areas represent ±1 SD. FIG. 3H shows representative images of the intracellular ATG9A distribution for individual compounds. The merged image shows beta-3 tubulin, DAPI, the TGN and ATG9A. The TGN and ATG9A channels are further separately depicted in greyscale. Scale bar: 10 μm. FIG. 3I shows the baseline differences of DAGLB ratios in AP4B1^{WT} vs. AP4B1^{KO} cells were quantified from 192 AB4B1^{WT} and 192 AB4B1^{KO} wells from 4 assay plates. Statistical testing was done using a T-Test. Positive and negative controls showed a robust separation (p<0.0001). FIGS. 3J-3N show the dose-response curves for DAGLB ratios in AB4B1^{KO} cells treated with different compounds. All data points represent per-well means from 4 different assay plates. Dashed lines show mean Z-scores for positive (“A”) and negative (“B”) controls. Shaded areas represent ±1 SD. FIG. 3O shows representative images of the intracellular DAGLB distribution for individual compounds. The merge shows beta-3 tubulin, DAPI, the TGN and DAGLB. The TGN and DAGLB channels are further separately depicted in greyscale. Scale bar: 10 μm.

FIGS. 4A-4G show multiparametric profiling of 5 active compounds in AP4B1^{KO} SH-SY5Y cells. FIG. 4A shows multiparametric profiling of images of 5373 cells acquired using 4 fluorescent channels. Scale: 10 μm. A total of 90 measurements per cell were generated for the cytoskeleton (beta-3 tubulin), the nucleus (DAPI), the TGN (TNG46) and ATG9A vesicles (ATG9A). The different steps of data preprocessing and phenotypic clustering using principal component analysis (PCA) are shown. FIG. 4B demonstrates that PCA shows different clusters of cells based on 85 phenotypic features. The first two principal components (PC1 and PC2) explain 43.2% of the observed variance. FIG. 4C shows a bar plot summarizing the variance explained by the first 10 principal components (PCs). Most of the variance is explained by PC1 and to a lesser degree PC2. FIG. 4D shows the correlation analysis of PC1 with all 85 features using the Pearson correlation coefficient. Grey lines, labeled “A”, represent cut-offs for correlations >0.75. FIG. 4E shows a zoom-in on selected features of interest showing a correlation with PC1>0.75. FIG. 4F shows a measurements of TGN intensity and descriptors of TGN shape and network complexity for the individual hit compounds as line graphs and FIG. 4G is summarized using heatmap visualization.

FIGS. 5A-5I show that compound C-01 restores ATG9A and DAGLB trafficking in iPSC-derived neurons from AP-4-HSP patients. FIG. 5A shows an overview of the testing of 5 active compounds in iPSC-derived cortical neurons from a patient with AP4M1-associated SPG50 compared to heterozygous controls (same-sex parent). Active compounds were defined as those reducing the ATG9A ratio by at least 3 SD compared to negative controls (patient-derived iPSC-neurons treated with vehicle). Toxicity was defined as a reduction of cell count of at least 2 SD compared to the negative control. FIG. 5B shows baseline differences of ATG9A ratios in controls vs. patient-derived iPSC-neurons were quantified using per well means of 60 wells per condition from 5 plates. Statistical testing was done using a T-Test. Positive and negative controls showed a robust separation (p<0.0001). FIG. 5C shows representative images of iPSC-neurons from a patient with SPG50 treated with individual compounds at 5 μM for 24 hours (~ED50 in prior experiments). The merge shows beta-3 tubulin, DAPI, the Golgi and ATG9A. The Golgi and ATG9A channels are further separately depicted in greyscale. Scale: 10 μm. FIGS. 5D-5F show dose-response curves for ATG9A ratios in iPSC-neurons from a patient with SPG50 treated with individual compounds for 24 hours, along with their morphological profiles depicted as heatmaps. All data points represent per-well means of 3-4 independent differentiations. Dashed lines show mean Z-scores for positive (“A”) and negative (“B”) controls. Shaded areas represent ±1SD. FIG. 5G shows the chemical synthesis and structure of compound C-01. FIGS. 5H-5I show dose-response curves for ATG9A and DAGLB ratios in iPSC-neurons from a patient with SPG50 (FIG. 5H) and an additional patient with SPG47 (FIG. 5I) after prolonged treatment with C-01 for 72 hours, along with the morphologic profile depicting changes in cellular ATG9A and DAGLB distribution, TGN intensity and morphology and cell count. All data points represent per-well means of 2 independent differentiations. Dashed lines show mean Z-scores for positive (“A”) and negative (“B”) controls. Shaded areas represent ±1 SD.

FIGS. 6A-6F show target deconvolution using bulk RNA sequencing and weighted gene co-expression network analysis in AP4B1^{KO} SH-SY5Y cells treated with C-01. FIG. 6A shows hierarchical clustering of 12 samples using average linkage showed two main clusters based on treatment with vehicle vs. C-01, irrespective of cell line. FIG. 6B shows a cluster dendrogram of 18,506 expressed genes based on topological overlap. Clusters of co-expressed genes (“modules”) were isolated using hierarchical clustering and adaptive branch pruning. FIG. 6C shows a heatmap visualization of the correlation of gene expression profiles (“module eigengene”, ME) of each module with measured traits. Pearson correlation coefficients are shown for each cell of the heatmap. FIG. 6D shows intramodular analysis of module membership (MM) and gene significance (GS) for highly correlated modules, allowing identification of genes that have high significance with treatment as well as high connectivity to their modules. FIG. 6E shows ME expression profiles for the top 5 co-expressed modules. FIG. 6F shows that gene ontology enrichment analysis showed enriched pathways in 3/5 modules. Pathways were considered differentially expressed with an FDR<0.05.

FIGS. 7A-7D show target deconvolution using unbiased quantitative proteomics in AP4B1^{KO} SH-SY5Y cells and AP-4-HSP patient-derived iPSC-neurons treated with C-01. FIGS. 7A-7C show differential protein enrichment analysis. Statistical testing was done using protein-wise linear models and empirical Bayes statistics. Proteins were considered as differentially enriched with a false discovery rate of <0.05 and a log 2 fold change >0.3. FIG. 7A shows SH-SY5Y cells: 8141 unique proteins were analyzed. PCA of the top 500 variable proteins shows robust separation between experimental conditions. The volcano plot summarizes differential protein enrichment for AP4B1^{WT} and AP4B1^{KO} cells pooled into two groups, vehicle vs. C-01 treated. Differentially enriched proteins are depicted in black. Proteins with the most consistent enrichment profiles across all experimental conditions (see FIGS. 14A-14D) are labeled and have an adjacent arrow. FIG. 7B shows iPSC-derived neurons: 7386 unique proteins were analyzed. PCA of the top 500 variable proteins shows robust separation between experimental conditions. The volcano plot summarize differential protein enrichment for control and patient-derived neurons pooled into two groups, vehicle vs. C-01 treated. Differentially enriched proteins are depicted in black. Proteins with the most consistent enrichment profiles across all experimental conditions (see FIGS. 14E-14H) are labeled and have an adjacent arrow. FIG. 7C shows the integrated analysis of SH-SY5Y cells and iPSC-derived neurons: 5357 unique proteins were analyzed. The volcano plot summarizes differential protein enrichment for control and AP-4-deficient cells pooled into two groups, vehicle vs. C-01. Proteins with the most consistent enrichment profiles across all experimental conditions (see FIGS. 14I-14L) are labeled and have an adjacent arrow. The dot plot summarizes dysregulated Reactome pathways of the pooled analysis. Pathways were considered differentially expressed with an FDR<0.05. FIG. 7D shows the RAB protein family members RAB1B, RAB3C and RAB12 showed the most consistent profiles in response to C-01 treatment and were selected for further analysis. LFQ intensities in SH-SY5Y cells (AP4B1^{WT} and AP4B1^{KO} pooled) and neurons (control and patient pooled) are shown. Statistical testing was done using pairwise T-tests. P-values were adjusted for multiple testing using the Benjamini-Hochberg procedure.

FIGS. 8A-8I show that RAB3C and RAB12 are involved in C-01-mediated vesicle trafficking and enhancement of autophagic flux. FIG. 8A shows that LFQ intensities of RAB3C and RAB12 in AP4B1^{WT} (n=11 samples) and AP4B1^{KO} (n=10 samples) SH-SY5Y cells, as well as control (n=6 samples) and patient-derived (n=6 samples) iPSC-derived neurons show a high degree of correlation measured by the Pearson correlation coefficient (r). While there was no difference between genotypes (not shown), C-01 treated cells showed reduced protein levels of both RAB3C and RAB12. FIG. 8B shows AP4B1^{KO} SH-SY5Y cells were transfected for 72 hours with RNPs targeting RAB3C, RAB12 or both compared to NLRP5 as a non-essential control. Vehicle vs. C-01 treatment at a concentration of 5 μM was administered for 24 hours. Each experimental condition was tested in 8-12 wells from 2-3 independent plates. The dashed line represents a reduction of the ATG9A ratio of −2 SD compared to the negative control (AP4B1^{KO}+sgNLRP5). Knockout of RAB12 did not significantly alter the ATG9A ratio, while RAB3C knockout led to a reduction of −2 SD. Combining the knockout of RAB3C and RAB12 did not result in an additive effect. However, both RAB3C and RAB12 knockout potentiated the effect of C-01 treatment on ATG9A translocation, which was further enhanced by combined knockout. FIGS. 8C-8F show representative western blots of whole cell lysates. Cells were treated with vehicle vs. C-01 at a concentration of 5 μM for 72 hours. All experiments were performed in four biological replicates. AP4E1 levels were reduced in AP4B1^{KO} cells, indicating reduced AP-4 complex formation. ATG9A ratios were significantly increased in AP4B1^{KO} cells and were not altered by C-01 treatment. By contrast, the conversion of LC3-I to LC3-II was significantly elevated in response to C-01 in both AP4B1^{WT} and AP4B1^{KO} cells. To confirm that this increase was due to an increase in autophagic flux, autophagosome-lysosome fusion was blocked by adding bafilomycin A1 at a concentration of 100 nM for 4 hours prior to cell harvest. FIGS. 8G-8I show western blots of whole cell lysates of AP4B1^{KO} SH-SY5Y cells transfected for 72 hours with RNPs against RAB3C, RAB12 or both, compared to NLRP5. Vehicle vs. C-01 treatment was administered for 48 hours. While neither RAB3C (FIG. 8G) nor RAB12 (FIG. 8H) knockout alone led to an increase in baseline LC3-II, the combined knockout raised the LC3-II to LC3-I ratio to levels achieved with C-01 treatment alone (FIG. 8I). In response to bafilomycin A1 treatment (100 nM for 4 hours) both RAB3C knockout alone and the combined knockout of RAB3C and RAB12 led to a significant increase in LC3-II to LC3-I ratios. Statistical testing in all experiments was done using pairwise T-tests. P-values were adjusted for multiple testing using the Benjamini-Hochberg procedure.

FIGS. 9A-9B show quality metrics of the ATG9A translocation assay in the primary screen and counter-screen. Assay performance was monitored in the (FIG. 9A) primary screen and (FIG. 9B) counter-screen using criteria proposed by Zhang et al. and included a Z′ robust ≥0.3, a strictly standardized median difference (SSMD)≥3 and an inter-assay coefficient of variation ≤10%. All metrics were calculated with respect to the positive and negative controls of the same assay plate to avoid bias by inter-plate variability. Predefined thresholds (“A” lines) were met by all assay plates.

FIGS. 10A-10B show a summary of the counter-screen in AP-4-HSP patient-derived fibroblasts. Overview of the counter-screen of the 503 active compounds identified in the primary screen. To assess for dose-dependent effects, compounds were screened in AP-4-HSP patient-derived fibroblasts in 384-well microplates using 11-point titrations ranging from 40 nM to 40 μM. All concentrations were screened in duplicates. Active compounds were a priori defined as those reducing the ATG9A ratio by at least 3 SD compared to negative controls, in more than one concentration. Toxicity was defined as a reduction of cell count of at least 2 SD compared to the negative control. “A” dotted lines represent the mean of the positive controls, while “B” dotted lines indicate the mean of the negative controls. Triangles indicate toxic concentrations. ED50 are indicated where possible. 17 compounds demonstrated a clear and reproducible dose-response relationship and raised no suspicion for autofluorescence on automated and manual review. 34 compounds were active but showed autofluorescence or resulted in imaging artifacts. Dose-response curves for all 503 compounds tested in the secondary screen.

FIG. 11 shows that the orthogonal screen identified 11 compounds that showed no activity in AP4B1^{KO} SH-SY5Y cells. Eleven of 16 compounds were excluded due to either lacking activity (D-01, E-01, L-01, M-01, N-01, 0-01, P-01), suspicion for artefacts or autofluorescence (I-01, J-01, K-01), or obvious changes in cellular morphology (A-01).

FIGS. 12A-12F show multiparametric profiling of 5 active compounds in AP4B1^{KO} SH-SY5Y cells. PCA analysis of 85 extracted features of the nucleus, cytoskeleton/global cell morphology, TGN and ATG9A vesicles is shown. FIG. 12A shows baseline analysis of AP4B1^{WT} and AP4B1^{KO} cells. Cell lines clustered closely together and were only separated by the ATG9A signal. FIGS. 12B-12F show spatial clustering of the 5 active compounds in relation to the positive and negative controls. Compound concentrations are depicted by the legend. While C-01 treated AP4B1^{KO} cells clustered closely with the controls (FIG. 12C), suggesting no significant off-target effects, all other compounds led to changes in overall cellular morphology in a dose-dependent manner. The most significant changes were seen for F-01 (FIG. 12D) and H-01 (FIG. 12F), suggesting off-target effects.

FIGS. 13A-13C show that bulk RNA sequencing in AP4B1^{KO} SH-SY5Y cells treated with C-01 shows a small number of differentially expressed genes, mainly involved in ER stress response. FIG. 13A shows volcano plots depicting the results of bulk RNA Sequencing in different experimental conditions in SH-SY5Y cells (AP4B1^{WT} vs. AP4B1^{KO} treated with vehicle, AP4B1^{WT} treated with vehicle vs. AP4B1^{WT} treated with C-01, AP4B1^{KO} treated with vehicle vs. AP4B1^{KO} treated with C-01, AP4B1^{WT} and AP4B1^{KO} cells pooled in two groups, vehicle vs. C-01). Differential expression analysis was done following the TREAT approach developed by McCarthy and Smyth (2009). Dots labeled with “A” represent differentially expressed genes with a log 2 fold change >0.3 and an FDR<0.05. FIG. 13B shows that gene ontology analysis shows enriched pathways of the pooled analysis. Pathways were considered differentially expressed with an FDR<0.05. FIG. 13C portrays a Gene-Concept Network showing differentially expressed genes and their pathway membership.

FIGS. 14A-14L show unbiased quantitative proteomics in AP4B1^{KO} SH-SY5Y cells and AP-4-HSP patient-derived iPSC-neurons treated with C-01. FIGS. 14A-14D show SH-SY5Y cells: 8141 unique proteins were analyzed. Volcano plots summarize differential protein enrichment for different experimental conditions: FIG. 14A shows AP4B1^{WT} vs. AP4B1^{KO} treated with vehicle, FIG. 14B shows AP4B1^{WT} treated with vehicle vs. AP4B1^{WT} treated with C-01, FIG. 14C shows AP4B1^{KO} treated with vehicle vs. AP4B1^{KO} treated with C-01, and FIG. 14D shows AP4B1^{WT} and AP4B1^{KO} cells pooled treated with vehicle vs. C-01. Differentially enriched proteins are depicted in black. FIGS. 14E-14H show iPSC-derived neurons: 7386 unique proteins were analyzed. Volcano plots summarize differential protein enrichment for different experimental conditions. FIG. 14E shows controls vs. patient-derived neurons treated with vehicle, FIG. 14F shows controls treated with vehicle vs. controls treated with C-01, FIG. 14G shows patient-derived neurons treated with vehicle vs. patient-derived neurons treated with C-01, and FIG. 14H shows controls and patient-derived neurons pooled in two groups, treated with vehicle vs. C-01. Differentially enriched proteins are depicted in black. FIGS. 14I-14L show integrated analysis of SH-SY5Y cells and iPSC-derived neurons: 5357 unique proteins were analyzed. Volcano plots summarize differential protein enrichment for different experimental conditions. FIG. 14I shows controls vs. AP-4-deficient cells treated with vehicle, FIG. 14J shows controls treated with vehicle vs. controls treated with C-01, FIG. 14K shows AP-4-deficient cells treated with vehicle vs. AP-4-deficient cells treated with C-01, FIG. 14L shows controls and AP-4-deficient cells pooled into two groups, vehicle vs. C-01. Differentially enriched proteins are depicted in black. In all datasets statistical testing for differential protein enrichment was done using protein-wise linear models and empirical Bayes statistics. Proteins were considered as differentially enriched with a false discovery rate of <0.05 and a log 2 fold change >0.3.

FIGS. 15A-15E show mRNA transcript expression and correlation analysis of RAB3C and RAB12. Normalized mRNA transcript counts for RAB3C (FIG. 15A, FIG. 15C) and RAB12 (FIG. 15B, FIG. 15D) across different experimental conditions in SH-SY5Y cells (AP4B1^{WT} treated with vehicle, AP4B1^{WT} treated with C-01, AP4B1^{KO} treated with vehicle, AP4B1^{KO} treated with C-01) (FIG. 15A, FIG. 15B), as well as AP4B1^{WT} and AP4B1^{KO} cells pooled treated with vehicle vs. C-01 (FIG. 15C, FIG. 15D). No significant differences were detected. Statistical testing was done using pairwise T-tests. P-values have been adjusted for multiple testing using the Benjamini-Hochberg procedure. FIG. 15E shows correlation analysis of RAB3C and RAB12 gene expression in AP4B1^{WT} (n=6 samples) and AP4B1^{KO} (n=6 samples) SH-SY5Y cells shows a moderate inverse correlation measured by the Pearson correlation coefficient (r).

FIGS. 16A-16D show original western blots. FIG. 16A shows uncropped, original blots corresponding to FIG. 8C. FIG. 16B shows an uncropped, original blot corresponding to FIG. 8G. FIG. 16C shows an uncropped, original blot corresponding to FIG. 8H. FIG. 16D shows an uncropped, original blot corresponding to FIG. 8I.

FIGS. 17A-B show a dose response curve and cell count number for exemplary Compound 10 in an assay testing for its effectiveness in the treatment of AP-4 deficiency. Corresponds to AP4B1-KO+Compound 10 in Table 1.

FIGS. 18A-B show a dose response curve and cell count number for exemplary Compound 17 in an assay testing for its effectiveness in the treatment of AP-4 deficiency. Corresponds to AP4B1-KO+Compound 17 in Table 1.

### DETAILED DESCRIPTION OF CERTAIN EMBODIMENTS

The present disclosure describes the use of intracellular ATG9A mislocalization as a cellular readout for AP-4 deficiency to develop a large-scale, automated, multi-parametric, unbiased phenotypic small molecule screen for modulators of ATG9A trafficking in patient-derived cellular models. A diverse library of novel small molecules were screened in AP-4-deficient patient fibroblasts to identify compounds that redistribute ATG9A from the TGN to the cytoplasm. Through a series of orthogonal assays in neuronal cells, including differentiated AP4B1KO SH-SY5Y cells and iPSC-derived neurons from patients, compounds that restore neuronal phenotypes of AP-4-deficiency were discovered.

Accordingly, described herein are compounds of Formula (I). The compounds restore neuronal phenotypes of AP-4-deficiency, modulate intracellular vesicle trafficking, and increase autophagic flux.

### Compounds

In one aspect, the present disclosure provides compounds of Formula (I):

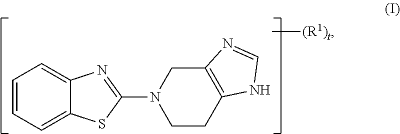

and pharmaceutically acceptable salts, solvates, hydrates, polymorphs, co-crystals, tautomers, stereoisomers, isotopically labeled derivatives, and prodrugs thereof, wherein:
each occurrence of R¹ is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —C(═O)NR^{A}S(O)₂R^{A}, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, OS(O)₂R^{A}, —S(O)₂NR^{A}C(O)R^{A}, —S(O)₂N(R^{A})₂, —S(O)₂OR^{A} or —S(O)₂R^{A}; or two R¹ groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0 or a positive integer; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
wherein, when present, each occurrence of R¹ is bound to any substitutable atom of the compound.

In certain embodiments, the compound is not of the formula of any one or more of the following:

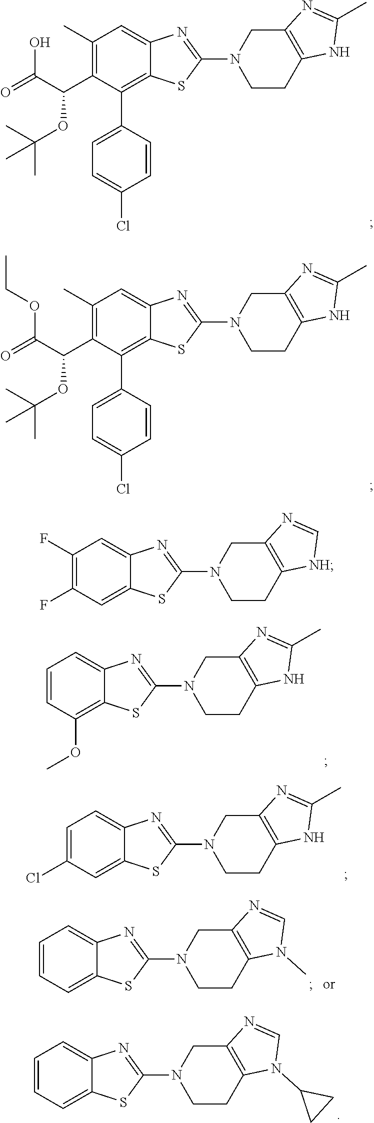

### R¹

As described herein, each occurrence of R¹ is, independently, hydrogen, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —C(═O)NR^{A}S(O)₂R^{A}, —NO₂, —NR^{A}C(═O)R^{A}, —NR^{A}C(═O)OR^{A}, NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, —OS(O)₂R^{A}, —S(O)₂NR^{A}C(O)R^{A}, —S(O)₂N(R^{A})₂, —S(O)₂OR^{A} or S(O)₂R^{A}; or two R¹ groups are joined to form a substituted or unsubstituted carbocyclyl ring, a substituted or unsubstituted aryl ring, a substituted or unsubstituted heterocyclyl ring, or a substituted or unsubstituted heteroaryl ring.

In certain embodiments, each occurrence of R¹ is, independently, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —C(═O)NR^{A}S(O)₂R^{A}, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, OS(O)₂R^{A}, —S(O)₂NR^{A}C(O)R^{A}, —S(O)₂N(R^{A})₂, —S(O)₂OR^{A} or —S(O)₂R^{A}; or two R¹ groups are joined to form a substituted or unsubstituted carbocyclyl ring, a substituted or unsubstituted aryl ring, a substituted or unsubstituted heterocyclyl ring, or a substituted or unsubstituted heteroaryl ring.

In certain embodiments, each occurrence of R¹ is, independently, halogen, substituted or unsubstituted acyl, substituted or unsubstituted alkyl, substituted or unsubstituted alkenyl, substituted or unsubstituted alkynyl, substituted or unsubstituted heteroaliphatic, —OR^{A}, —N(R^{A})₂, —SR^{A}, —CN, —SCN, —C(═O)R^{A}, —C(═O)OR^{A}, —C(═O)N(R^{A})₂, —C(═O)NR^{A}S(O)₂R^{A}, —S(O)₂NR^{A}C(O)R^{A}, —S(O)₂N(R^{A})₂, —S(O)₂OR^{A} or —S(O)₂R^{A}.

In certain embodiments, each occurrence of R¹ is, independently, substituted or unsubstituted acyl, substituted or unsubstituted alkyl, substituted or unsubstituted heteroalkyl, —OR^{A}, —C(═O)OR^{A}, or —C(═O)N(R^{A})₂.

In certain embodiments, each occurrence of R¹ is, independently, substituted or unsubstituted alkyl, —OR^{A}, —C(═O)OR^{A}, or —C(═O)N(R^{A})₂.

In certain embodiments, each occurrence of R¹ is, independently, substituted or unsubstituted alkyl, —OR^{A}, —C(═O)OR^{A}, or —C(═O)N(R^{A})₂; wherein each occurrence of R^{A} is, independently, hydrogen, substituted or unsubstituted alkyl, or substituted or unsubstituted heteroalkyl.

In certain embodiments, each occurrence of R¹ is, independently, unsubstituted alkyl, —OR^{A}, —C(═O)OR^{A}, or —C(═O)N(R^{A})₂; wherein each occurrence of R^{A} is, independently, hydrogen, substituted or unsubstituted alkyl, or substituted or unsubstituted heteroalkyl.

In certain embodiments, each occurrence of R¹ is, independently, unsubstituted alkyl, —OR^{A}, —C(═O)OR^{A}, or —C(═O)N(R^{A})₂; wherein each occurrence of R^{A} is, independently, hydrogen, substituted or unsubstituted C₁₋₄alkyl, or substituted or unsubstituted hetero C₁₋₄alkyl.

In certain embodiments, each occurrence of R¹ is, independently, unsubstituted alkyl, —OR^{A}, —C(═O)OR^{A}, or —C(═O)N(R^{A})₂; wherein each occurrence of R^{A} is, independently, hydrogen, unsubstituted C₁₋₄alkyl, or unsubstituted hetero C₁₋₄alkyl.

In certain embodiments, each occurrence of R¹ is, independently, unsubstituted C₁₋₄alkyl, —OH, —OC₁₋₄alkyl, —C(═O)OC₁₋₄ alkyl, or —C(═O)NH—(C₁₋₄alkylene)-OC₁₋₄ alkyl.

In certain embodiments, each occurrence of R¹ is, independently, hydrogen, —CH₃, —OH, —OCH₃, —C(═O)OCH₃, or —C(═O)NH(CH₂CH₂)OCH₃. In certain embodiments, each occurrence of R¹ is, independently, —CH₃, —OH, —OCH₃, —C(═O)OCH₃, or —C(═O)NH(CH₂CH₂)OCH₃.

In certain embodiments, each occurrence of R¹ is, independently, —C(═O)OR^{A}. In certain embodiments, each occurrence of R¹ is, independently, —C(═O)OR^{A}; wherein each occurrence of R^{A} is, independently, substituted or unsubstituted alkyl. In certain embodiments, each occurrence of R¹ is, independently, —C(═O)OR^{A}; wherein each occurrence of R^{A} is, independently, substituted or unsubstituted C₁₋₄alkyl. In certain embodiments, each occurrence of R¹ is, independently, —C(═O)OR^{A}; wherein each occurrence of R^{A} is, independently, unsubstituted C₁₋₄alkyl. In certain embodiments, each occurrence of R¹ is, independently, —C(═O)OR^{A}; wherein each occurrence of R^{A} is, independently, unsubstituted C₁₋₃alkyl. In certain embodiments, each occurrence of R¹ is, independently, —C(═O)OR^{A}; wherein each occurrence of R^{A} is, independently, unsubstituted C₁₋₂alkyl. In certain embodiments, each occurrence of R¹ is, independently, —C(═O)OCH₃.

In certain embodiments, each occurrence of R¹ is, independently, —C(═O)N(R^{A})₂; wherein each occurrence of R^{A} is, independently, hydrogen, or substituted or unsubstituted heteroalkyl. In certain embodiments, each occurrence of R¹ is, independently, —C(═O)N(R^{A})₂; wherein each occurrence of R^{A} is, independently, hydrogen, or substituted or unsubstituted hetero C₁₋₄alkyl. In certain embodiments, each occurrence of R¹ is, independently, —C(═O)N(R^{A})₂; wherein each occurrence of R^{A} is, independently, hydrogen, or unsubstituted hetero C₁₋₄alkyl. In certain embodiments, each occurrence of R¹ is, independently, —C(═O)NH—(C₁₋₄alkylene)-OC₁₋₄ alkyl. In certain embodiments, each occurrence of R¹ is, independently, —C(═O)NH—(C₁₋₃alkylene)-OC₁₋₃alkyl. In certain embodiments, each occurrence of R¹ is, independently, —C(═O)NH—(C₁₋₂alkylene)-OC₁₋₂alkyl. In certain embodiments, each occurrence of R¹ is, independently, —C(═O)NH(CH₂CH₂)OCH₃.

In certain embodiments, each occurrence of R¹ is, independently, substituted or unsubstituted alkyl. In certain embodiments, each occurrence of R¹ is, independently, substituted or unsubstituted C₁₋₄alkyl. In certain embodiments, each occurrence of R¹ is, independently, unsubstituted C₁₋₄alkyl. In certain embodiments, each occurrence of R¹ is, independently, unsubstituted C₁₋₃alkyl. In certain embodiments, each occurrence of R¹ is, independently, unsubstituted C₁₋₂alkyl. In certain embodiments, each occurrence of R¹ is, independently, —CH₃.

In certain embodiments, each occurrence of R¹ is, independently, —OR^{A}. In certain embodiments, each occurrence of R¹ is, independently, —OR^{A}, wherein each occurrence of R^{A} is, independently, hydrogen or substituted or unsubstituted alkyl. In certain embodiments, each occurrence of R¹ is, independently, —OR^{A}, wherein each occurrence of R^{A} is, independently, hydrogen or unsubstituted C₁₋₄alkyl. In certain embodiments, each occurrence of R¹ is, independently, —OR^{A}, wherein each occurrence of R^{A} is, independently, hydrogen or unsubstituted C₁₋₄alkyl. In certain embodiments, each occurrence of R¹ is, independently, —OR^{A}, wherein each occurrence of R^{A} is, independently, hydrogen or unsubstituted C₁₋₃alkyl. In certain embodiments, each occurrence of R¹ is, independently, —OR^{A}, wherein each occurrence of R^{A} is, independently, hydrogen or unsubstituted C₁₋₂alkyl. In certain embodiments, each occurrence of R¹ is, independently, —OH or —OCH₃. In certain embodiments, each occurrence of R¹ is, independently, —OH. In certain embodiments, each occurrence of R¹ is, independently, —OCH₃.

In certain embodiments, each occurrence of R¹ is, independently, hydrogen, —OH or —CH₃. In certain embodiments, each occurrence of R¹ is, independently, hydrogen. In certain embodiments, each occurrence of R¹ is, independently, —OH or —CH₃.

As described herein, t is 0 or a positive integer. In certain embodiments, t is an integer from 0-10. In certain embodiments, t is an integer from 0-8. In certain embodiments, t is an integer from 0-6. In certain embodiments, t is an integer from 0-5. In certain embodiments, t is an integer from 0-4. In certain embodiments, t is an integer from 0-3. In certain embodiments, t is an integer from 0-2. In certain embodiments, t is 0 or 1. In certain embodiments, t is 1. In certain embodiments, t is 0.

### Embodiments of Formula (I)

In certain embodiments, the compound of Formula (I) is a compound of Formula (I-a):

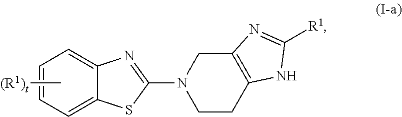

or a pharmaceutically acceptable salt, co-crystal, tautomer, stereoisomer, solvate, hydrate, polymorph, isotopically enriched derivative, or prodrug thereof, wherein R¹ and t are as defined herein.

In certain embodiments, the compound of Formula (I) is a compound of Formula (I-b):

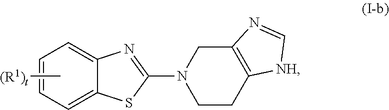

or a pharmaceutically acceptable salt, co-crystal, tautomer, stereoisomer, solvate, hydrate, polymorph, isotopically enriched derivative, or prodrug thereof, wherein R¹ and t are as defined herein.

In certain embodiments, the compound of Formula (I) is a compound of Formula (I-c):

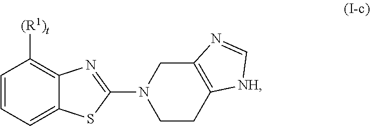

or a pharmaceutically acceptable salt, co-crystal, tautomer, stereoisomer, solvate, hydrate, polymorph, isotopically enriched derivative, or prodrug thereof, wherein R¹ and t are as defined herein.

In certain embodiments, the compound of Formula (I) is a compound of Formula (I-d):

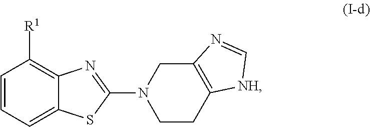

or a pharmaceutically acceptable salt, co-crystal, tautomer, stereoisomer, solvate, hydrate, polymorph, isotopically enriched derivative, or prodrug thereof, wherein R¹ is as defined herein.

In certain embodiments, the compound of Formula (I) is a compound of the formula:

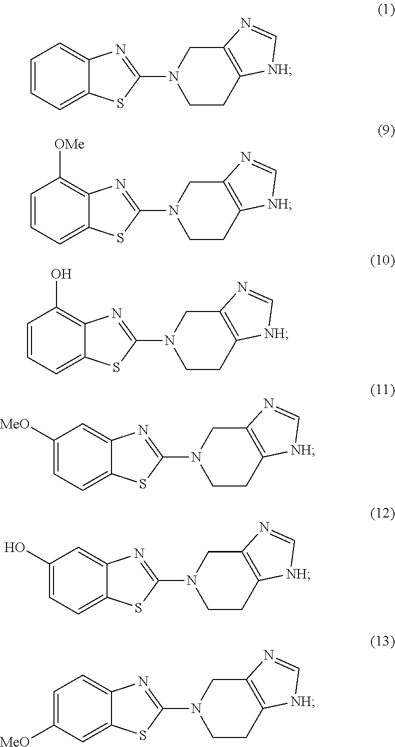

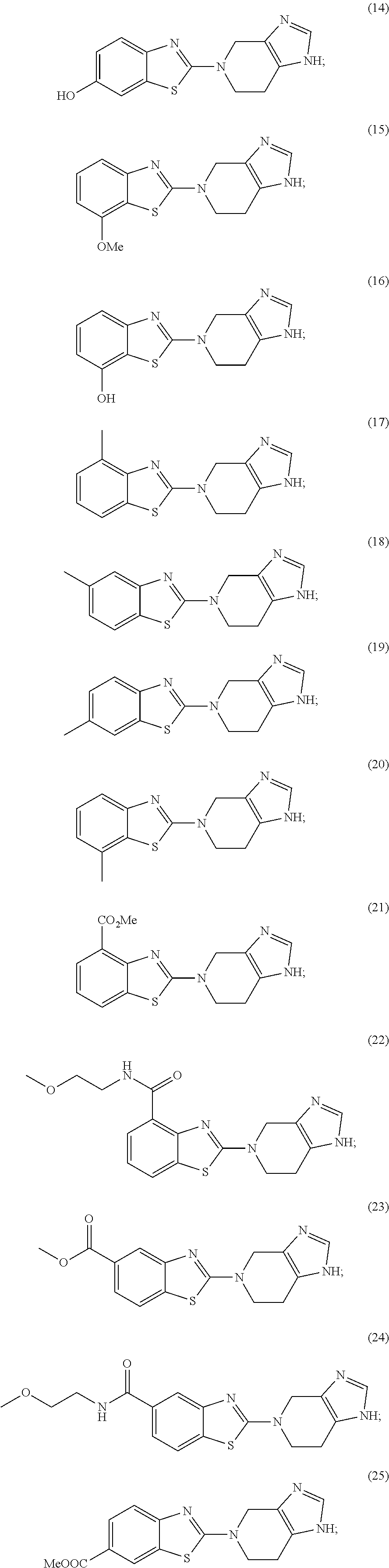

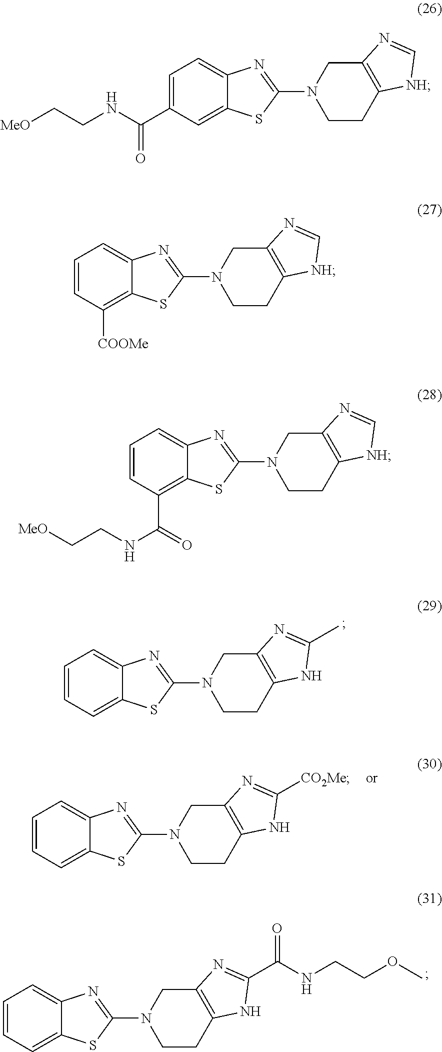 [SMILES: COCCNC(=O)c1ccc2nc(N3CCc4[nH]cnc4C3)sc2c1 ; COC(=O)c1cccc2nc(N3CCc4[nH]cnc4C3)sc12 ; COCCNC(=O)c1cccc2nc(N3CCc4[nH]cnc4C3)sc12 ; Cc1nc2c([nH]1)CCN(c1nc3ccccc3s1)C2 ; COC(=O)c1nc2c([nH]1)CCN(c1nc3ccccc3s1)C2 ; COCCNC(=O)c1nc2c([nH]1)CCN(c1nc3ccccc3s1)C2]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

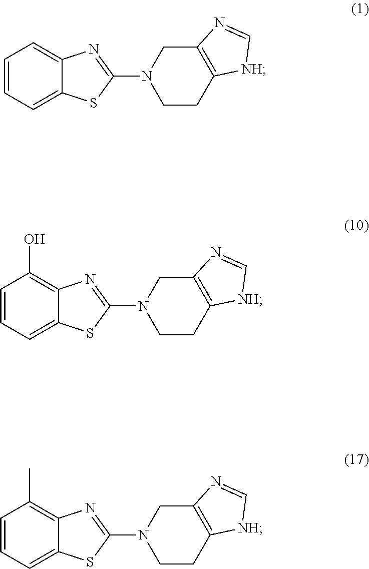 [SMILES: c1ccc2sc(N3CCc4[nH]cnc4C3)nc2c1 ; Oc1cccc2sc(N3CCc4[nH]cnc4C3)nc12 ; Cc1cccc2sc(N3CCc4[nH]cnc4C3)nc12]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

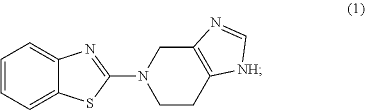 [SMILES: c1ccc2sc(N3CCc4[nH]cnc4C3)nc2c1]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

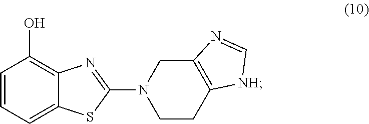 [SMILES: Oc1cccc2sc(N3CCc4[nH]cnc4C3)nc12]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound of the formula:

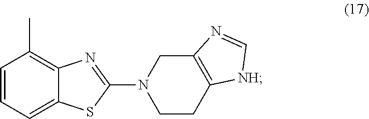 [SMILES: Cc1cccc2sc(N3CCc4[nH]cnc4C3)nc12]

or a pharmaceutically acceptable salt, co-crystal, tautomer, stereoisomer, solvate, hydrate, polymorph, isotopically enriched derivative, or prodrug thereof.

### Pharmaceutical Compositions, Kits, and Administration

The present disclosure provides pharmaceutical compositions comprising a compound of the disclosure (e.g., a compound of Formula (I)), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of the disclosure (e.g., a compound of Formula (I)), or a pharmaceutically acceptable salt thereof, and a pharmaceutically acceptable excipient.

In certain embodiments, a compound of the disclosure (e.g., a compound of Formula (I)) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a neurological disease or disorder in a subject in need thereof. In certain embodiments, the effective amount is an amount effective for preventing a neurological disease or disorder in a subject in need thereof. In certain embodiments, the effective amount is an amount effective for treating a neurological disease or disorder associated with aberrant protein trafficking. In certain embodiments, the effective amount is an amount effective for treating a neurological disease or disorder associated with aberrant protein trafficking in adaptor protein complex 4 (AP-4)-deficiency. In certain embodiments, the effective amount is an amount effective for treating a hereditary spastic paraplegia (HSP). In certain embodiments, the effective amount is an amount effective for treating Adaptor protein complex 4 (AP-4)-related hereditary spastic paraplegia (AP-4-HSP) (e.g., AP4B1-associated SPG47 (OMIM #614066), AP4M1-associated SPG50 (OMIM #612936), AP4E1-associated SPG51 (OMIM #613744), AP4S1-associated SPG52 (OMIM #614067)).

In certain embodiments, the effective amount is an amount effective for modulating Autophagy Related 9A (ATG9A) trafficking in or from a cell. In certain embodiments, the effective amount is an amount effective for modulating intracellular vesicle trafficking and increasing autophagic flux in a cell.

Pharmaceutical compositions described herein can be prepared by any method known in the art of pharmacology. In general, such preparatory methods include the steps of bringing the composition comprising a compound of the disclosure (e.g., a compound of Formula (I)) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

The compound and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both. The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a neurological disease or disorder (e.g., hereditary spastic paraplegia (HSP)) in a subject in need thereof. In certain embodiments, the kits are useful for preventing a neurological disease or disorder (e.g., hereditary spastic paraplegia (HSP)) in a subject in need thereof. In certain embodiments, the kits are useful for reducing the risk of developing a neurological disease or disorder (e.g., hereditary spastic paraplegia (HSP)) in a subject in need thereof. In certain embodiments, the kits are useful for modulating Autophagy Related 9A (ATG9A) trafficking in or from a cell. In certain embodiments, the kits are useful for modulating intracellular vesicle trafficking and increasing autophagic flux in a subject and/or a cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition.

### Methods of Treatment

The present disclosure provides methods for treating cancer. In certain embodiments, the present disclosure provides a method for treating a neurological disease or disorder. In certain embodiments, the present disclosure provides a method of treating a neurological disease or disorder associated with aberrant protein trafficking. In certain embodiments, the present disclosure provides a method of treating a neurological disease or disorder associated with aberrant protein trafficking in adaptor protein complex 4 (AP-4)-deficiency. In certain embodiments, the present disclosure provides a method of treating a hereditary spastic paraplegia (HSP). In certain embodiments, the present disclosure provides a method of treating Adaptor protein complex 4 (AP-4)-related hereditary spastic paraplegia. In certain embodiments, the present disclosure provides a method of treating AP4B1-associated SPG47 (OMIM #614066). In certain embodiments, the present disclosure provides a method of treating AP4M1-associated SPG50 (OMIM #612936). In certain embodiments, the present disclosure provides a method of treating AP4E1-associated SPG51 (OMIM #613744). In certain embodiments, the present disclosure provides a method of treating AP4S1-associated SPG52 (OMIM #614067).

In certain embodiments, the present disclosure provides a method of modulating Autophagy Related 9A (ATG9A) trafficking in or from a cell. In certain embodiments, the present disclosure provides a method of modulating intracellular vesicle trafficking and increasing autophagic flux in a subject and/or a cell. In certain embodiments, the cell is a mammalian cell. In certain embodiments, the cell is a human cell. In certain embodiments, the cell is in a subject. In certain embodiments, the cell is in a mammal. In certain embodiments, the cell is in a human.

In certain embodiments, the methods of the disclosure comprise administering to a subject an effective amount of a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as a compound of the disclosure (e.g., a compound of Formula (I)), or at different times than a compound of the disclosure (e.g., a compound of Formula (I)). For example, a compound of the disclosure (e.g., a compound of Formula (I)) and any additional pharmaceutical agent(s) may be on the same dosing schedule or different dosing schedules. All or some doses of a compound of the disclosure (e.g., a compound of Formula (I)) may be administered before all or some doses of an additional pharmaceutical agent, after all or some does an additional pharmaceutical agent, within a dosing schedule of an additional pharmaceutical agent, or a combination thereof.

The timing of administration of a compound of the disclosure (e.g., a compound of Formula (I)) and additional pharmaceutical agents may be different for different additional pharmaceutical agents.

In certain embodiments, the additional pharmaceutical agent comprises an agent useful in the treatment of a neurological disease or disorder. In certain embodiments, the additional pharmaceutical agent is useful in the treatment of a neurological disease or disorder associated with aberrant protein trafficking. In certain embodiments, the additional pharmaceutical agent is useful in the treatment of a neurological disease or disorder associated with aberrant protein trafficking in adaptor protein complex 4 (AP-4)-deficiency. In certain embodiments, the additional pharmaceutical agent is useful in the treatment of a hereditary spastic paraplegia (HSP). In certain embodiments, the additional pharmaceutical agent is useful in the treatment of Adaptor protein complex 4 (AP-4)-related hereditary spastic paraplegia (AP-4-HSP) (e.g., AP4B1-associated SPG47 (OMIM #614066), AP4M1-associated SPG50 (OMIM #612936), AP4E1-associated SPG51 (OMIM #613744) and AP4S1-associated SPG52 (OMIM #614067)).

In another aspect, the present disclosure provides methods for modulating Autophagy Related 9A (ATG9A) trafficking in or from a cell, the method comprising contacting the cell with a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In certain embodiments, the cell is in a subject. In certain embodiments, the contacting is in a biological sample. In certain embodiments, the contacting results in an increase in trafficking of ATG9A out of the trans-Golgi network (TGN). In certain embodiments, the contacting results in a decrease of ATG9A in the trans-Golgi network (TGN). In certain embodiments, the contacting results in a decrease of ATG9A in the trans-Golgi network (TGN) by at least 5%, at least 10%, at least 15%, at least 20%, at least 25%, at least 30%, at least 35%, at least 40%, at least 45%, at least 50%, at least 60%, at least 70%, at least 80%, at least 90%, or at least 95%. In certain embodiments, the contacting results in a decrease of the ratio of the concentration of ATG9A in the trans-Golgi network (TGN) to the concentration of ATG9A in the cytoplasm. In certain embodiments, the ratio of the concentration of ATG9A in the trans-Golgi network (TGN) to the concentration of ATG9A in the cytoplasm is less than or equal to 1:1, less than or equal to 1.1:1, less than or equal to 1.2:1, less than or equal to 1.3:1, less than or equal to 1.4:1, less than or equal to 1.5:1, less than or equal to 1.6:1, less than or equal to 1.7:1, less than or equal to 1.8:1, less than or equal to 1.9:1, or less than or equal to 2:1 after contacting the cell with a compound of the disclosure. In certain embodiments, the ratio of the concentration of ATG9A in the trans-Golgi network (TGN) to the concentration of ATG9A in the cytoplasm is at least 1:1, at least 1.1:1, at least 1.2:1, at least 1.3:1, at least 1.4:1, at least 1.5:1, at least 1.6:1, at least 1.7:1, at least 1.8:1, at least 1.9:1, or at least 2:1 after contacting the cell with a compound of the disclosure. In certain embodiments, the contacting is in vitro. In certain embodiments, the contacting is in vivo. In certain embodiments, the cell is a mammalian cell. In certain embodiments, the cell is a human cell.

In another aspect, the present disclosure provides methods for modulating intracellular vesicle trafficking and increasing autophagic flux in a cell, the method comprising contacting the cell with a compound of the disclosure (e.g., a compound of Formula (I)), or a pharmaceutically acceptable salt, co-crystal, tautomer, stereoisomer, solvate, hydrate, polymorph, isotopically enriched derivative, or prodrug, or composition thereof. In certain embodiments, the cell is in a subject. In certain embodiments, the cell is in a biological sample. In certain embodiments, the contacting is in vitro. In certain embodiments, the contacting is in vivo. In certain embodiments, the cell is a mammalian cell.

In certain embodiments, the cell is a human cell.

### Examples

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

Primary Screening of 28,864 Compounds in Fibroblasts from AP-4-HSP Patients Identified 503 Active Compounds

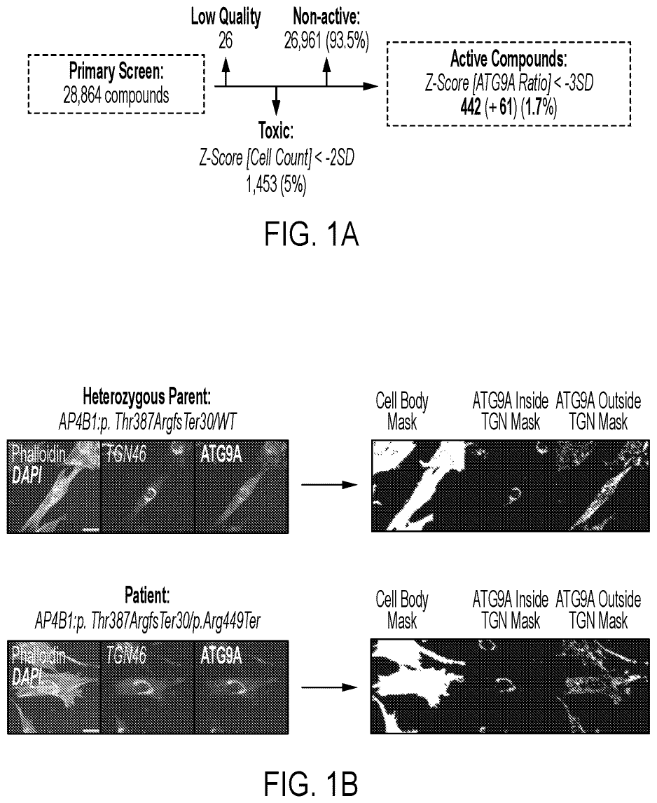

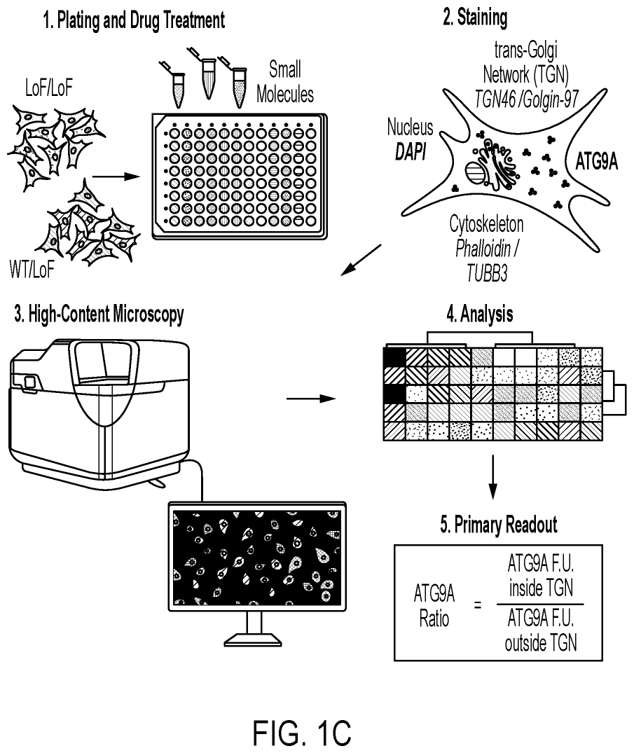

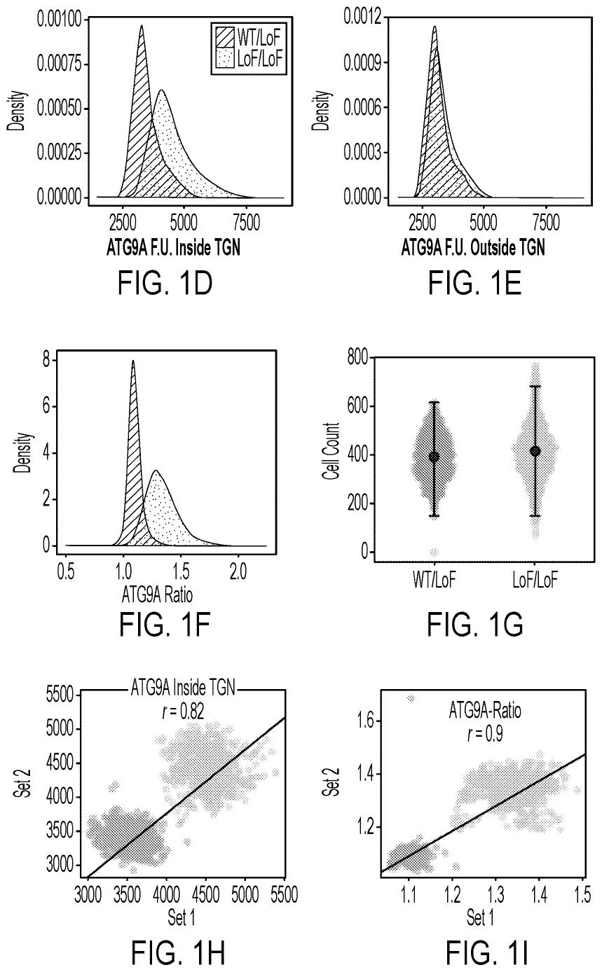

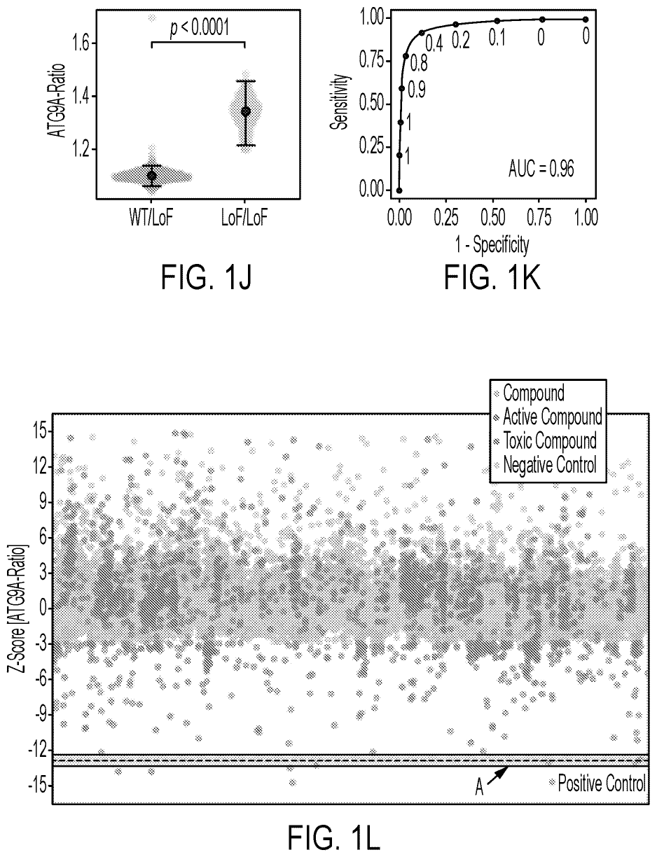

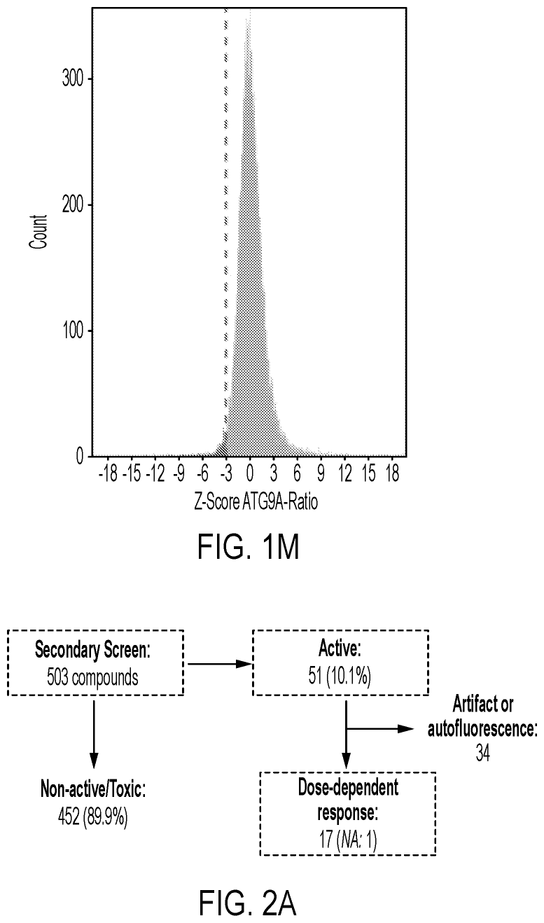

A diversity library of 28,864 novel small molecules was arrayed in 384-well microplates. The primary screening was conducted in fibroblasts from a well-characterized patient with all core features of SPG47 and bi-allelic loss-of-function variants in AP4B1 (NM_001253852.3: c.1160_1161del (p.Thr387ArgfsTer30)/c.1345A>T (p.Arg449Ter)) (FIGS. 1A-1B). Fibroblasts from the sex-matched parent (unaffected heterozygous carrier) served as controls. The assay was fully automated, miniaturized to 384-well microplates, and compounds were added for 24 hours at a single concentration of 10 μM (FIG. 1C).

The ATG9A ratio (ATG9A fluorescence intensity inside the TGN vs. in the cytoplasm) was used as the primary assay metric. The population distributions of the subcellular ATG9A signal inside and outside the TGN, at the level of single cells for negative (bi-allelic loss-of-function, LoF/LoF) and positive (heterozygous carriers, WT/LoF) controls are shown in FIG. 1D and FIG. 1E. The ATG9A ratio demonstrated a normal distribution and robust separation of both groups (FIG. 1F). Cell counts were similar for positive and negative controls, excluding cell death or changes in proliferation rates as possible confounding factors (FIG. 1G). To test for reproducibility across replicates, assay plates were randomly sampled into two sets, and similar positions on the assay plates were plotted against each other (FIGS. 1H-1I). Random sampling was simulated 100 times, and mean covariances using the Pearson correlation coefficient were calculated. Using the ATG9A ratio (FIG. 1I) as a primary readout resulted in higher replicate correlation (mean r=0.90±0.002 SD), compared to absolute ATG9A intensities (FIG. 1H) (mean r=0.82±0.0008 SD). ATG9A ratios showed robust discriminative power between positive and negative controls (LoF/LoF mean: 1.1±0.02 SD, n=1312 wells vs. WT/LoF mean: 1.34±0.05 SD, n=1312 wells; T-Test, p<0.0001) (FIG. 1J). The ATG9A ratio as the primary outcome metric was further supported by a generalized linear model, which demonstrated high specificity and sensitivity (FIG. 1K, AUC: 0.96).

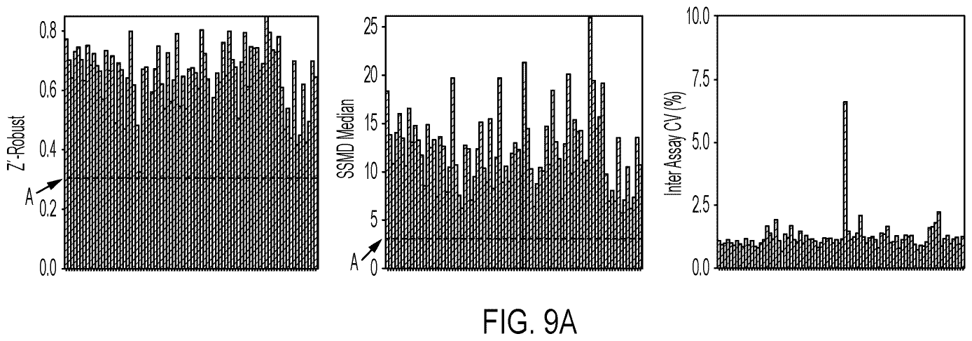

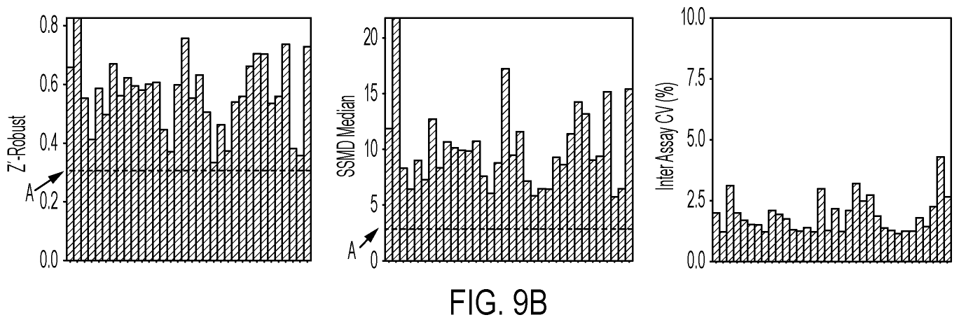

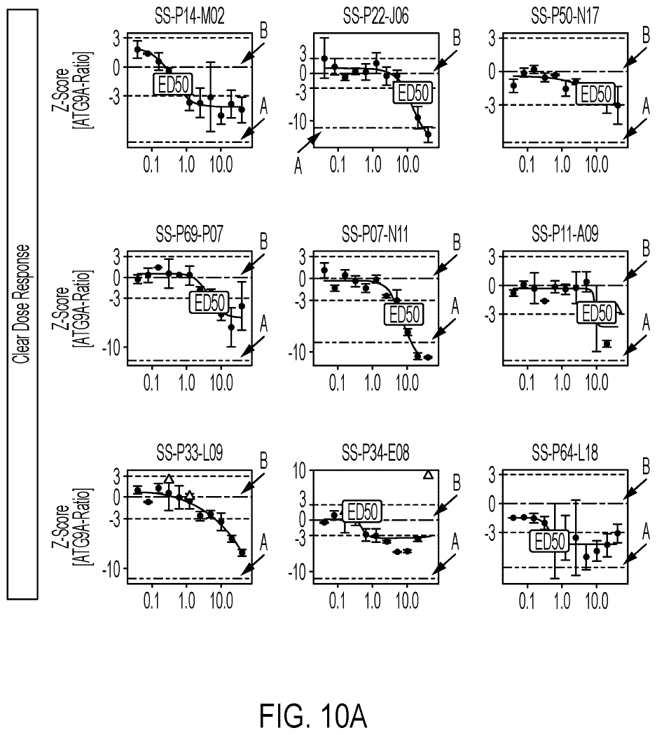

During the screening, assay performance was monitored using established quality control metrics for cell-based screens (Z′ robust ≥0.3, strictly standardized median difference ≥3, and an inter-assay coefficient of variation ≤10%). All assay metrics were calculated for positive and negative controls of the same assay plate to avoid bias by inter-plate variability. Predefined thresholds were met by all assay plates (FIG. 9A). The results of the primary screen are summarized in FIG. 1L and FIG. 1M.

Of the 28,864 compounds, 26 were excluded due to non-quantifiable ATG9A signal, based on low cell counts or imaging artifacts. The remaining 28,838 compounds were subsequently evaluated for changes in cell count and the ATG9A ratio. The vast majority of compounds (n=26,961, 93.5%) did not show any significant reduction in the ATG9A ratio (defined as a reduction by at least 3 SD). 1,435 (5.0%) compounds were excluded due to toxicity, defined as a reduction in the mean cell count by at least 2 SD compared to the negative controls. Only a small subset of 503 compounds (1.7%) reduced the ATG9A ratio by 3 or more SD compared to negative controls (FIG. 1M). Of these, 61 (0.2%) also reduced cell counts, while the remaining 442 (1.5%) showed no toxicity.

In summary, from this high-throughput primary screen, 503 active compounds were identified and 442 were selected for further testing.

Counter-Screen in Fibroblasts from AP-4-HSP Patients Confirmed 16 Compounds that Lead to a Dose-Dependent Redistribution of ATG9A

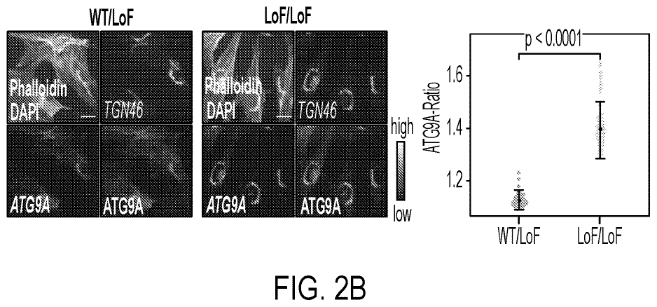

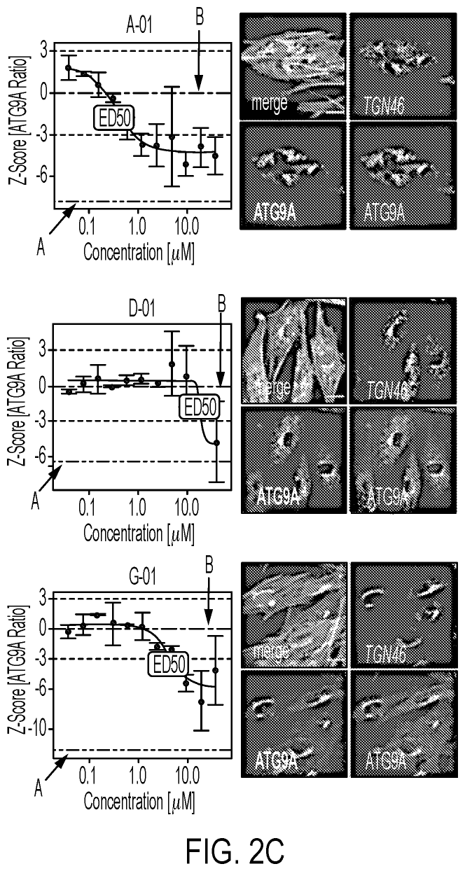

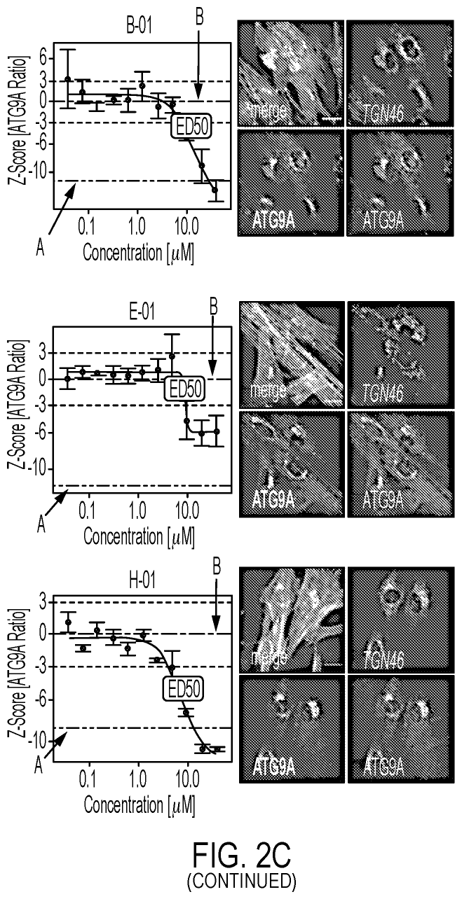

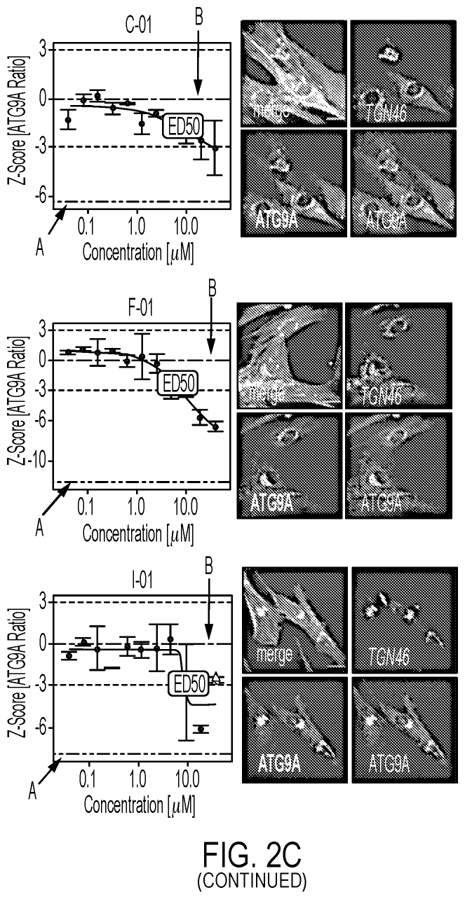

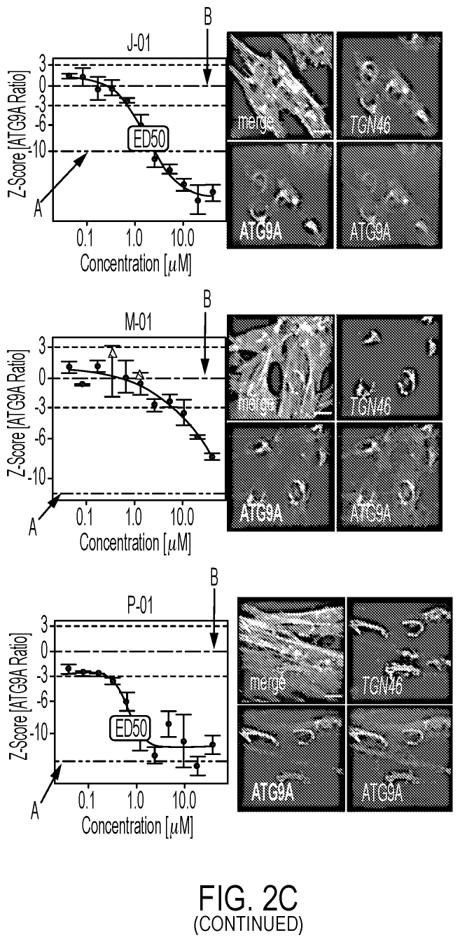

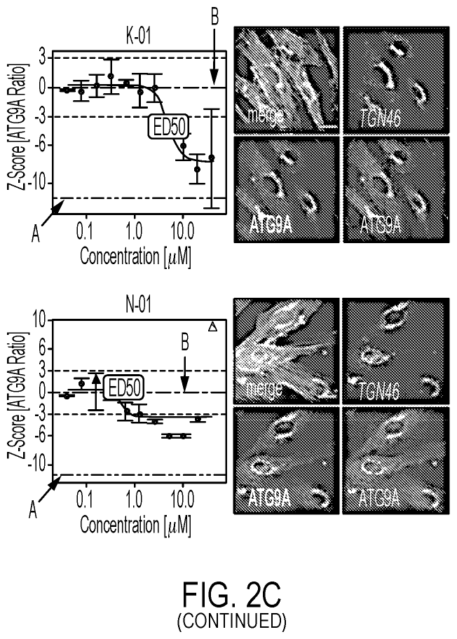

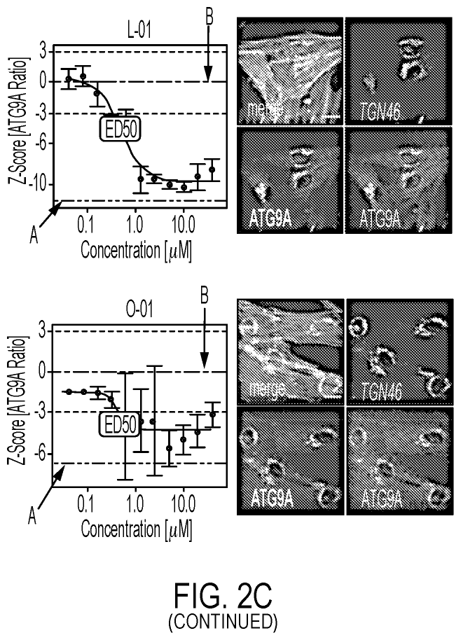

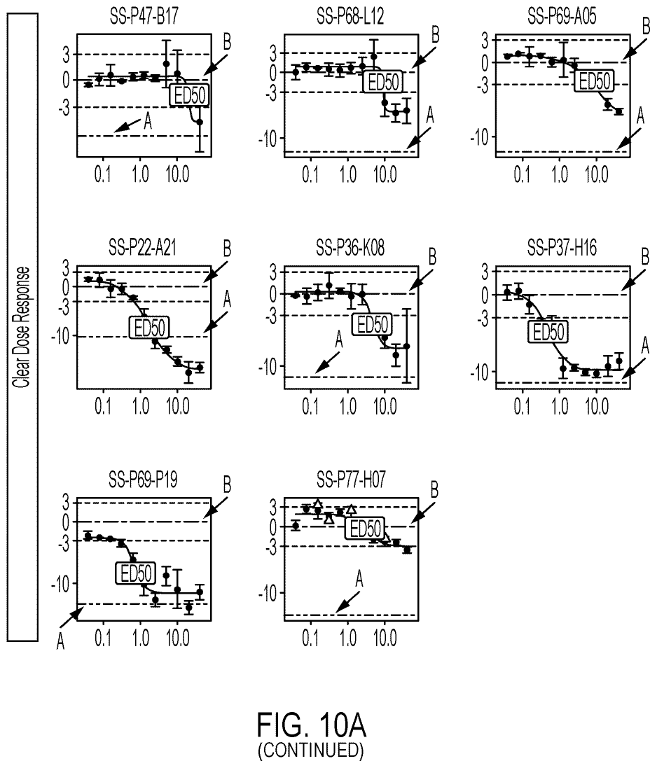

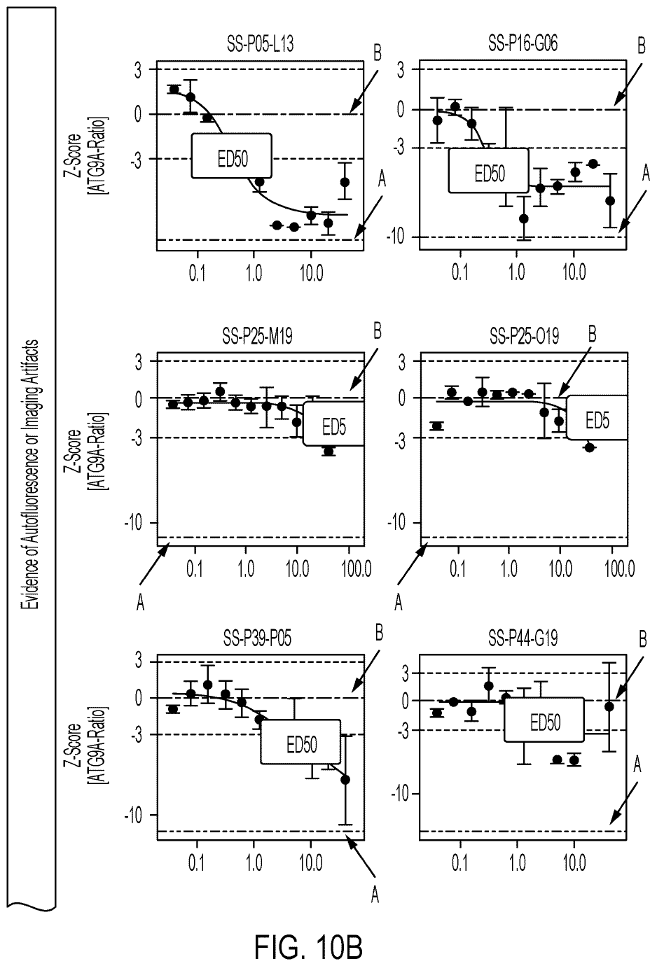

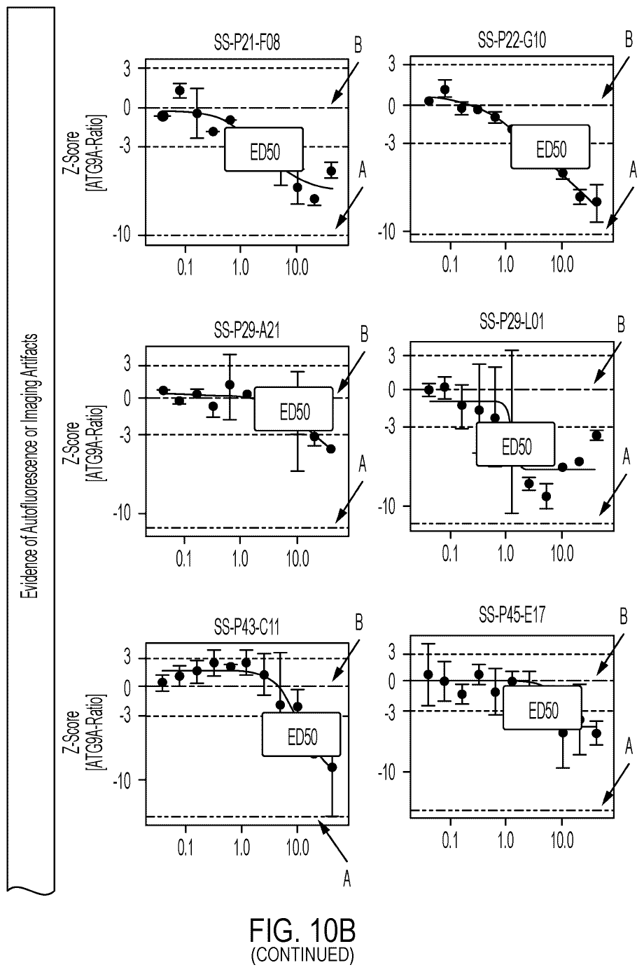

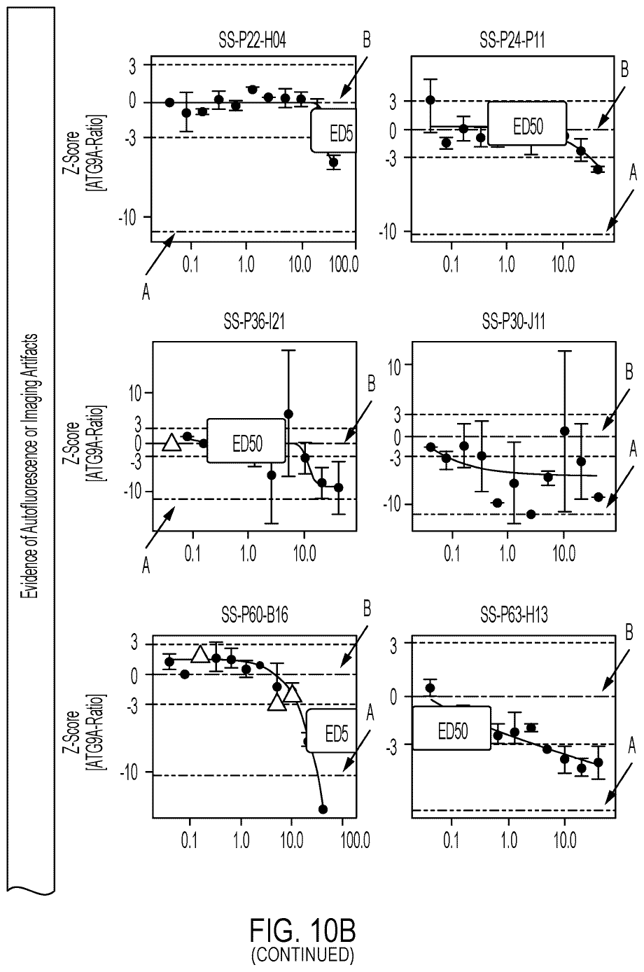

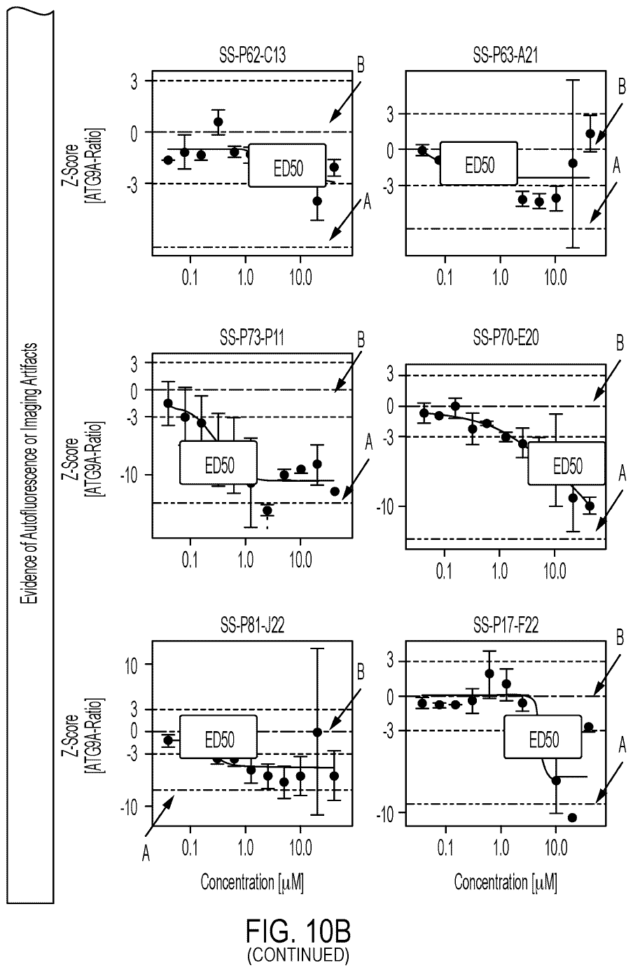

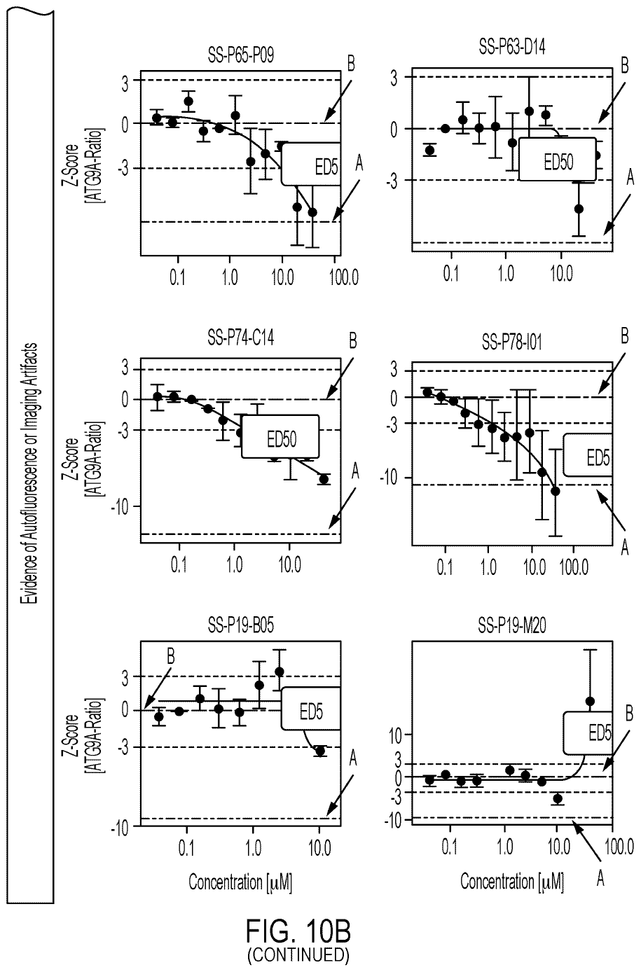

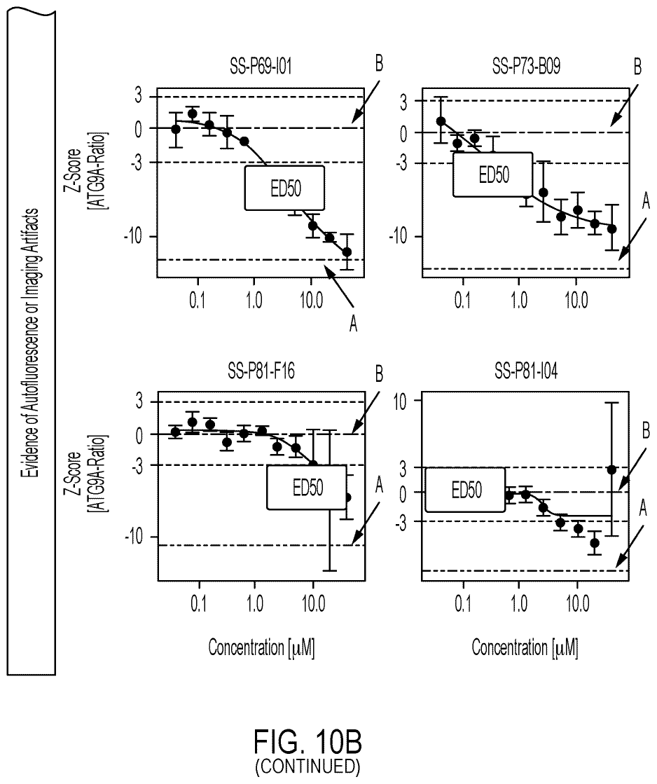

To confirm and further evaluate the 503 active compounds identified in the primary screen, compounds were retested for dose-dependency using an 11-point dose range (range: 40 nM to 40 μM) (FIG. 2A). All concentrations were screened in biological duplicates and subjected to the same quality control metrics as in the primary screen (FIG. 9B). Similar to the results from the primary screen, ATG9A ratios for negative and positive controls showed a robust separation (LoF/LoF mean: 1.4±0.07 (SD), n=269 wells, vs. WT/LoF mean: 1.12±0.02 (SD), n=269 wells, T-Test, p<0.0001, FIG. 2B). Activity in the secondary screen was defined as the ability to reduce the ATG9A ratio by at least 3 SD in both replicates and at least 2 different concentrations, without exerting toxicity. 51 compounds (10.1%) met these a priori defined criteria (FIGS. 10A-10B). After manually verifying image quality and validating dose-response relationships, compounds were triaged (FIG. 2A and FIGS. 10A-10B). Seventeen compounds demonstrated a clear and reproducible dose-response relationship, without evidence of image artifacts or autofluorescence. The ED50 for most compounds were in the low micromolar range (median: 4.66 μM, IQR: 8.63, FIG. 2). 34 compounds were found to carry autofluorescence or imaging artifacts and were thus excluded from further testing (FIG. 10B).

In summary, a counter-screen in AP-4-deficient patient fibroblasts confirmed and established dose-dependent effects on the intracellular ATG9A distribution for 16 compounds (FIG. 2C).

### Orthogonal Assays in Neuronal Models of AP-4-Deficiency Confirmed 5 Active Compounds

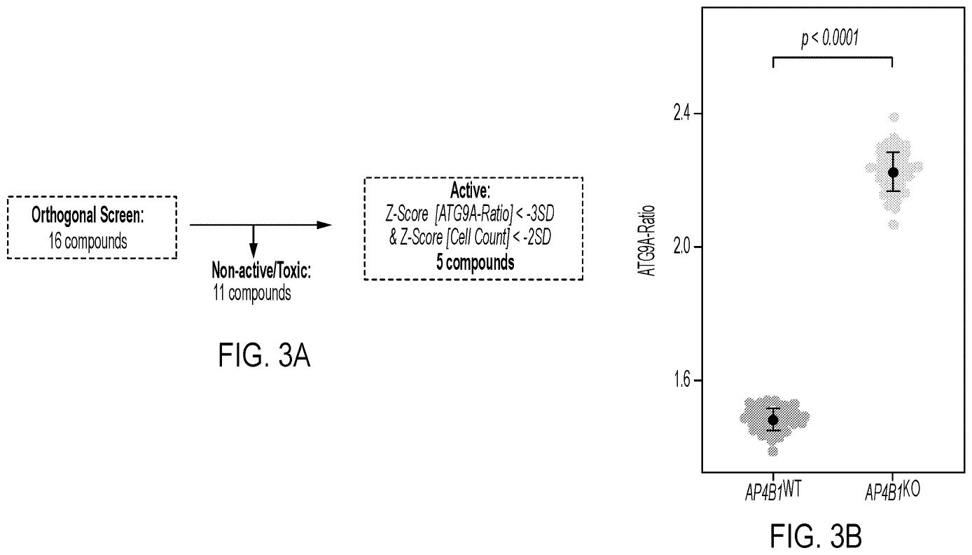

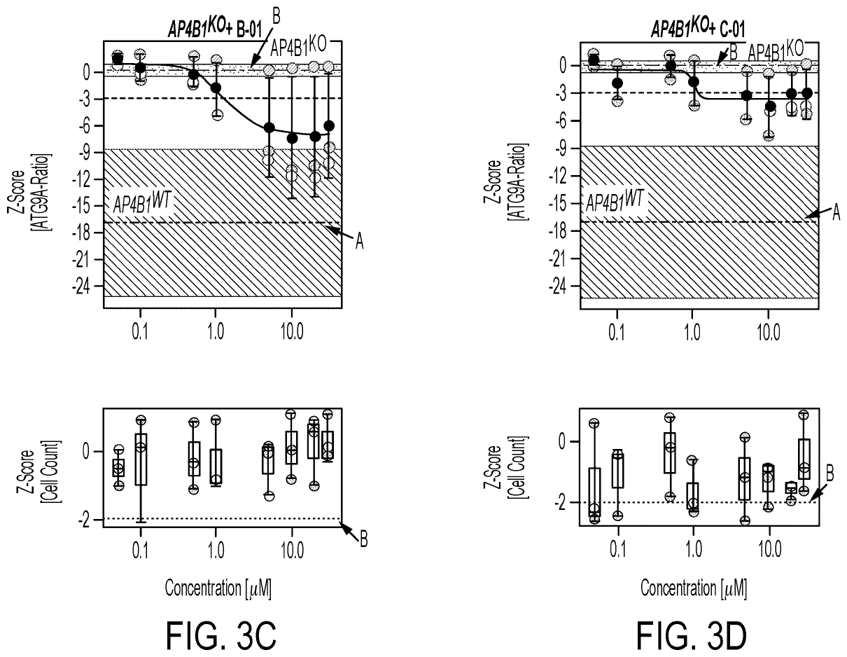

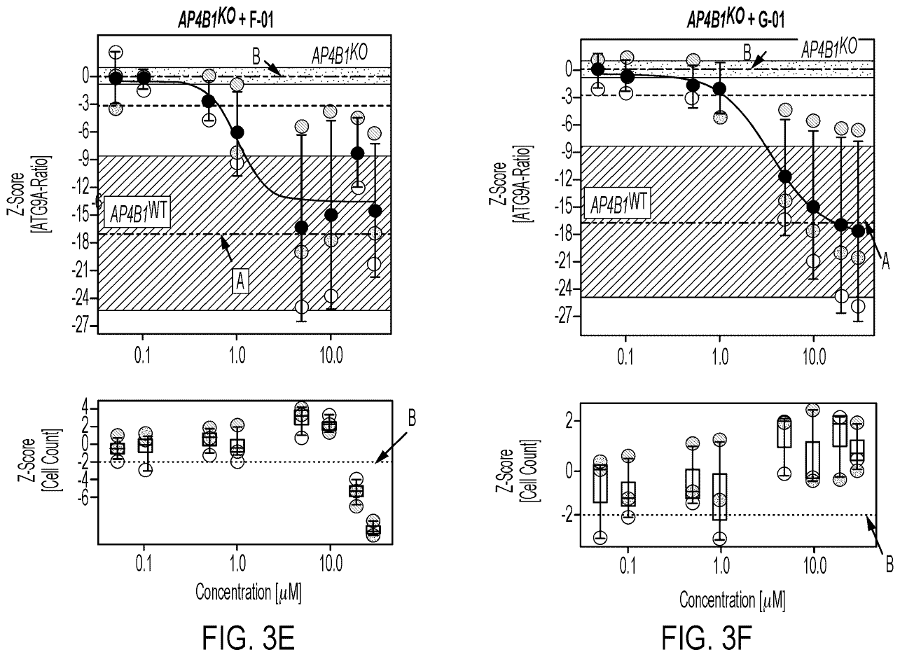

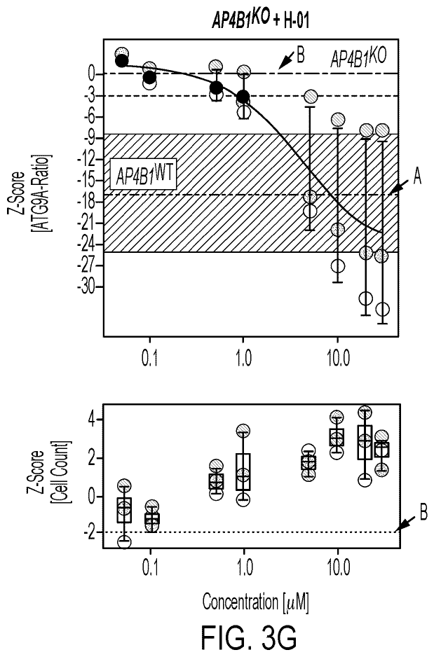

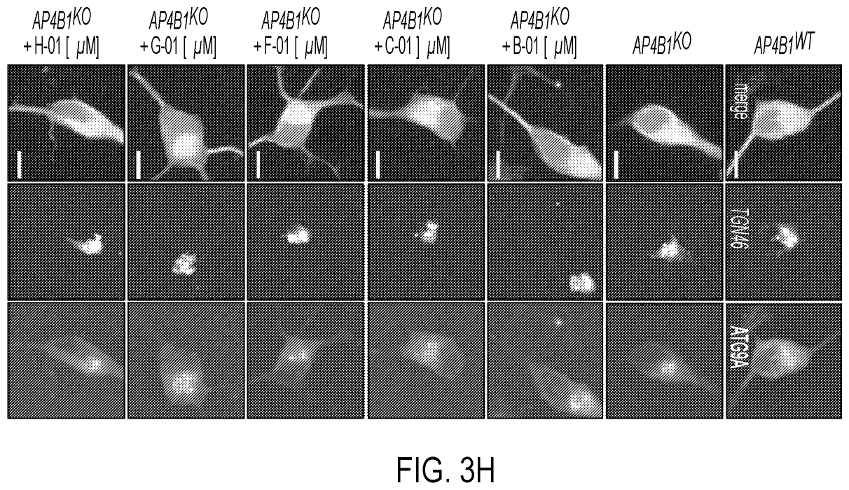

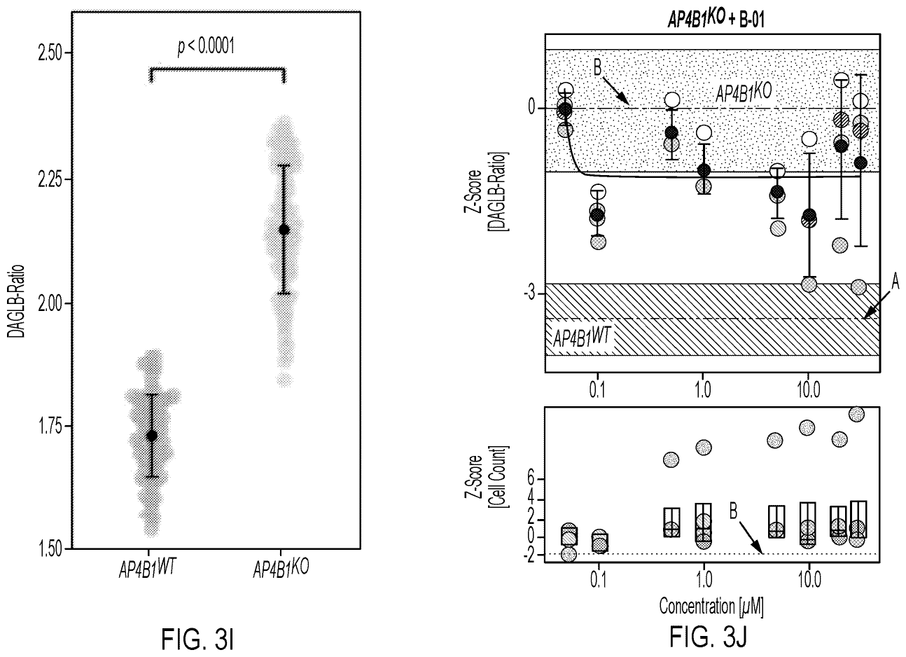

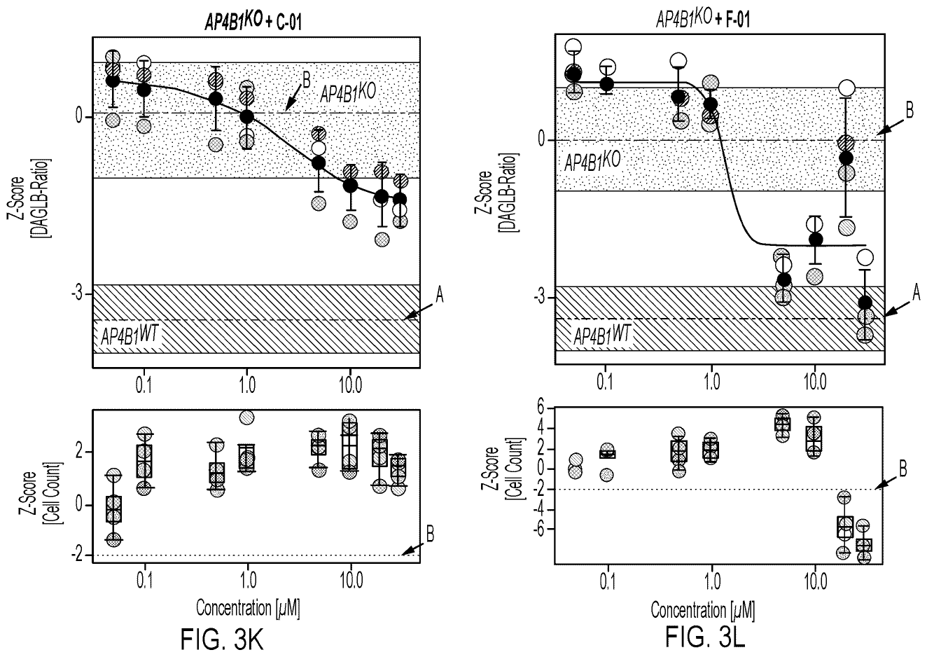

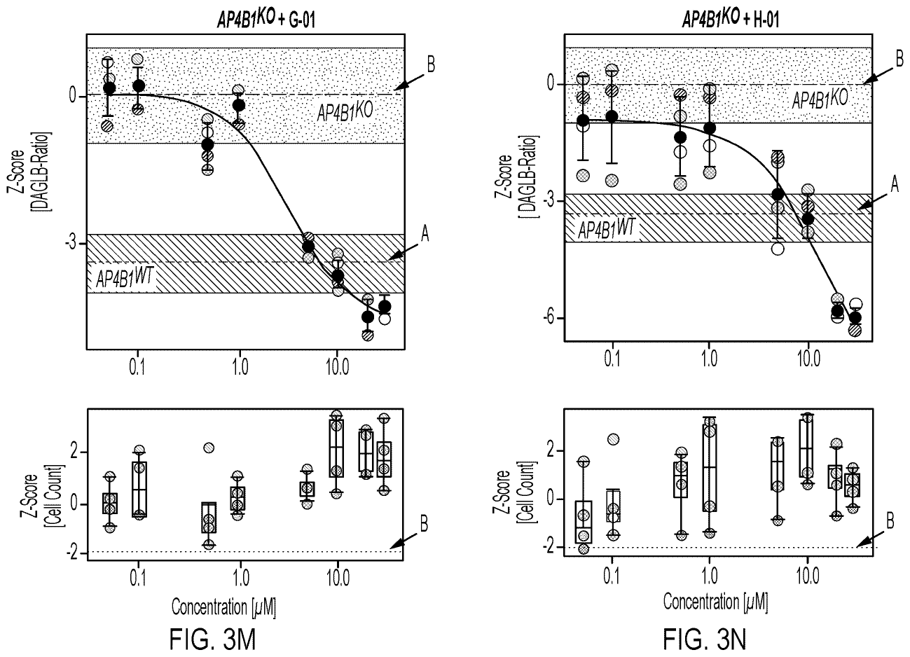

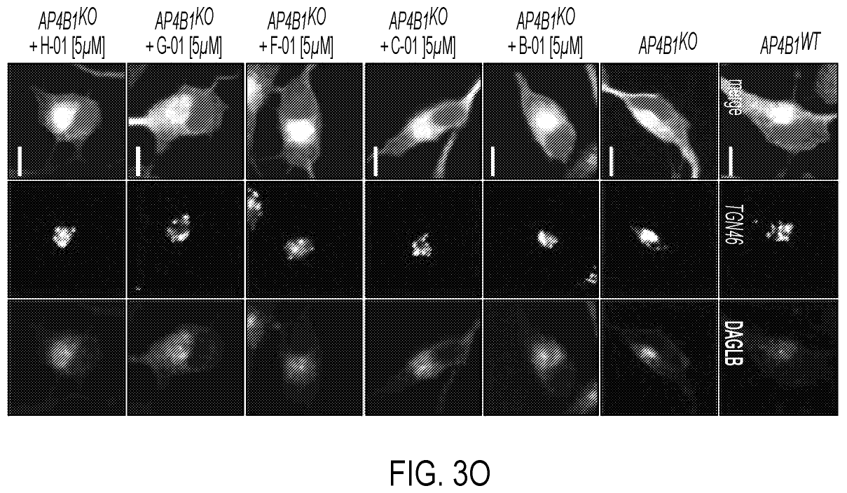

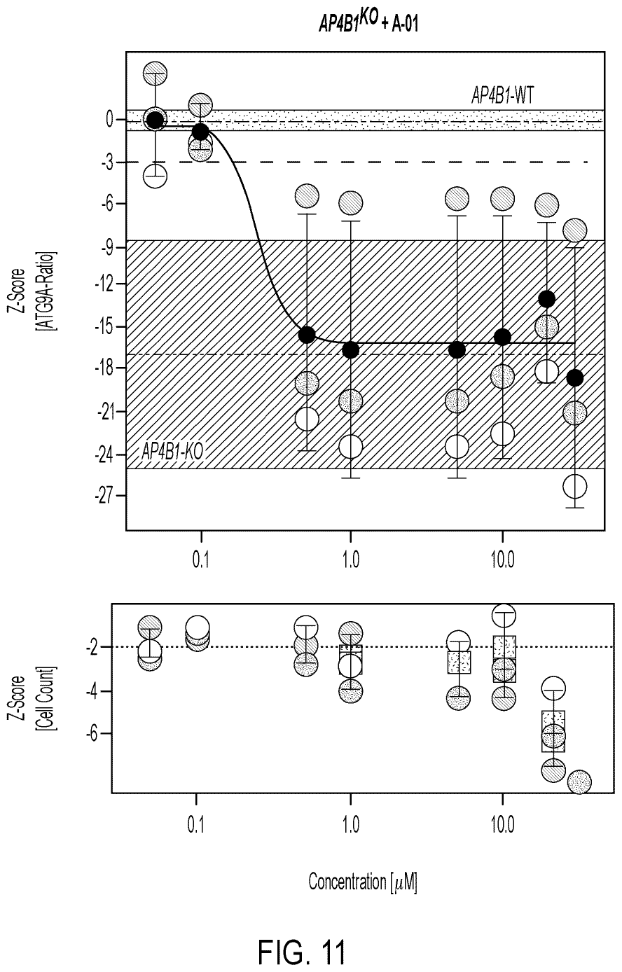

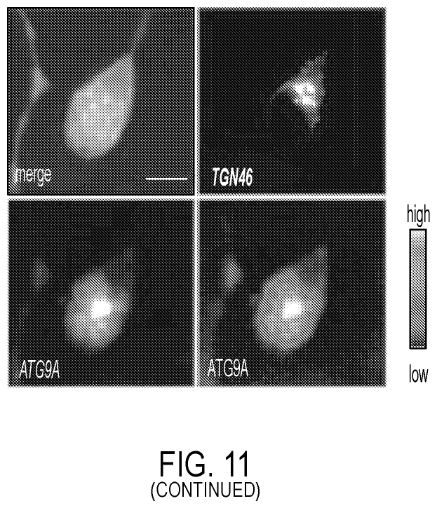

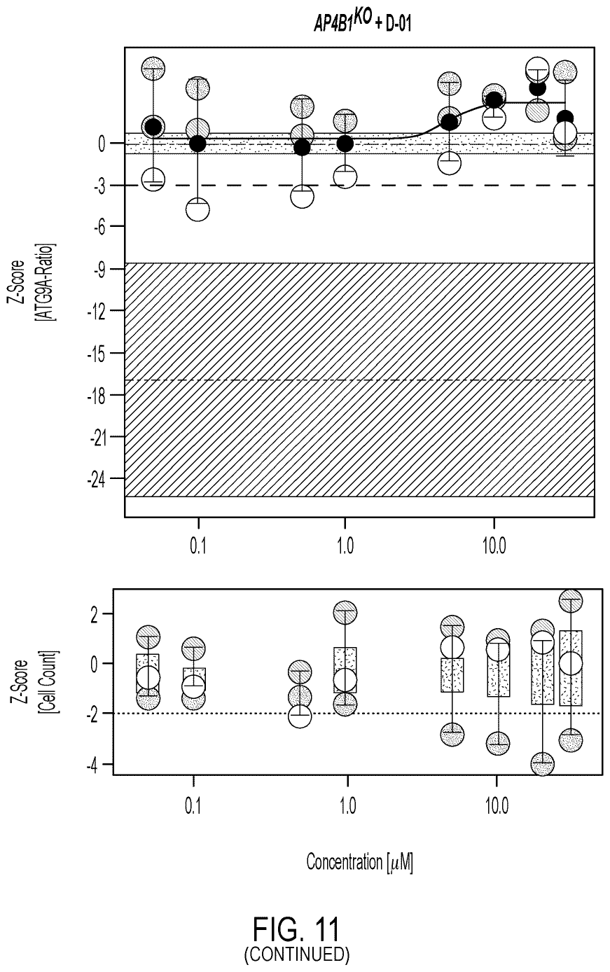

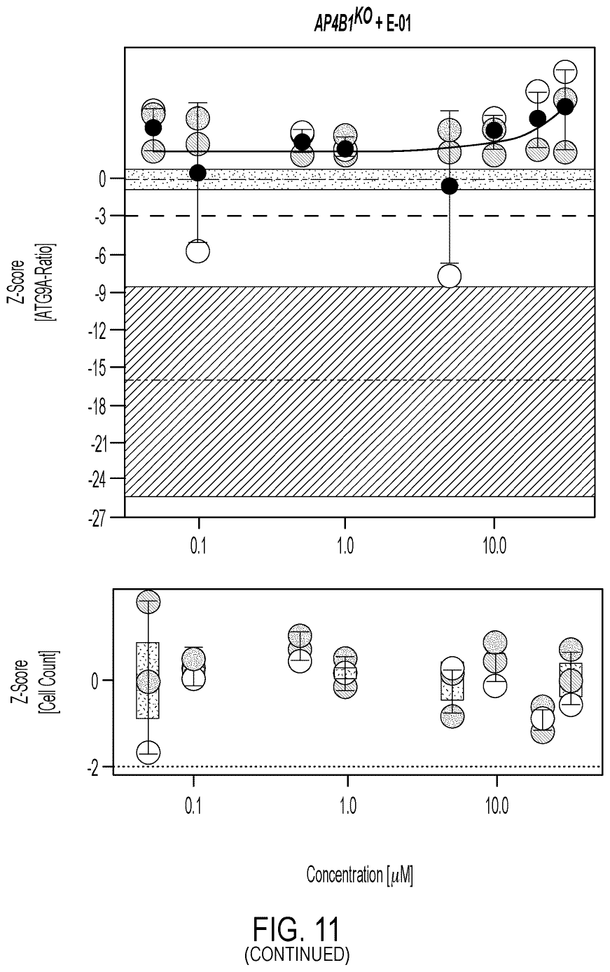

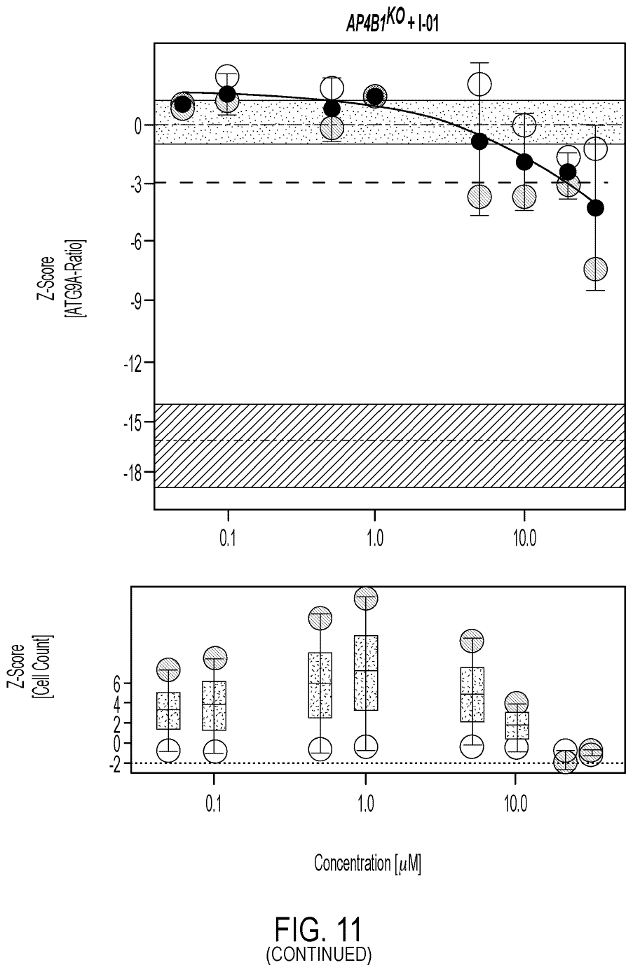

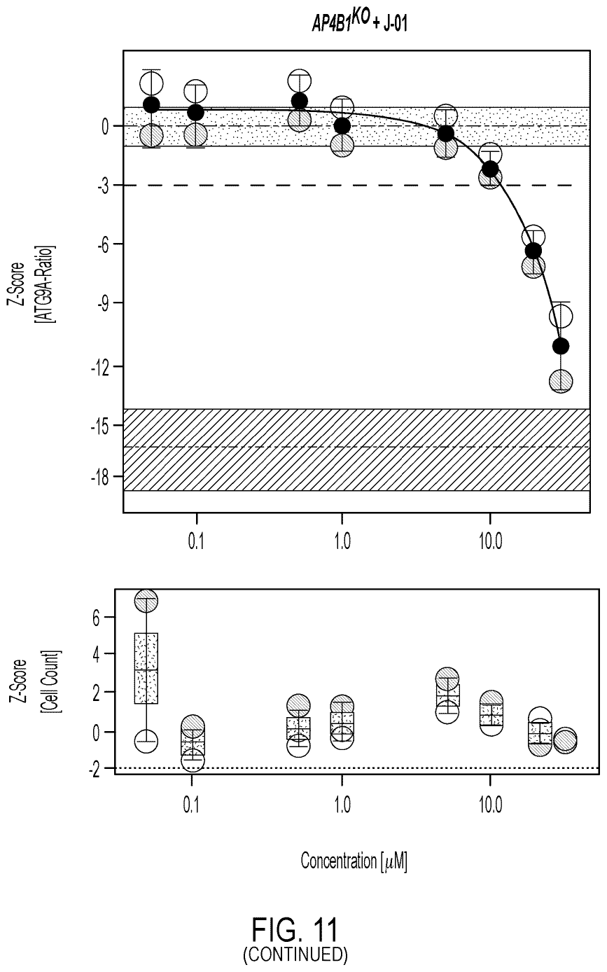

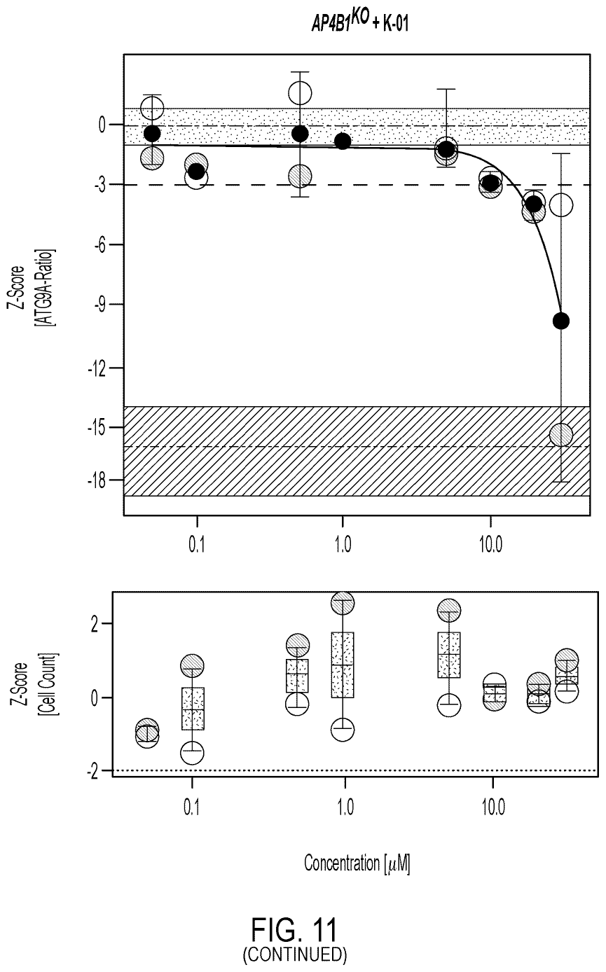

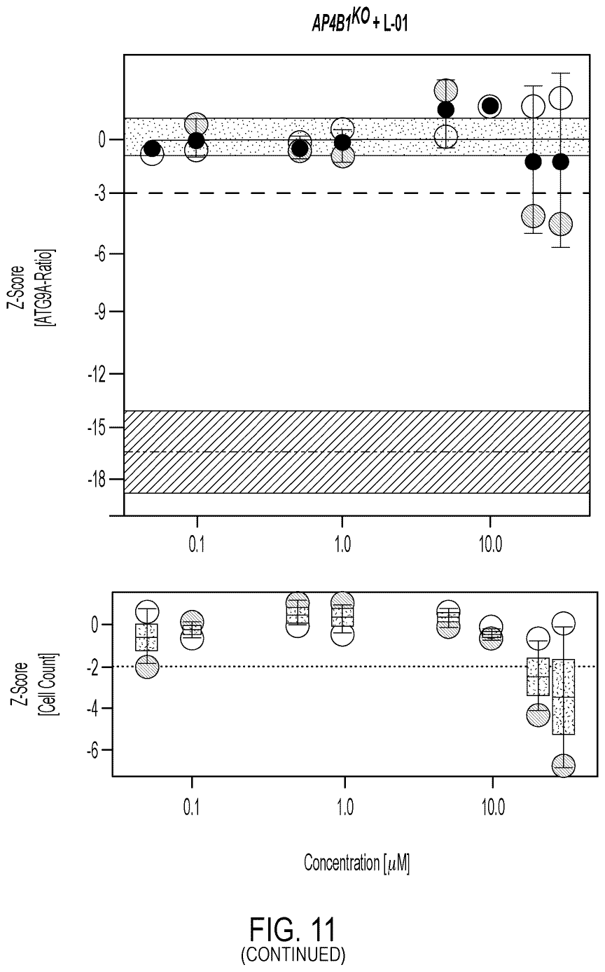

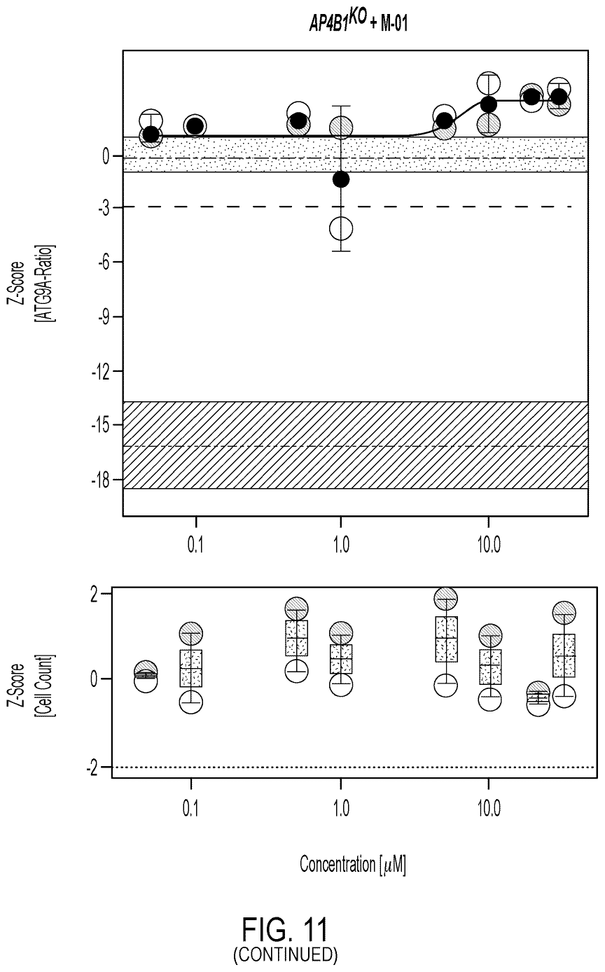

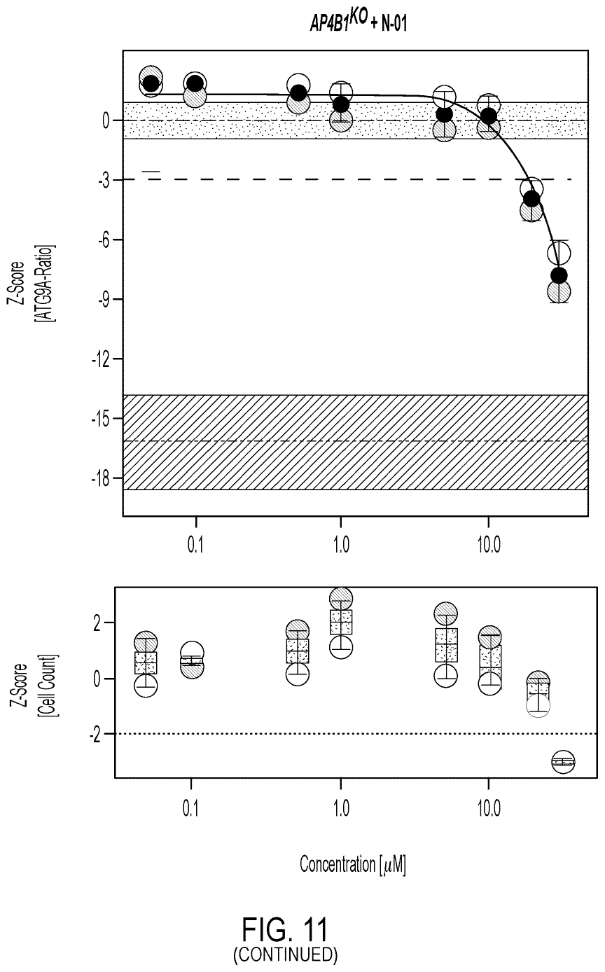

To validate active compounds from the secondary screen in a human cell line with neuron-like properties, the ATG9A assay was optimized for neuroblastoma-derived SH-SY5Y cells following a 5-day neuronal differentiation protocol with retinoic acid (FIG. 3A). SH-SY5Y cells with a stable AP4B1-knockout (AP4B1^{KO}) served as negative controls while AP4B1-wildtype (AP4B1^{WT}) cells were used as positive controls. All 16 active compounds were tested in 8-point dilutions (range: 50 nM to 30 μM) with a treatment duration of 24 hours. Quantification of the ATG9A ratio in differentiated SH-SY5Y cells showed a robust separation between control conditions (AP4B1^{KO}: 1.80±0.06 (SD), n=158 wells vs. AP4B1^{T}: 1.17±0.03 (SD), n=160 wells, T-Test, p<0.0001, FIG. 3B). Compounds were evaluated based on their dose-dependent reduction of the ATG9A ratio and absence of cell toxicity. Eleven of 16 compounds were excluded due to lacking activity (n=7), suspicion for artefacts or autofluorescence (n=3), or obvious changes in cellular morphology (n=1) (FIG. 11). Of the five remaining compounds, three restored the ATG9A ratio to levels of wildtype controls (F-01, G-01 and H-01) while two compounds (B-01 and C-01) led to a reduction by at least 3 SD at higher concentrations (FIGS. 3C-3H).

To assess whether these effects were specific to ATG9A or similar effects were also present for other AP-4 cargo proteins, a second neuronal AP-4 cargo protein, DAGLB, was used. Similar to ATG9A, the DAGLB ratio (DAGLB fluorescence intensity in the TGN vs. in the cytoplasm), showed a robust separation between AP4B1^{WT} and AP4B1^{KO} cells (AP4B1^{KO}: 1.80±0.1 (SD), n=192 wells vs. AP4B1^{WT}: 1.36±0.07 (SD), n=192 wells, T-Test, p<0.0001, FIG. 3I). All five active compounds showed activity in the DAGLB assay, suggesting a broader effect on the trafficking of at least 2 AP-4 cargo proteins from the TGN. Again, F-01, G-01 and H-01 (FIGS. 3L-3O) resulted in normalization of the intracellular DAGLB distribution, while B-01 and C-01 led to a moderate reduction of DAGLB ratios at higher concentrations (FIGS. 3J-3K, FIG. 3O).

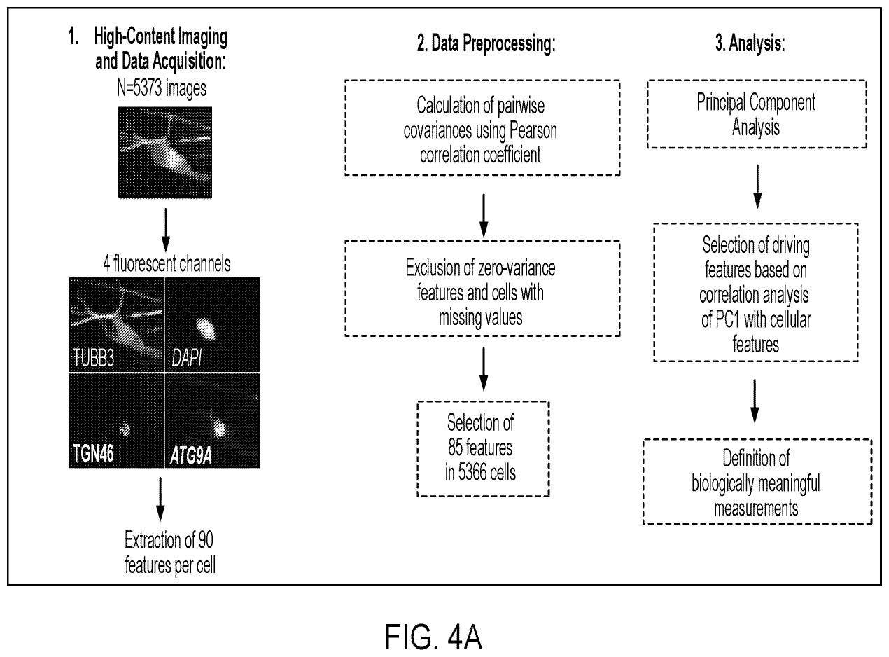

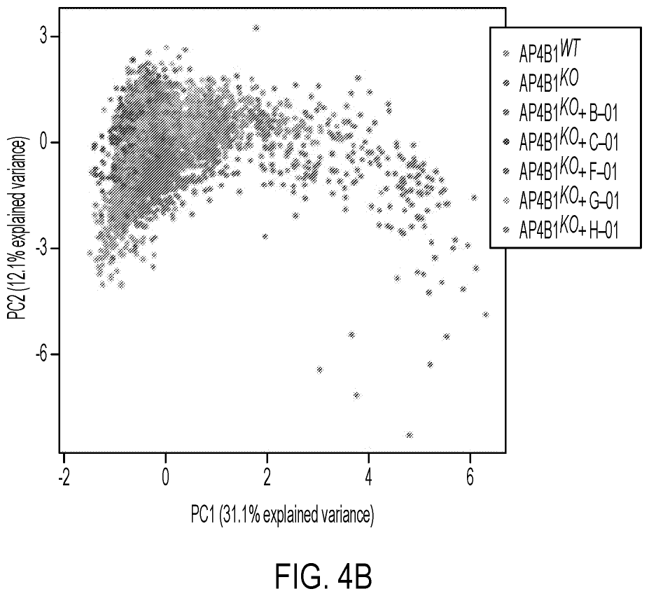

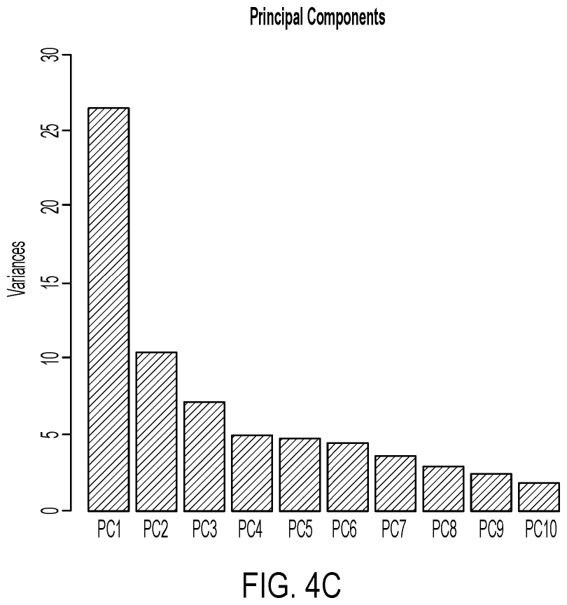

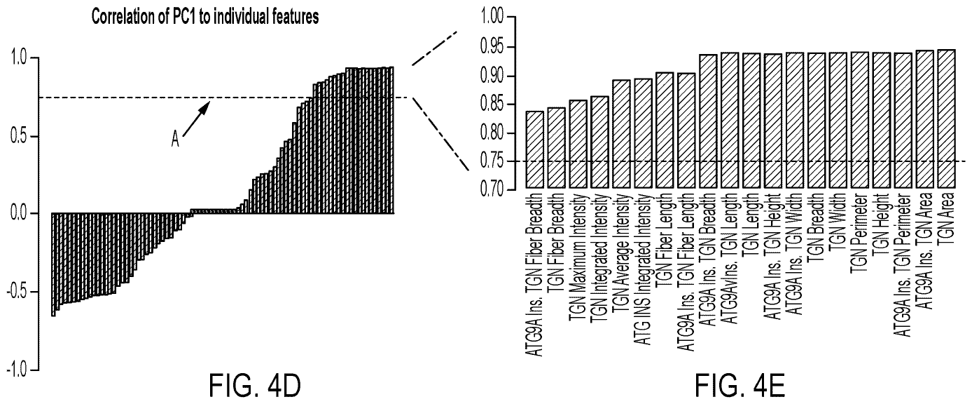

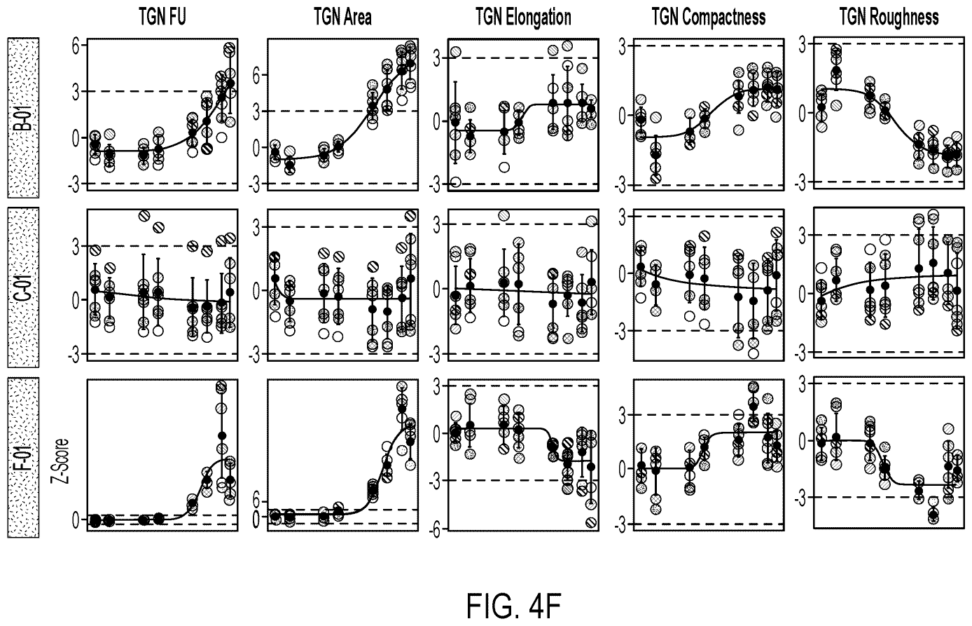

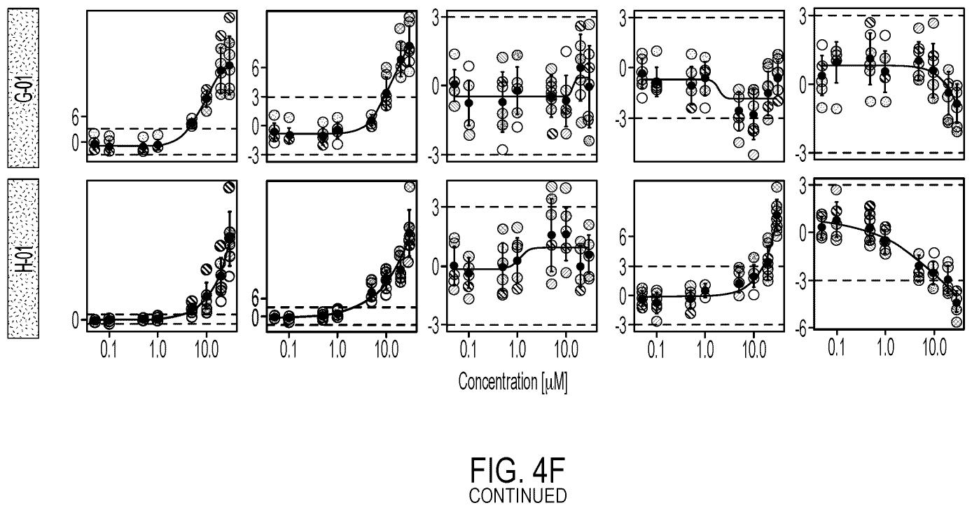

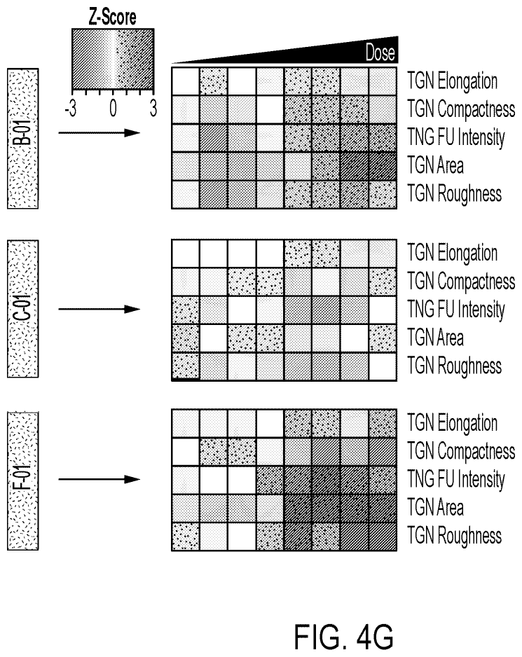

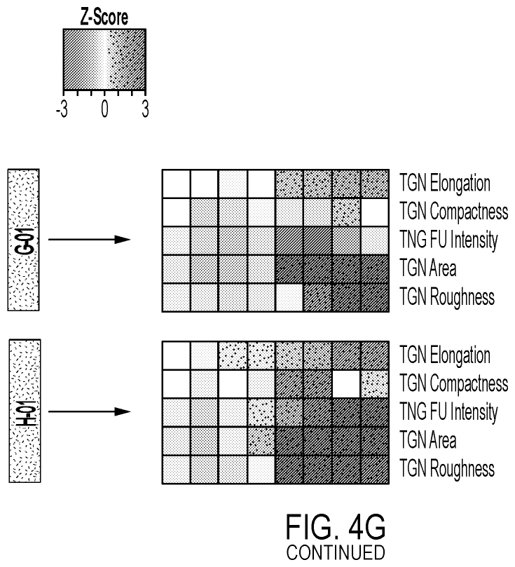

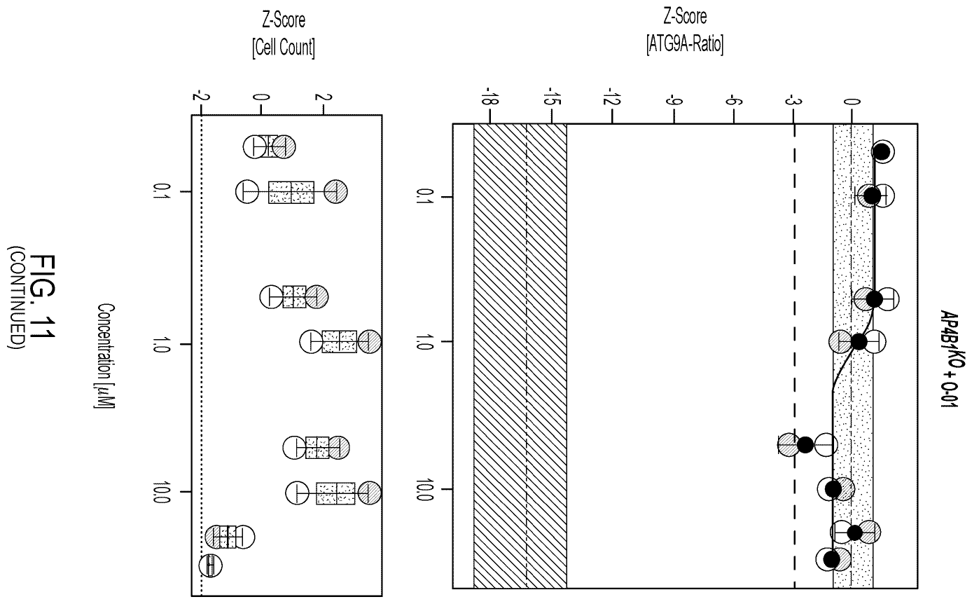

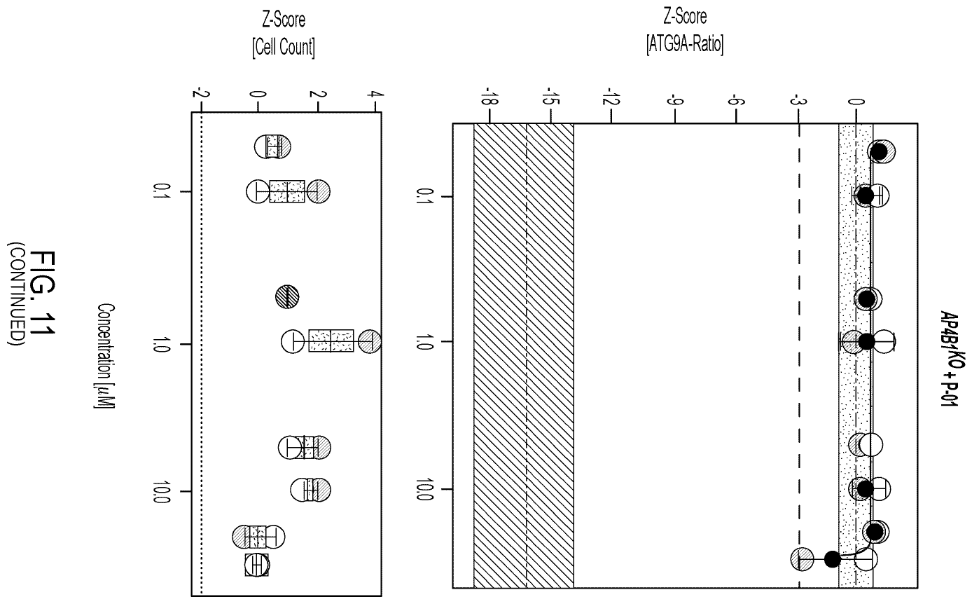

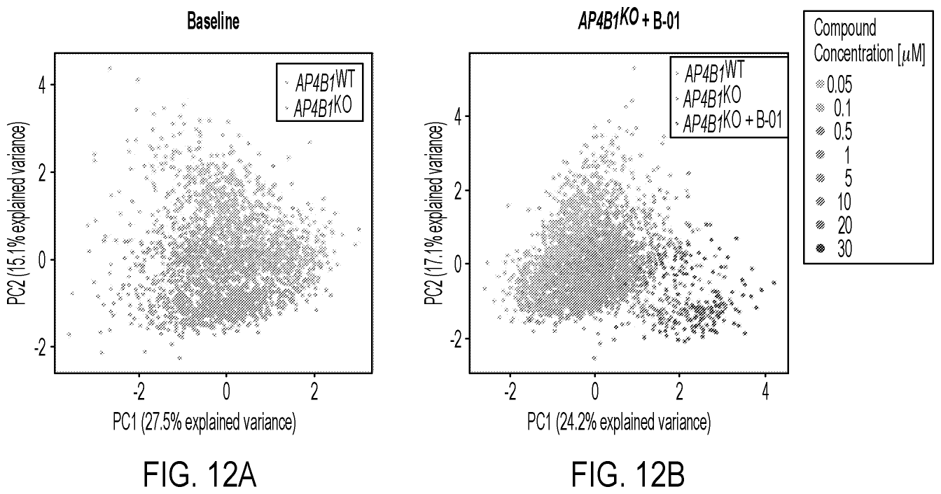

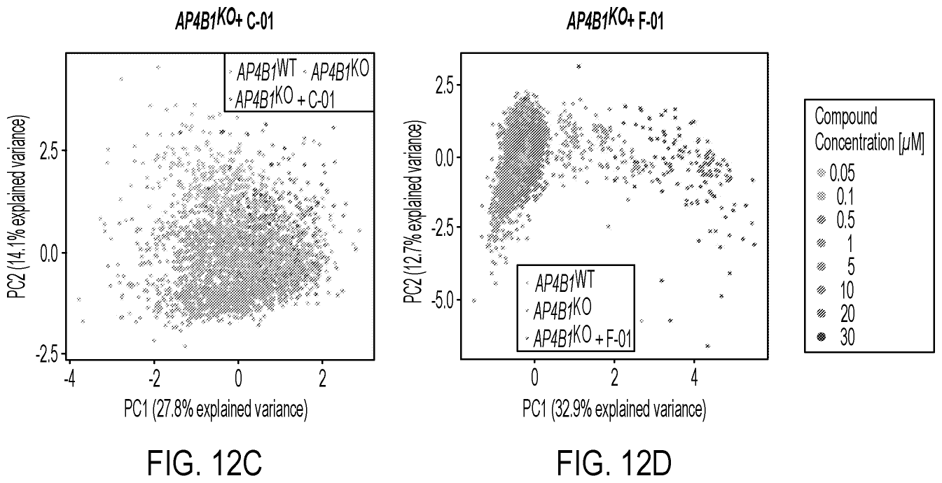

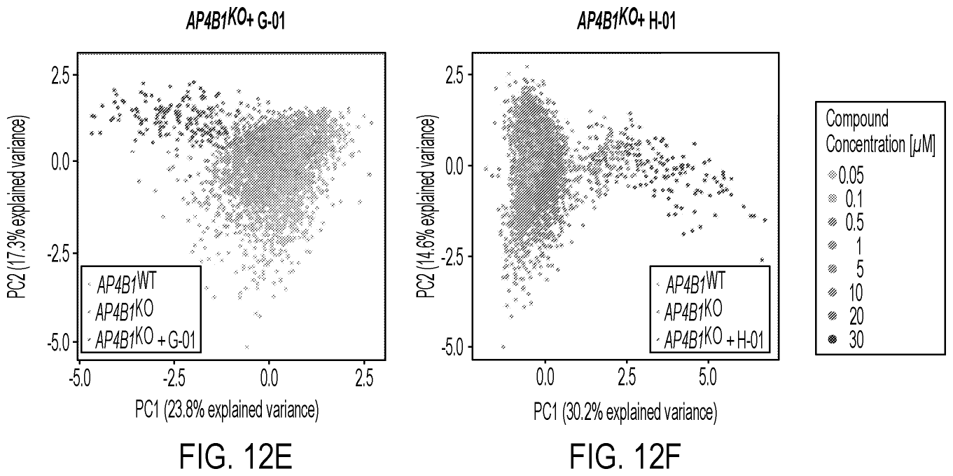

Since small molecules can have pleotropic effects on cellular functions and organellar morphology, next a multiparametric morphological profiling approach was employed. Eighty-five measurements of the nucleus, cytoskeleton, global cell morphology, the TGN and ATG9A vesicles were automatically computed for each image, serving as a rich and unbiased source for interrogating biological perturbations induced by compound treatment. Principal component analysis was used to reduce dimensionality and cluster images based on their properties (FIG. 4A and FIG. 12). Positive and negative controls clustered closely together and were separated only by the ATG9A signal (FIG. 4B and FIG. 12A). B-01, C-01 and G-01 showed properties comparable to positive and negative controls, suggesting little off-target effects (FIG. 4B, FIGS. 12B-12C, FIG. 12E). F-01 and H-01, however, changed cellular morphology in a dose-dependent manner (FIG. 4B and FIG. 12D, FIG. 12F), with changes mainly driven by the first principal component, accounting for 31.1% of the observed variance (FIG. 4C). To decipher the phenotypic alterations responsible for these changes, the covariances (Pearson correlation coefficient) of the first principal component with each measurement were calculated (FIG. 4D). Features with a correlation coefficient >0.75 were selected to define morphological profiles (FIG. 4E). TGN fluorescence intensity and morphology seemed to be the most significant drivers for the separation, suggesting that disruption of TGN integrity potentially biased the assessment of ATG9A ratios in cells treated with compounds F-01 and H-01 (FIG. 4B and FIG. 12D, FIG. 12F).

Following these analyses, TGN fluorescence intensity and morphological measures such as TGN area and elongation, as well as compactness and roughness, as indicators of the complexity of the TGN, were quantified for cells treated with all five active compounds (FIG. 4F-4G). While C-01 showed stable TGN signal and morphology across all assessed measurements, all other compounds depicted some degree of change. Again, F-01 and H-01 seemed to result in TGN changes in a dose-dependent manner while B-01 and G-01 led to only moderate alterations (FIG. 4F-4G). Of note, these changes to TGN morphology were undetectable by visual inspection but only delineated through an automated analysis of ~600 images per group, showcasing the power of the automated, unbiased, high-throughput platform.

C-01 Restored ATG9A and DAGLB Trafficking in iPSC-Derived Neurons from AP-4-HSP Patients

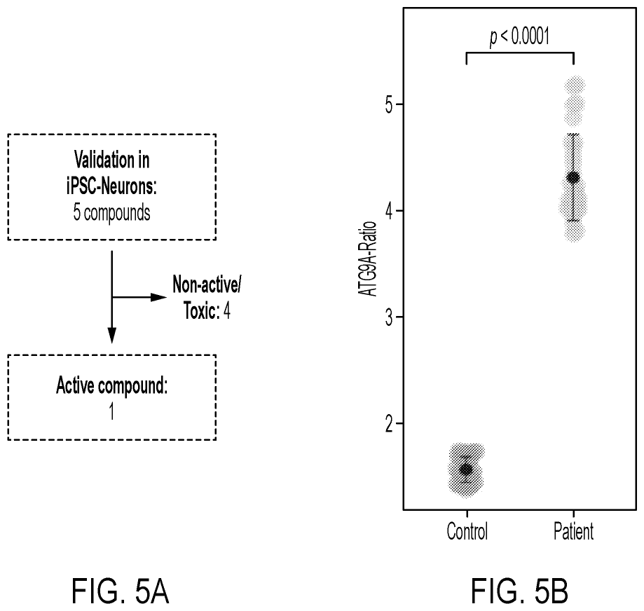

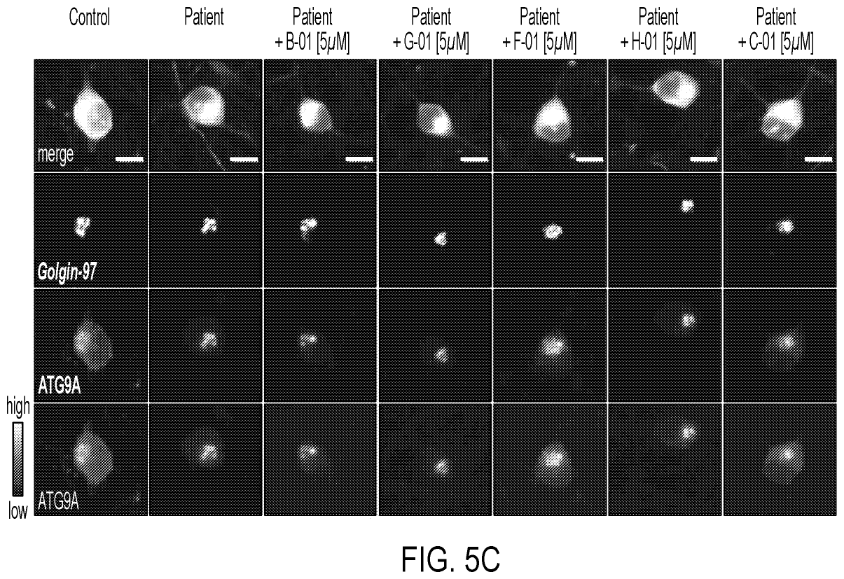

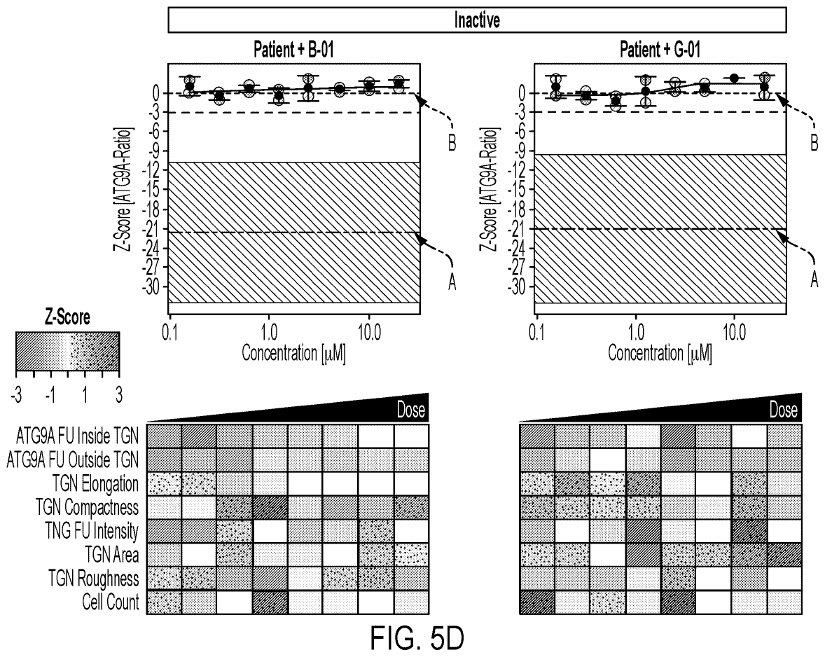

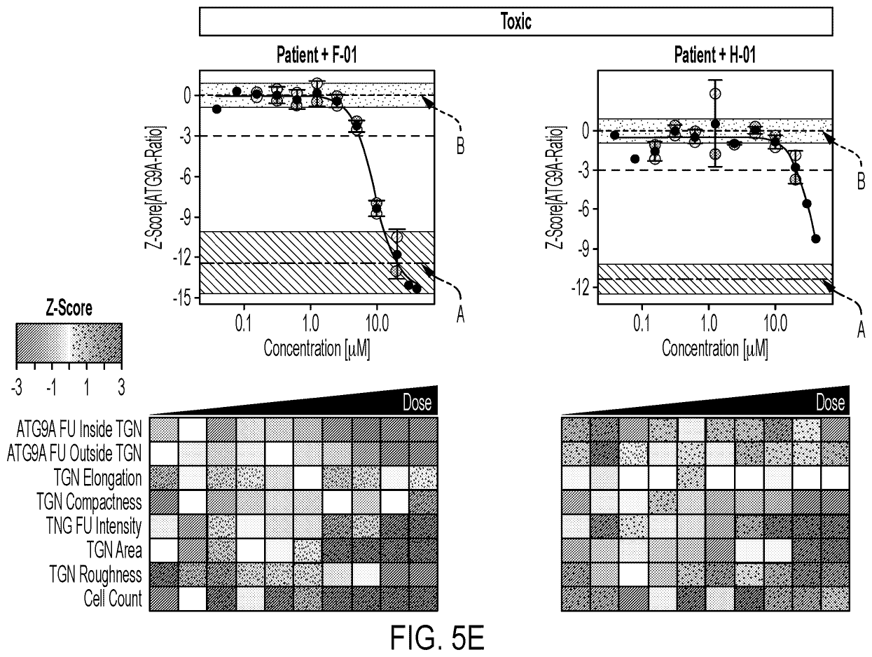

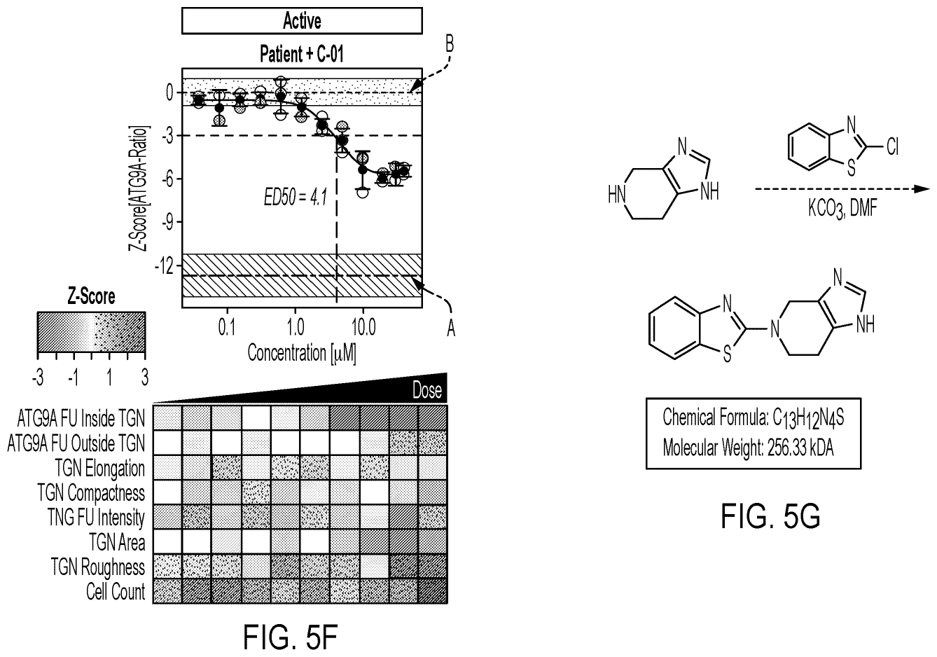

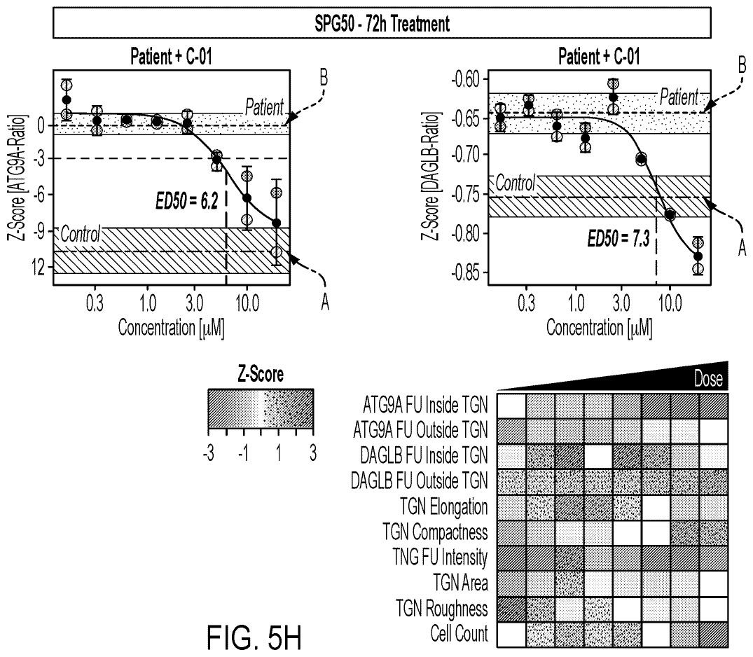

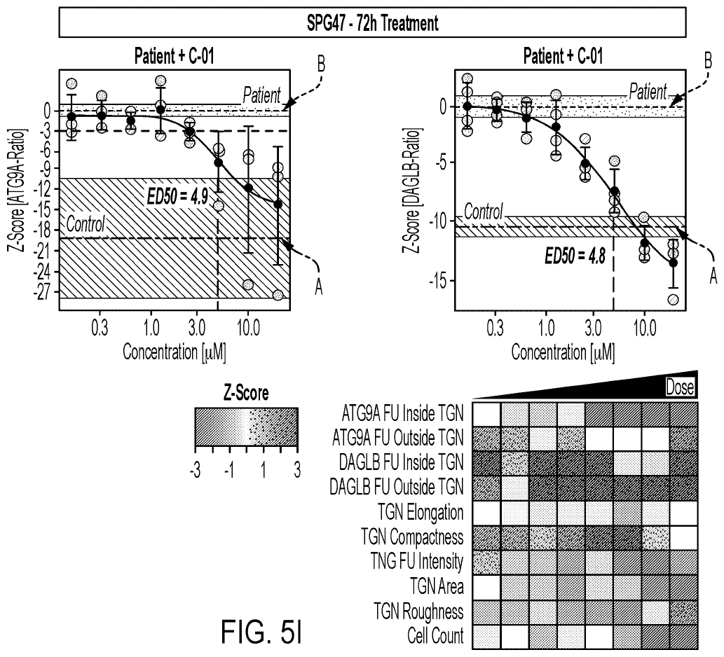

Informed by the findings in differentiated AP4B1^{KO} SH-SY5Y cells, these results were then investigated to determine if they would translate to human neurons. iPSCs from patients with AP-4-HSP due to biallelic loss-of-function variants in AP4M1 (NM_004722.4: c.916C>T (p.Arg306Ter)/c.694dupG (p.Glu232GlyfsTer21)) and AP4B1 (NM_001253852.3: c.1160_1161del (p.Thr387ArgfsTer30)/c.1345A>T (p.Arg449Ter)) were generated and differentiated into glutamatergic cortical neurons using established protocols. iPSC-derived neurons from sex-matched parents (unaffected heterozygous carriers) served as controls (FIG. 5A). Baseline quantification of ATG9A ratios in DIV14 neurons treated with vehicle for 24 hours showed robust separation between patient and control lines, exceeding the differences observed in AP-4-deficient fibroblasts and differentiated SH-SY5Y cells (SPG50 patient mean: 4.31±0.4 (SD), n=60 wells vs. heterozygous control: 1.56±0.12 (SD), n=60 wells, T-Test, p<0.0001, FIG. 5B). Neurons were treated for 24 hours in 8-point dose titration experiments. B-01 and G-01 lacked activity on the ATG9A ratio and were thus excluded (FIG. 5D). C-01, F-01 and H-01, by contrast, showed a robust reduction in the ATG9A ratio (FIGS. 5E-5F). A multiparametric analysis showed that, similar to observations in AP4B1^{KO} SH-SY5Y cells, only C-01 preserved TGN integrity (FIG. 5F) while F-01 and H-01 impacted TGN morphology, suggesting off-target effects (FIG. 5E). Based on its favorable profile, C-01 was re-synthesized for further testing (FIG. 5G). Orthogonal experiments, using prolonged treatment of C-01 for 72 hours to test for ATG9A and DAGLB translocation, demonstrated that C-01 was able to restore ratios of both AP-4 cargo proteins to levels close to controls with an ED50 of ~5 μM, while maintaining a favorable profile (FIG. 5H). This greater effect on ATG9A distribution, compared to the ~50% reduction of the ATG9A ratio at 24 hours treatment, suggests a time- and dose-dependent effect. C-01 changed the ATG9A ratio through decreasing ATG9A intensities inside the TGN while, at the same time, increasing cytoplasmic ATG9A levels, suggesting ATG9A translocation as the most likely mechanism of action. No changes in TGN morphology or any other cellular measurements were observed, indicating overall preservation of cellular morphology and little off-target effects. A similar pattern was observed with respect to DAGLB translocation (FIG. 5H). These findings were confirmed in a second set of experiments in iPSC-derived neurons from a patient with SPG47 (FIG. 5I), demonstrating that findings extend to other forms of AP-4-deficiency.

Taken together, C-01 emerged as a robust modulator of ATG9A and DAGLB trafficking in human neurons from patients with AP-4 deficiency.

### Target Deconvolution Using Transcriptomic and Proteomic Analyses Delineated Putative Mechanisms of Action for C-01

To explore potential mechanisms of action of C-01 in an unbiased manner, a multi-omics approach was used, combining bulk RNA sequencing and unbiased label-free quantitative proteomics.

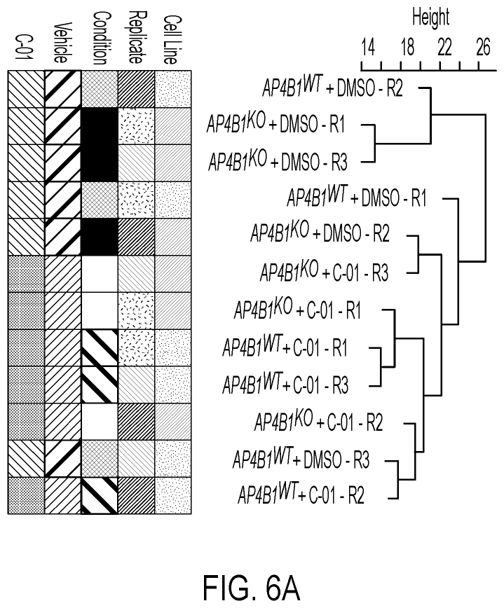

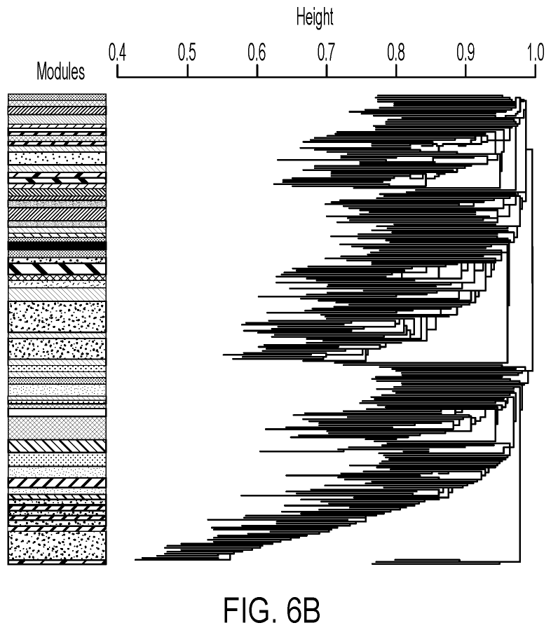

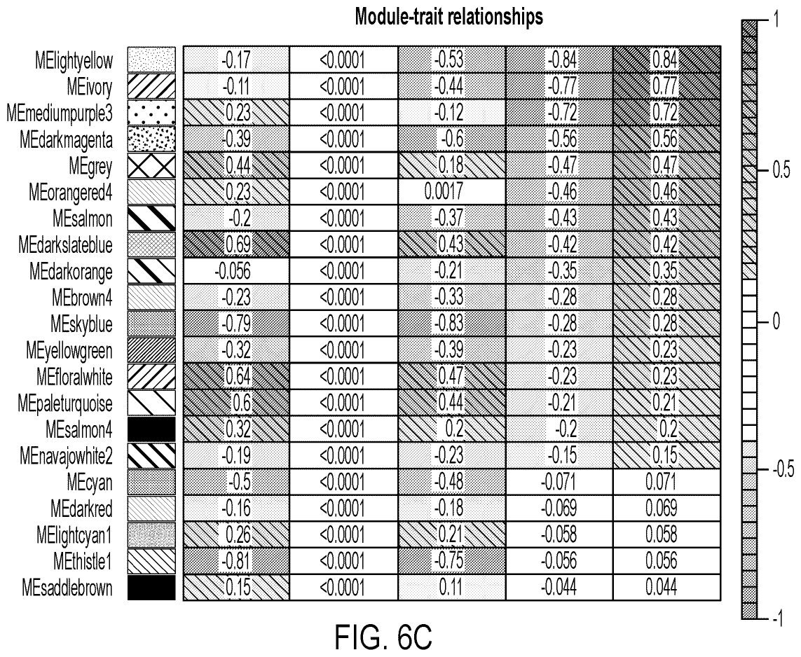

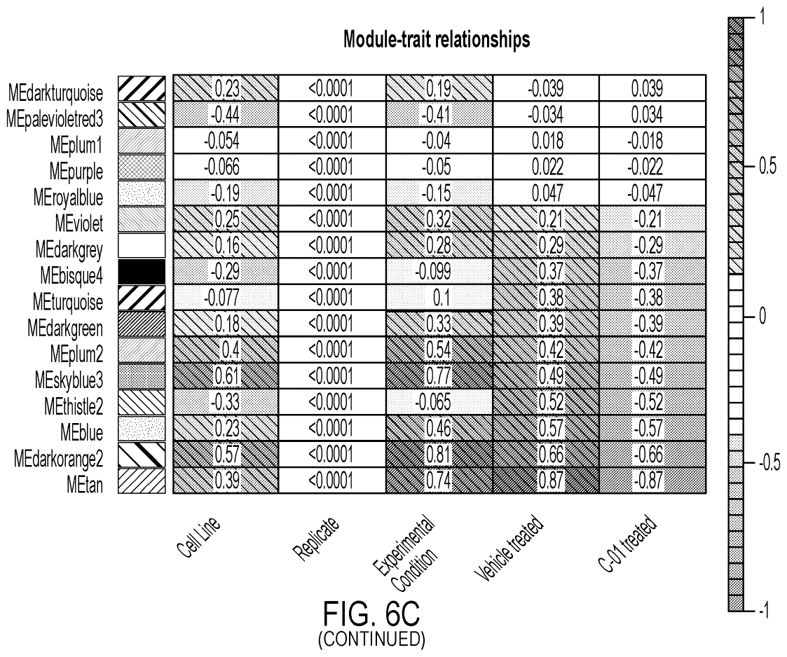

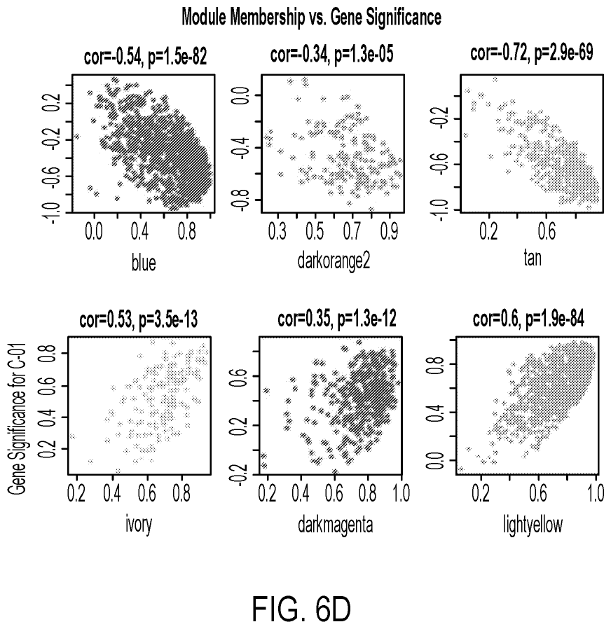

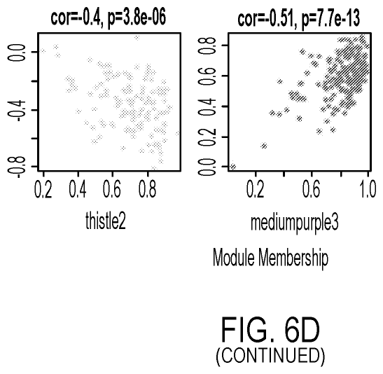

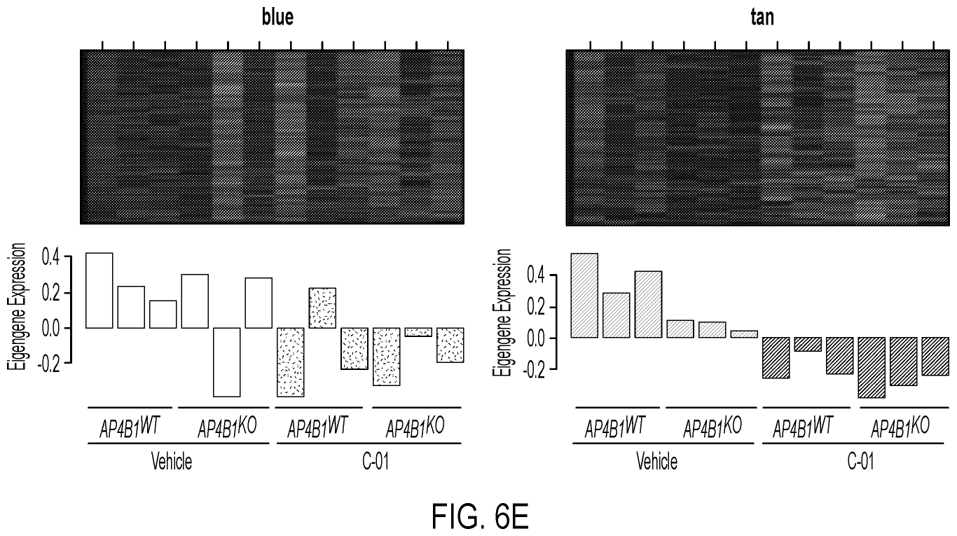

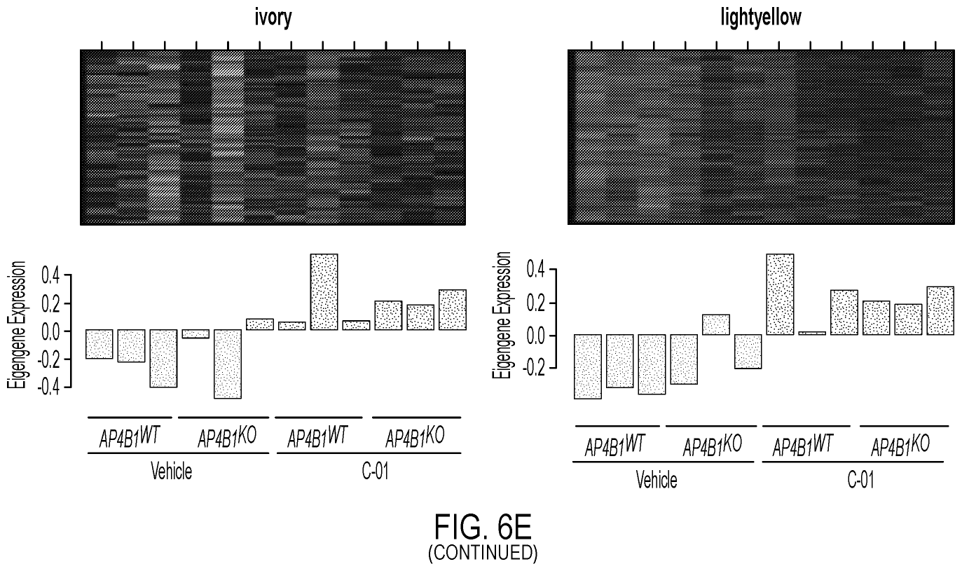

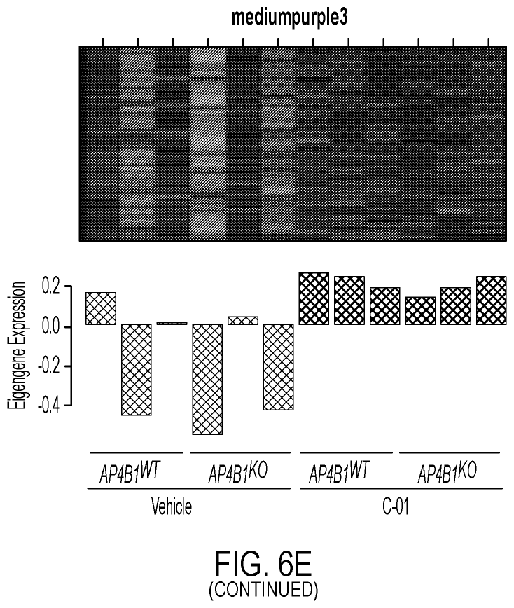

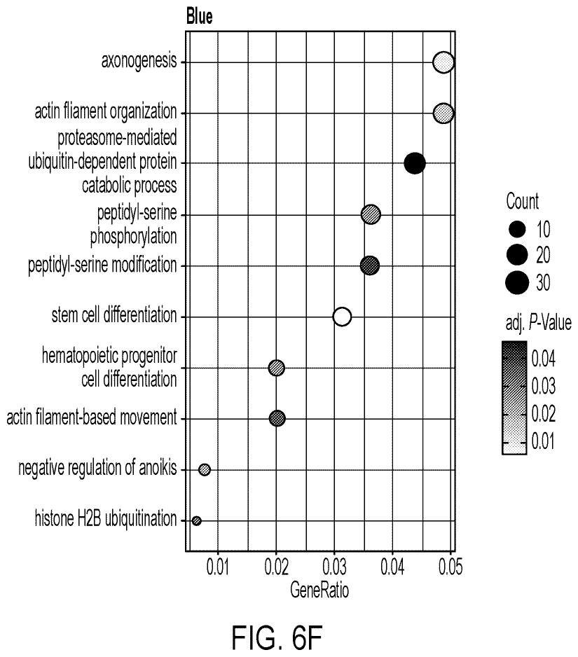

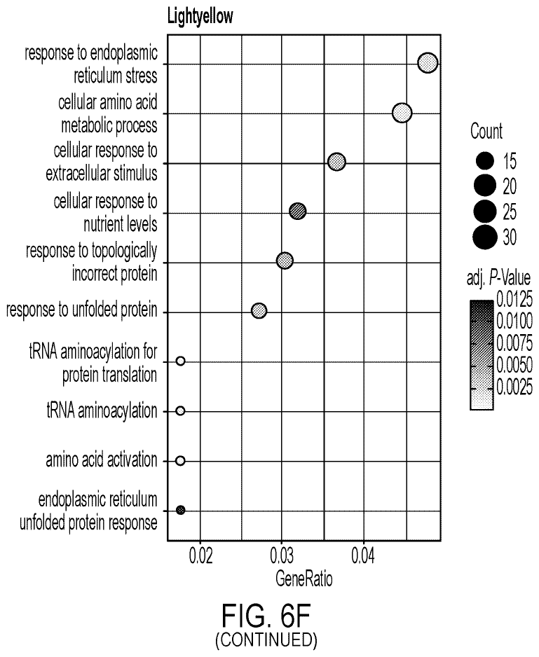

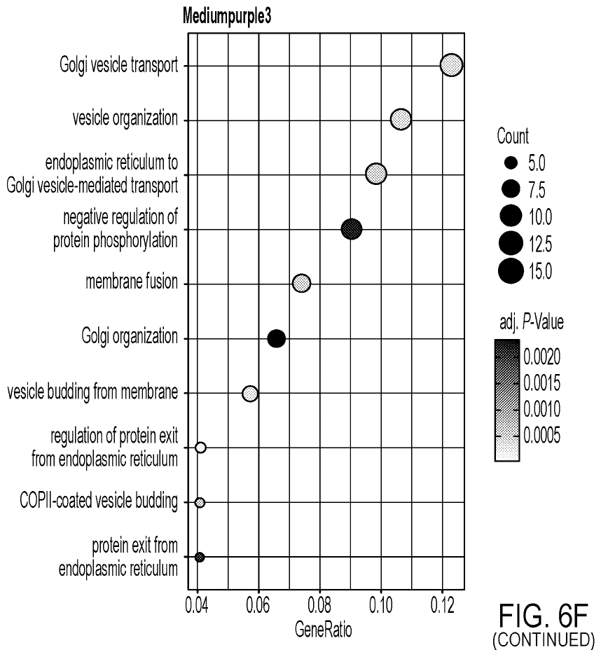

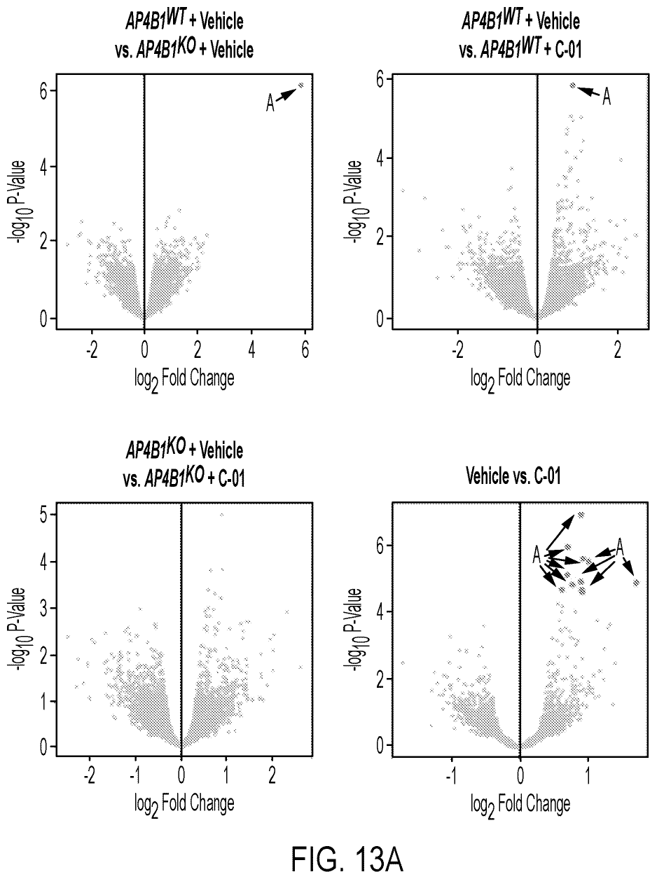

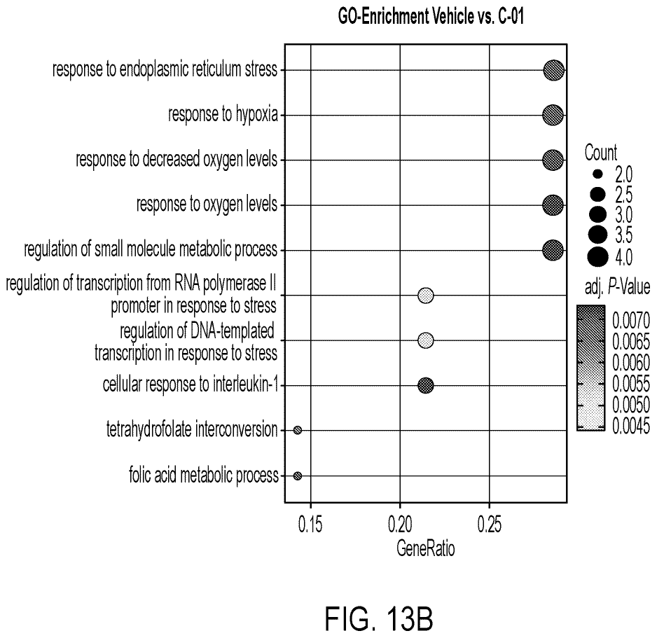

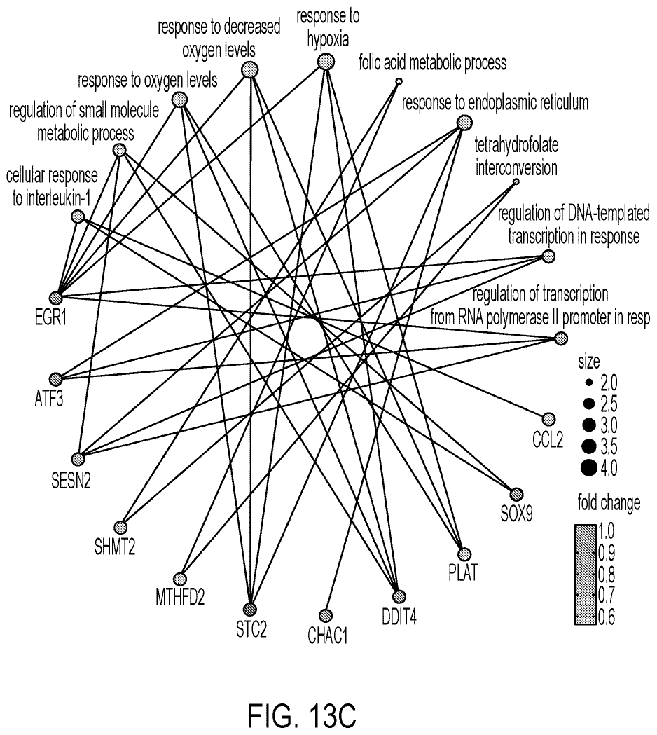

First, bulk RNA sequencing was conducted in differentiated AP4B1^{WT} and AP4B1KO SH-SY5Y cells treated for 72 hours with either vehicle or compound C-01 (5 μM). Analysis of differential gene expression identified very few significant transcriptional changes in response to C-01 treatment, suggesting that this compound does not elicit major alterations in gene expression or induce many off-target effects. (FIG. 13). Since changes in gene expression caused by short-duration small molecule treatments might be too small to reach predefined cut-offs for standard differential expression analyses, and because compounds might affect groups of genes in shared pathways rather that modifying single target genes, an unbiased and unsupervised network approach was adopted to identify groups of co-expressed genes. Hierarchical clustering of samples showed that treatment with C-01, regardless of cell line, was the main differentiator in the dataset (FIG. 6A). To identify the gene networks responsible for these changes, weighted gene co-expression network analysis (WGCNA) was used to group the 18,506 expressed genes into 36 co-expression modules (FIG. 6B). Gene expression profiles within each module were summarized using the “module eigengene” (ME), defined as the first principal component (PC) of a module. Within each module, the association of MEs with measured traits were examined by correlation analysis (FIG. 6C). Eight modules that showed an absolute correlation coefficient >0.5 were selected for further evaluation. For these selected modules, ME based connectivity was determined for every gene by calculating the absolute value of the Pearson correlation between the expression of the gene and the respective ME, producing a quantitative measure of module membership (MM). Similarly, the correlation of individual genes with C-01 treatment was computed, defining gene significance (GS) for C-01. Using the GS and MM, an intramodular analysis was performed, allowing identification of genes that have high significance with treatment as well as high connectivity to their modules (FIG. 6D). Five modules were significantly related to C-01 treatment, defined as showing an absolute correlation coefficient between MM and GS>0.5 (FIG. 6E). To summarize the biological information contained in these modules of interest, gene ontology (GO) analysis was performed, which demonstrated enrichment in biological pathways in three out of the five assessed modules (FIG. 6F). The upper left module (entitled “blue”) showed down-regulation of pathways involved in axonogenesis, actin filament organization and proteasome-mediated pathways. The upper right module (entitled “light yellow”) contained genes involved in ER stress response, amino acid metabolism and transcription. Finally, the lower module (entitled “mediumpurple3”) depicted upregulation of genes involved in vesicular transport, particularly involving TGN and ER-associated transport, as well as membrane and vesicle dynamics. This last module showed the highest gene ratios (defined as the percentage of total differentially expressed genes in the given GO term) and lowest P-values of all differentially regulated pathways across all modules, suggesting the upregulation of alternative vesicle mediated transport mechanisms by compound C-01 (FIG. 6F).

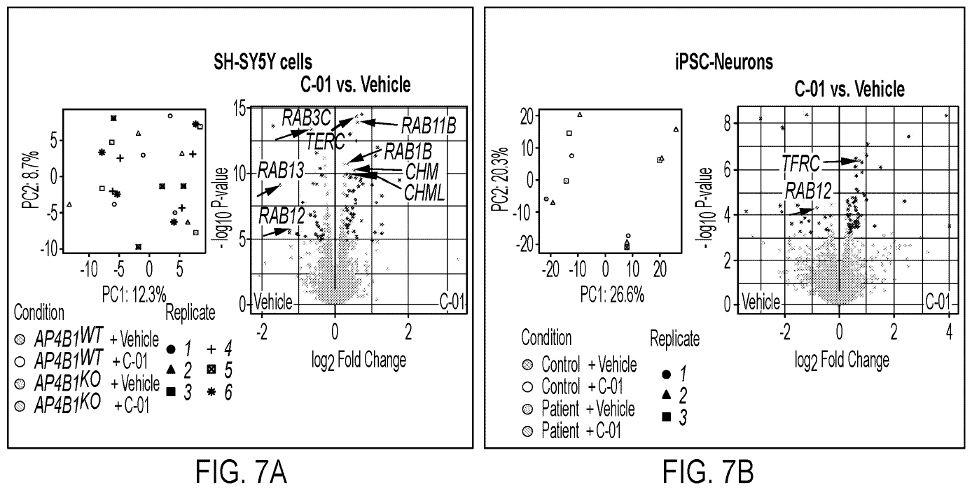

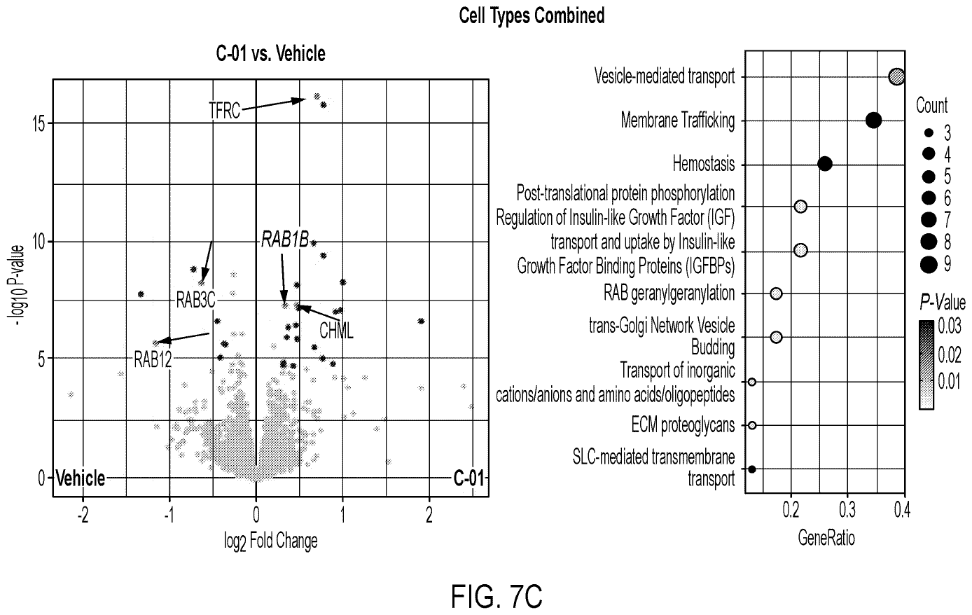

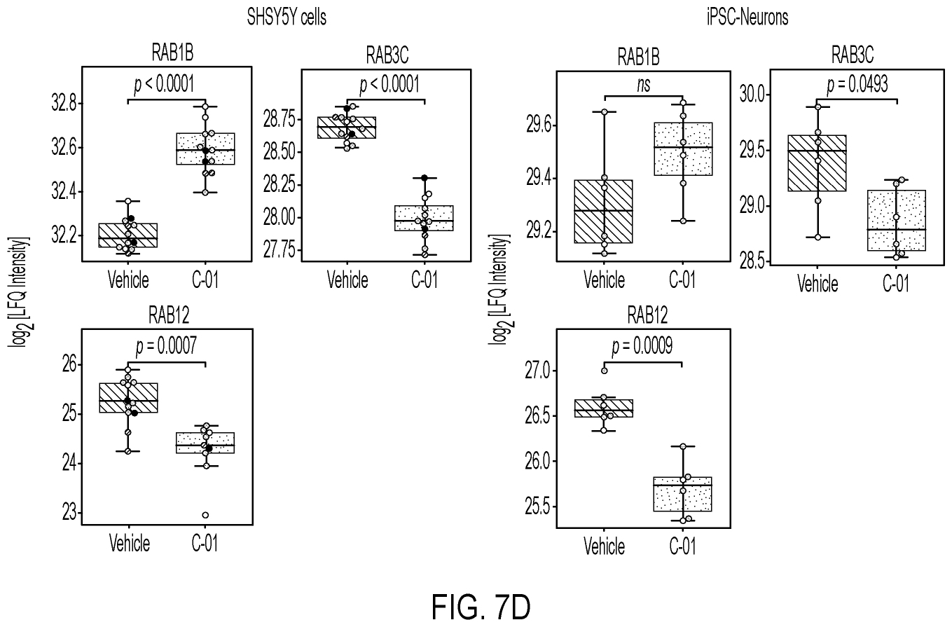

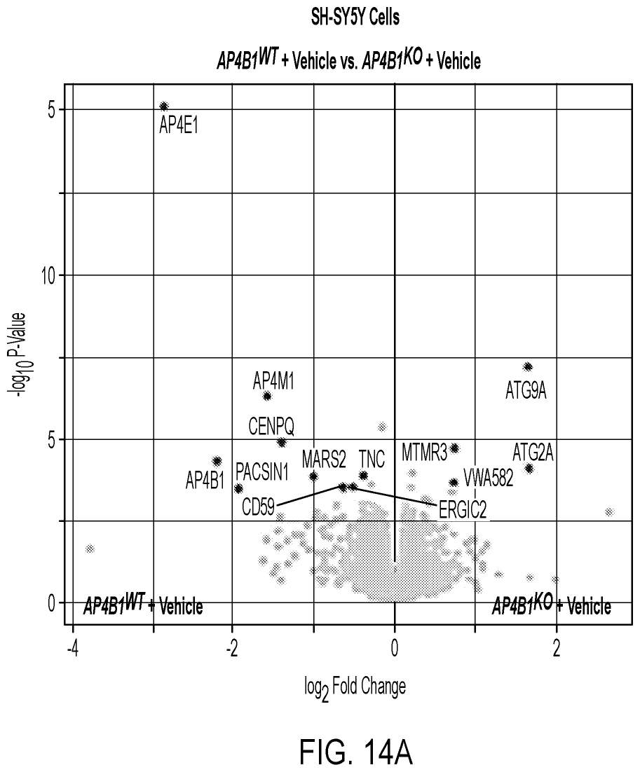

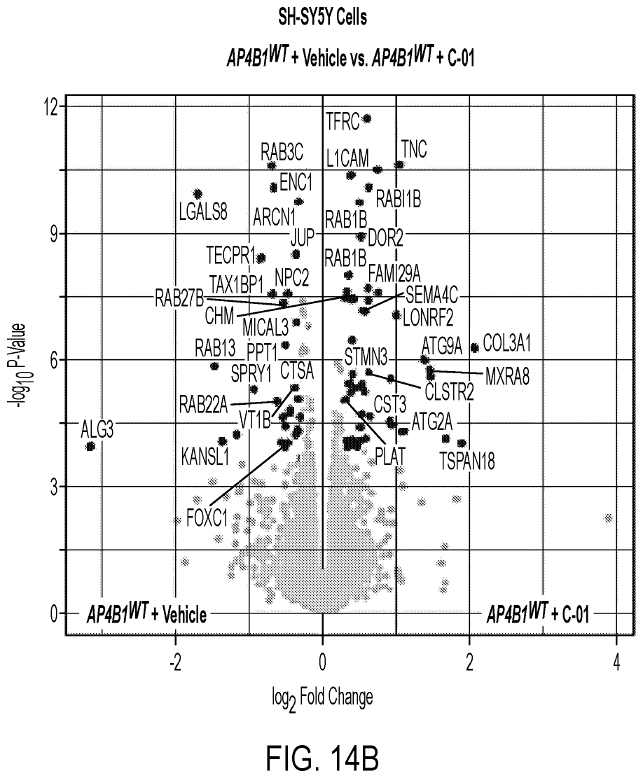

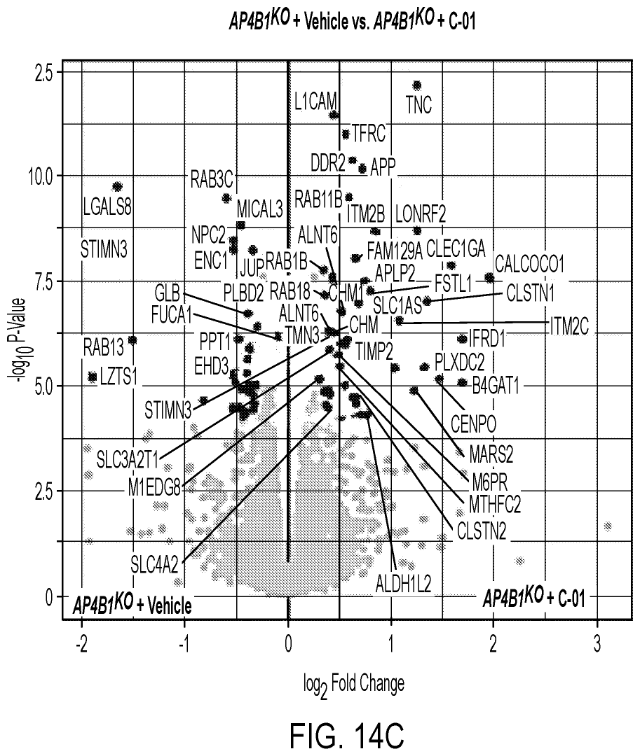

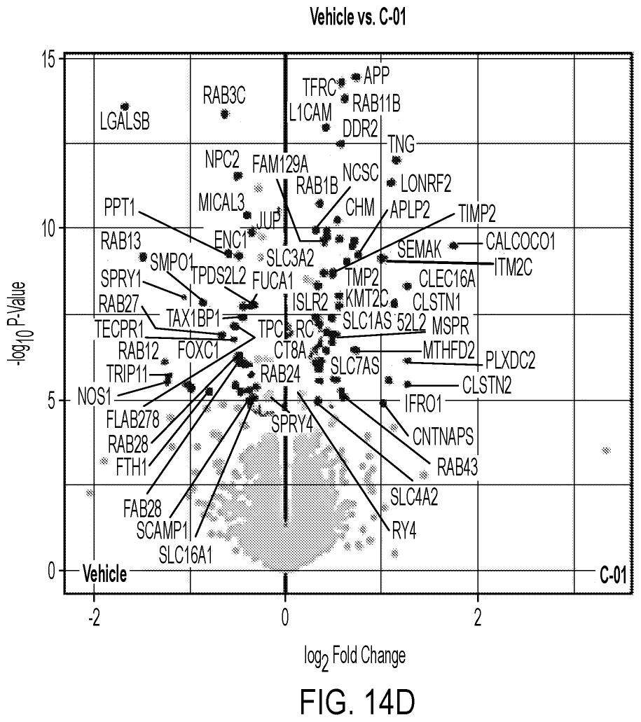

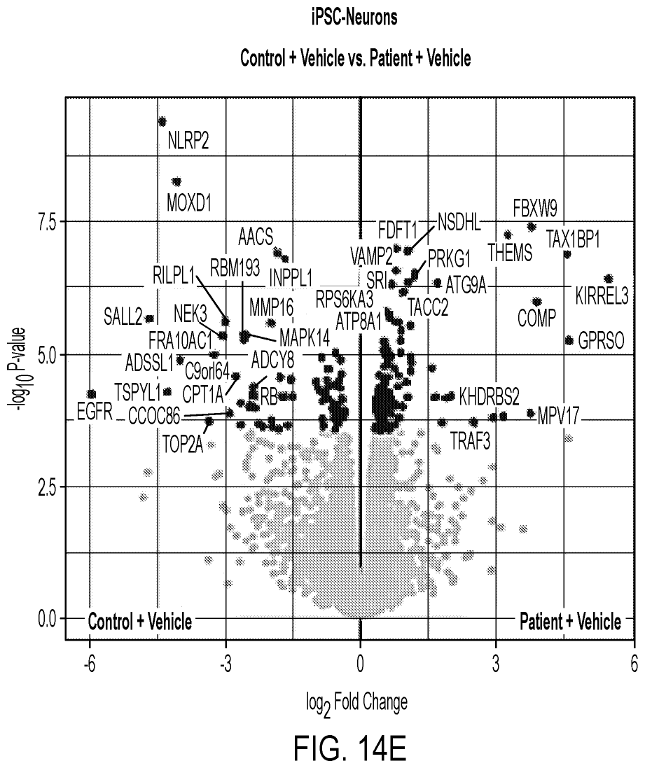

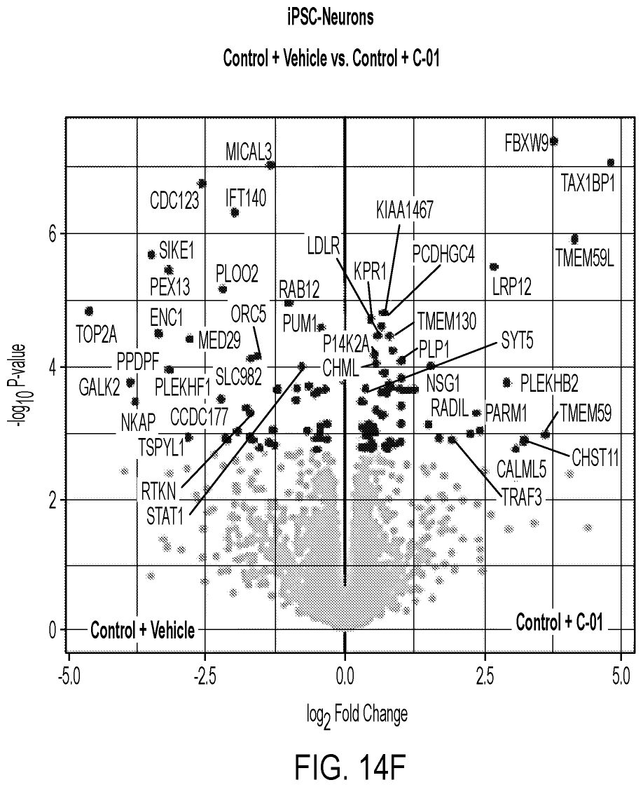

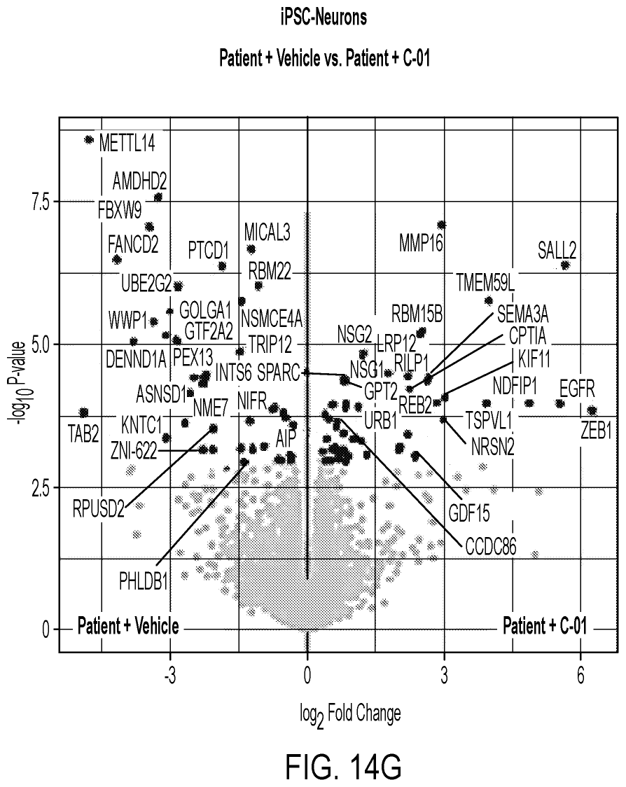

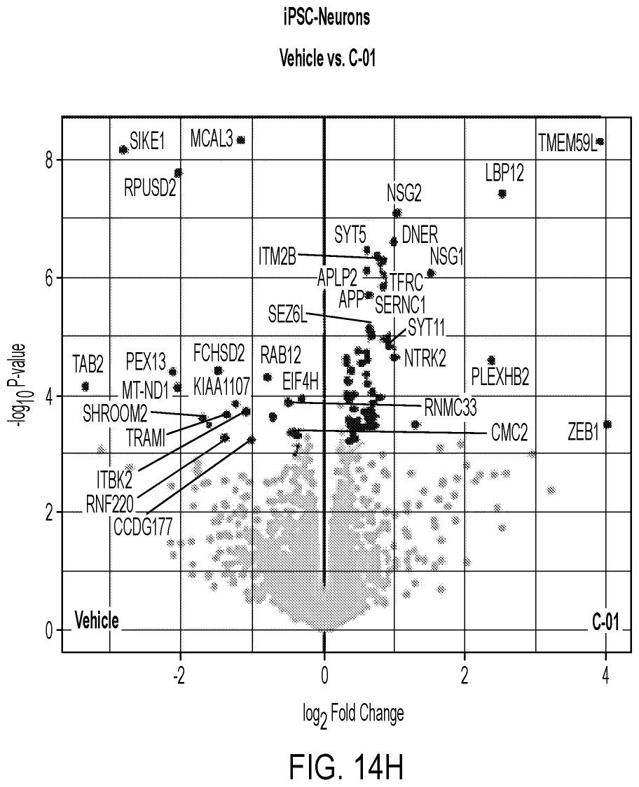

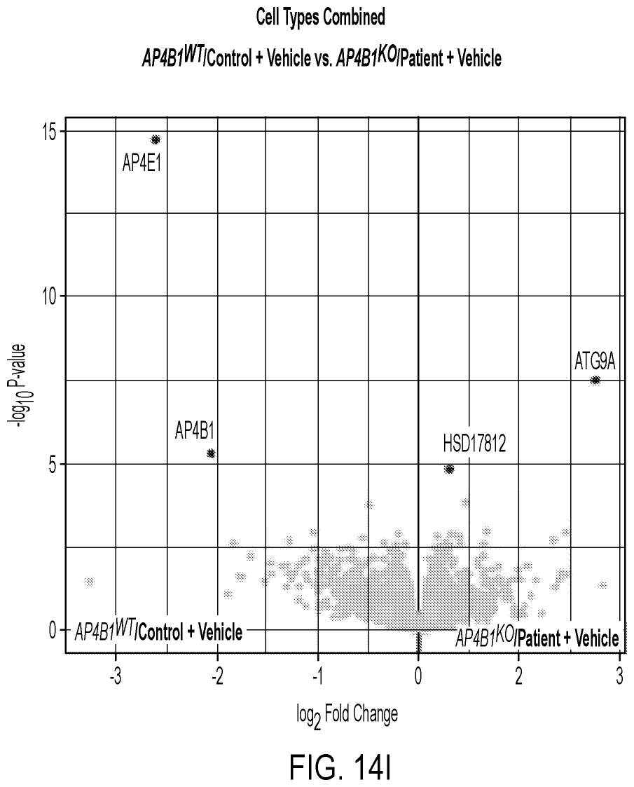

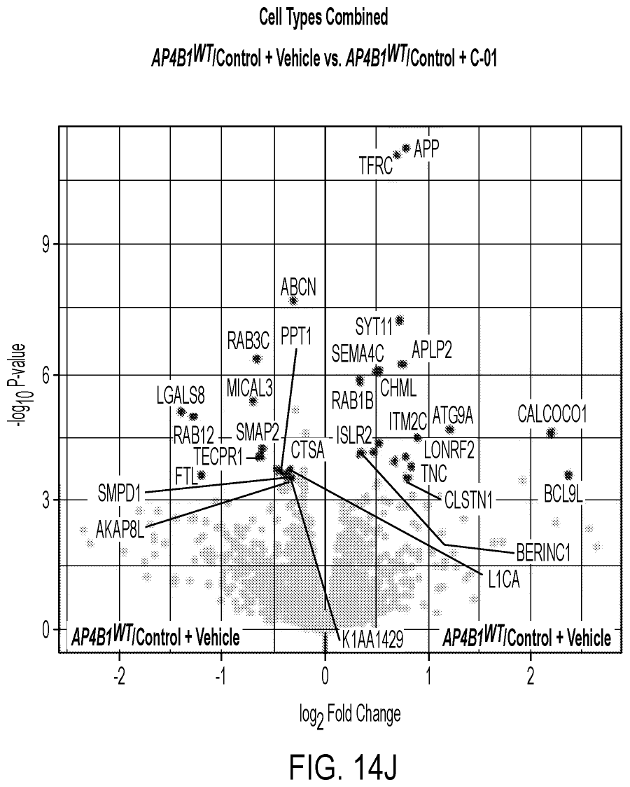

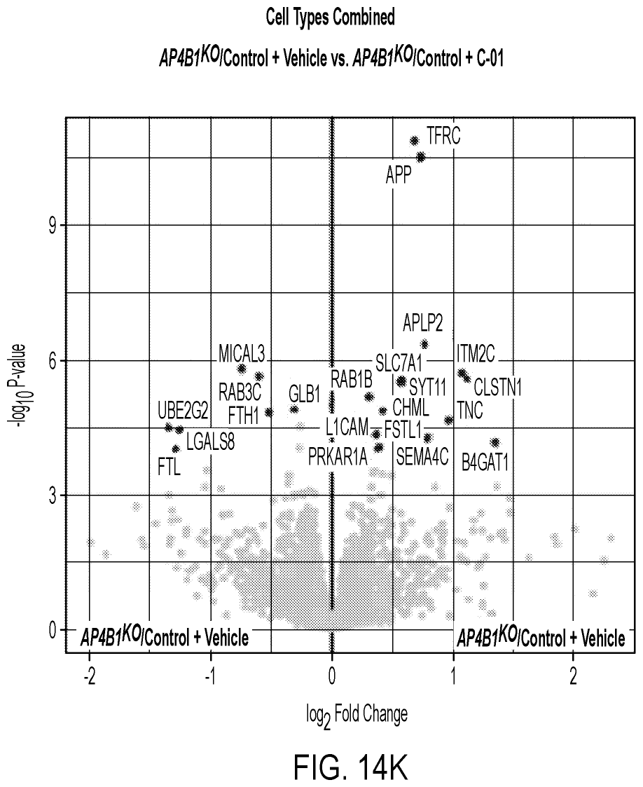

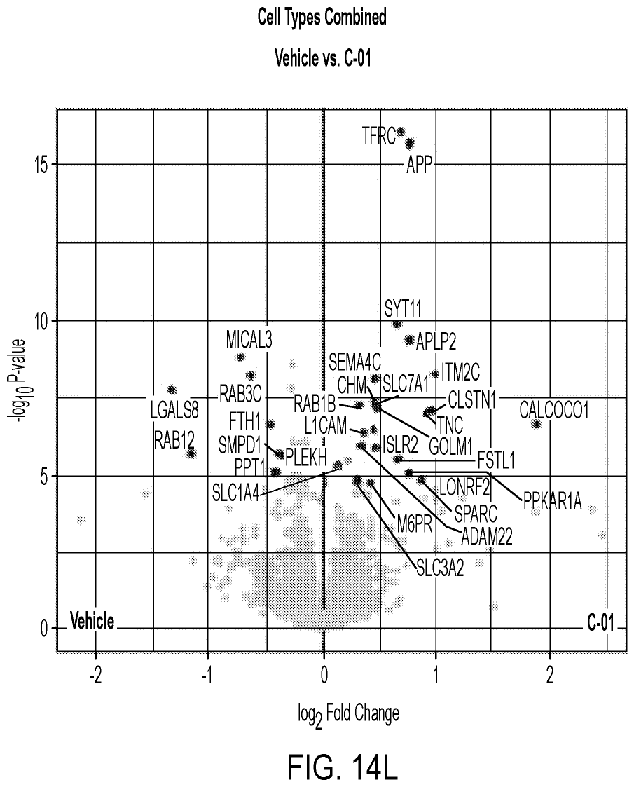

To assess whether similar themes would emerge on the protein level, unbiased quantitative proteomics was next used in both differentiated SH-SY5Y cells (AP4B1^{KO} and AP4B1^{WT}) and iPSC-derived neurons (patient with AP4B1-associated SPG47 and controls) treated for 72 hours with either vehicle or compound C-01 (5 μM). After quality filtering, 8141 unique proteins in SH-SY5Y cells and 7386 unique proteins in iPSC-derived neurons were quantified. Differential enrichment analyses for both cell lines are shown in FIGS. 7A-7B. Baseline quantification of differentially expressed proteins in AP4B1^{KO} SH-SY5Y cells showed downregulation of AP-4 subunits, AP4B1, AP4E1 and AP4M1, and increased ATG9A levels (FIG. 14A). PCA analysis of SH-SY5Y cells demonstrated 4 distinct clusters separated by C-01 treatment (explaining 12.3% of variance) and genotype (explaining 8.7% of variance) (FIG. 7A). Testing of vehicle vs. C-01 treated cells showed broadly similar groups of dysregulated proteins in AP4B1^{WT} and AP4B1^{KO} SH-SY5Y cells (FIGS. 14B-14D), suggesting a conserved mechanism of action independent of genotype, which allowed the pooling of cell lines to increase the power of the analysis (FIG. 7A). Similar observations were made for iPSC-derived neurons (FIG. 7B and FIG. 14E-14H). Here cell lines were a stronger discriminator, likely due to heterogeneity of the positive and negative controls. Again, differentially enriched proteins following C-01 treatment in iPSC-neurons showed a high degree of similarity between patient and control lines (FIGS. 14F-14H), allowing pooling of cell lines (FIG. 7B).

Despite the heterogeneity in the neuronal samples, significant overlap was observed between the differentially enriched proteins in SH-SY5Y cells and iPSC-derived neurons. Datasets were thus integrated for a combined analysis, which detected several proteins that were dysregulated across all cell types and genotypes (FIGS. 14I-14L), providing strong evidence that these changes were related to treatment with C-01 (FIG. 7C). Consistent with the overall changes in gene expression, pathway enrichment analysis using the Reactome database highlighted engagement of intracellular trafficking pathways as a potential mechanism of action for C-01 (FIG. 7C). Specifically, modulation of RAB proteins involved in vesicle transport emerged as a consistent theme across cell types and genotypes, with the strongest evidence for the upregulation of RAB1B and downregulation of RAB3C and RAB12. While C-01 led to a significant change in protein levels of all three RAB protein family members in SH-SY5Y cells, only RAB3C and RAB12 reached significance in neurons (FIG. 7D). This overall pattern of RAB protein modulation was further supported by upregulation of the RAB protein geranylgeranyltransferase components A1 (CHM) in SH-SY5Y cells and A2 (CHML) in both SH-SY5Y cells and neurons, which play a vital role for tethering RAB proteins to intracellular membranes. Additionally, upregulation of transferrin receptor protein 1 (TFRC) was observed FIG. 7C), consistent with prior observations showing that reduction of RAB12 associates with increased protein levels of TFRC³⁸. Collectively, these findings suggest a potential role of RAB proteins in regulating vesicle transport in response to C-01 treatment.

### RAB3C and RAB12 Knockout are Involved in C-01-Mediated Vesicle Trafficking and Autophagy

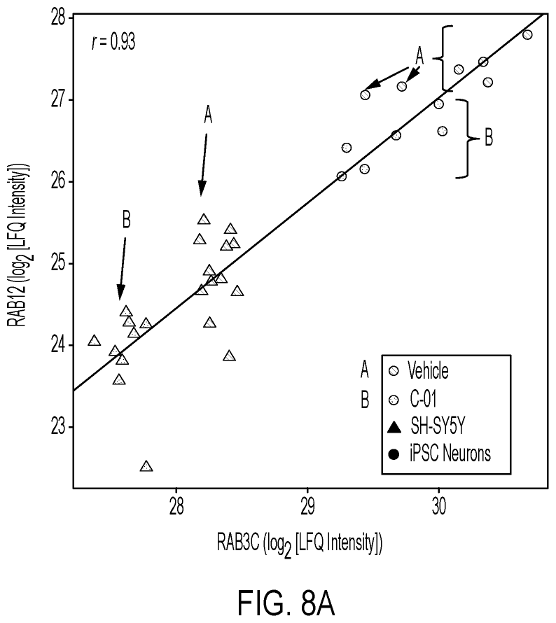

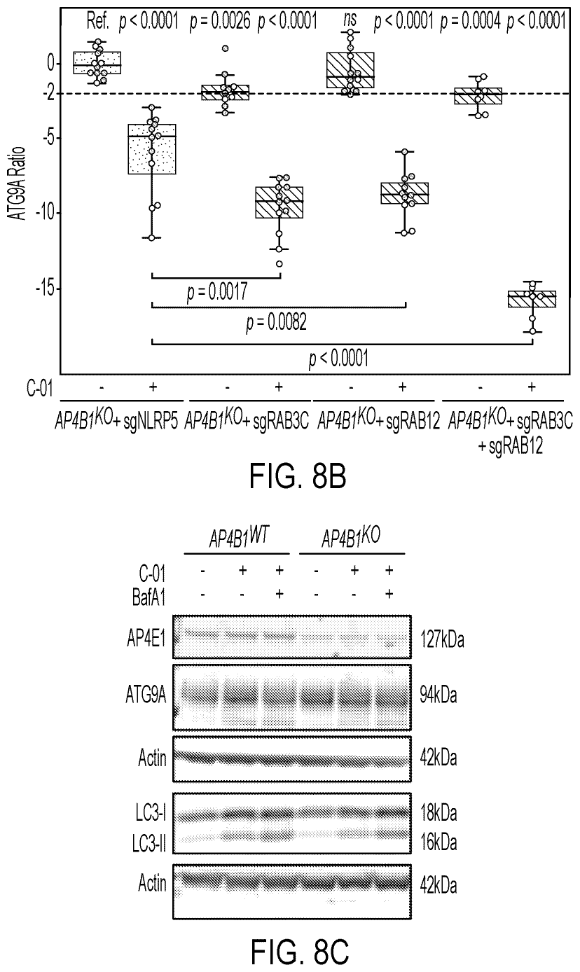

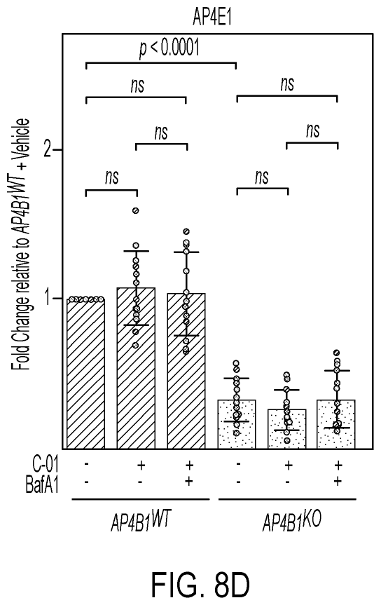

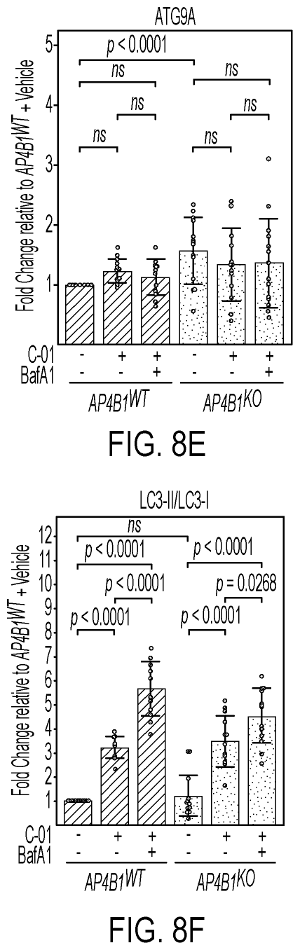

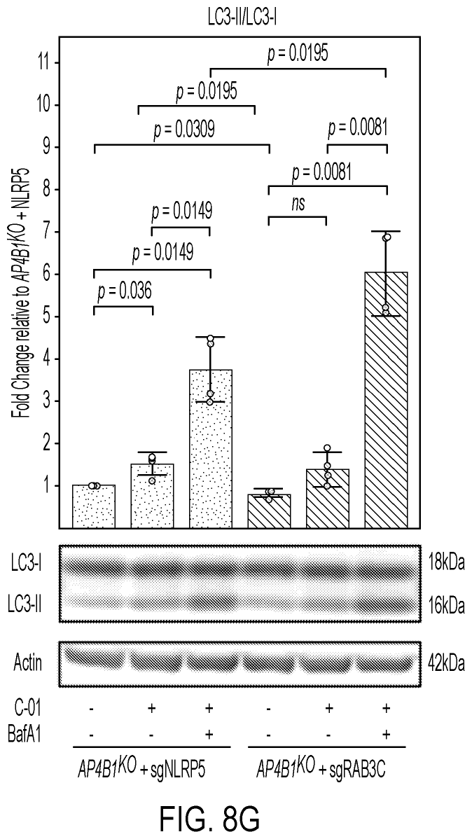

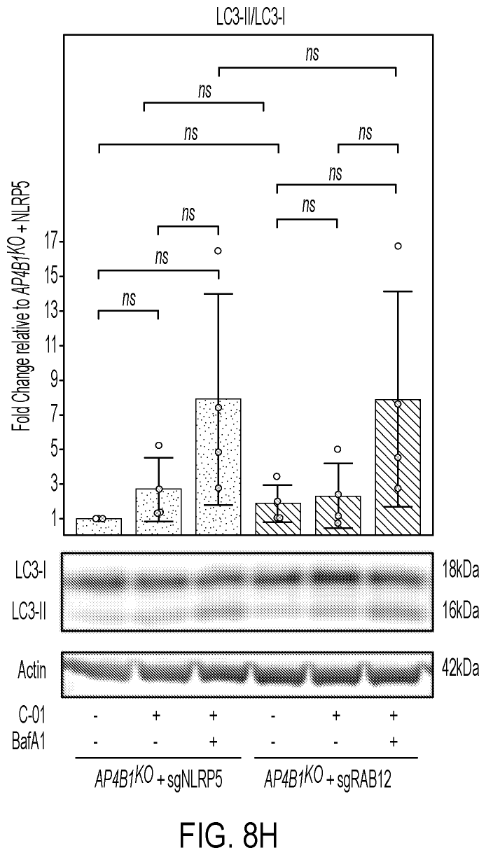

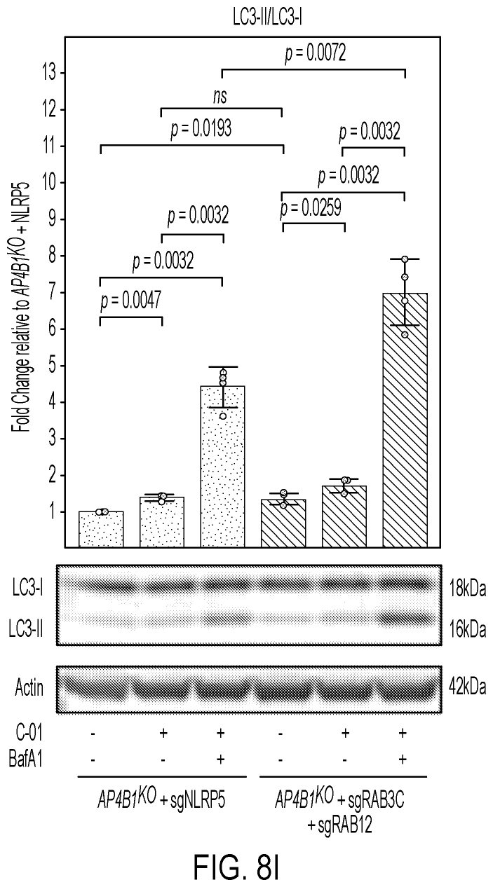

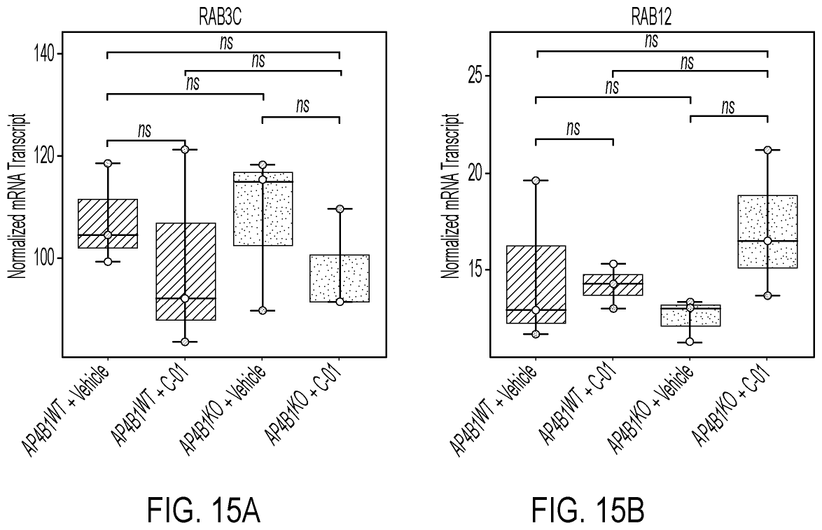

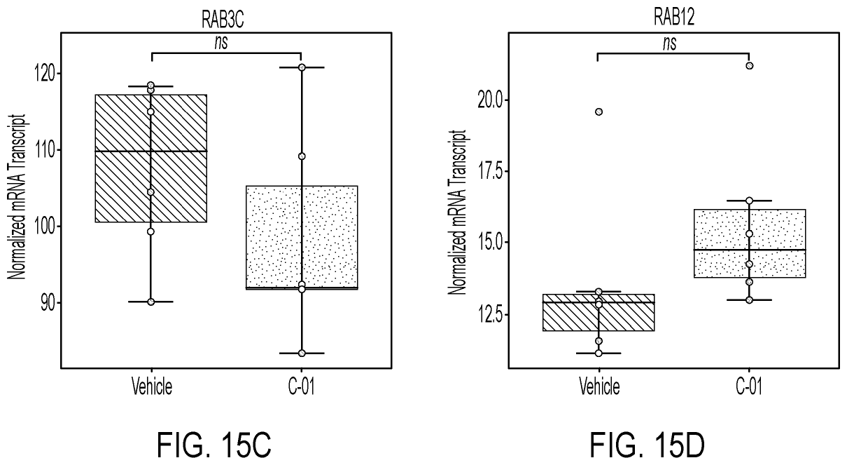

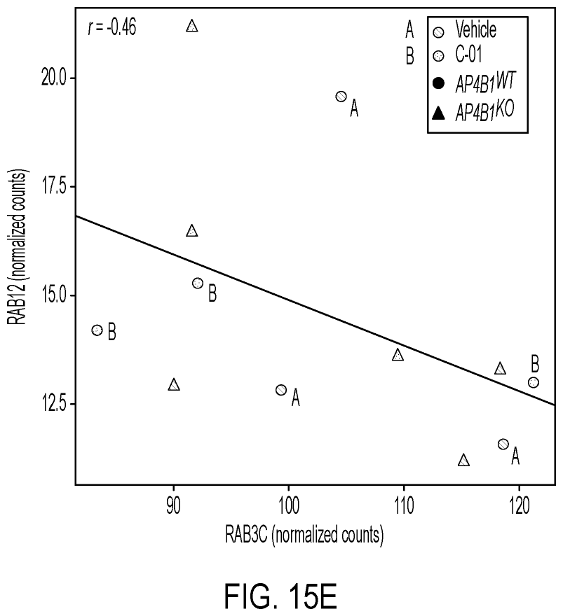

RAB3C and RAB12 exhibited the strongest and most consistent protein expression changes in both differentiated SH-SY5Y cells and iPSC-derived neurons following treatment with C-01 (FIG. 7D) and were therefore selected for further investigation. Correlation analysis revealed a strong correlation (r=0.93) between the LFQ intensities of these two proteins in both cell types and across different genotypes in response to C-01 (FIG. 8A). To assess whether a correlation was also present on the transcriptional level, mRNA levels of RAB3C and RAB12 in response to C-01 treatment were analyzed in AP4B1^{WT} and AP4B1^{KO} SH-SY5Y cells (FIGS. 15A-15E). While no significant differences were detected, there was a trend toward a reduction of RAB3C (FIGS. 15A, FIG. 15C) and elevation of RAB12 (FIG. 15B, FIG. 15D) mRNA levels. Correlation analysis demonstrated a moderate inverse correlation between expression levels of RAB3C and RAB12 (FIG. 15E).

To investigate the potential impact of RAB3C and RAB12 on ATG9A translocation in the AP-4 deficient background, CRISPR/Cas9-mediated knockouts of RAB3C and RAB12 in AP4B1^{KO} SH-SY5Y cells was used (FIG. 8B). Knockout of RAB12 was found to not affect ATG9A translocation, while knockout of RAB3C caused a moderate reduction in the ATG9A ratio (−2 SD). However, the combined knockout of RAB3C and RAB12 in AP4B1^{KO} SH-SY5Y cells did not show any additive effect. The effects of C-01 on ATG9A translocation were significantly enhanced by knockout of either RAB3C or RAB12 alone, and even further augmented with the combined knockout of both genes. These findings suggest that both RAB3C and RAB12 play a role in C-01-mediated ATG9A redistribution.

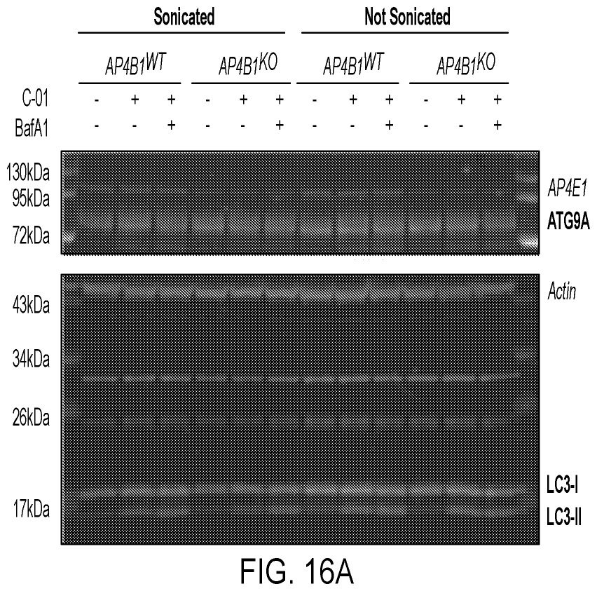

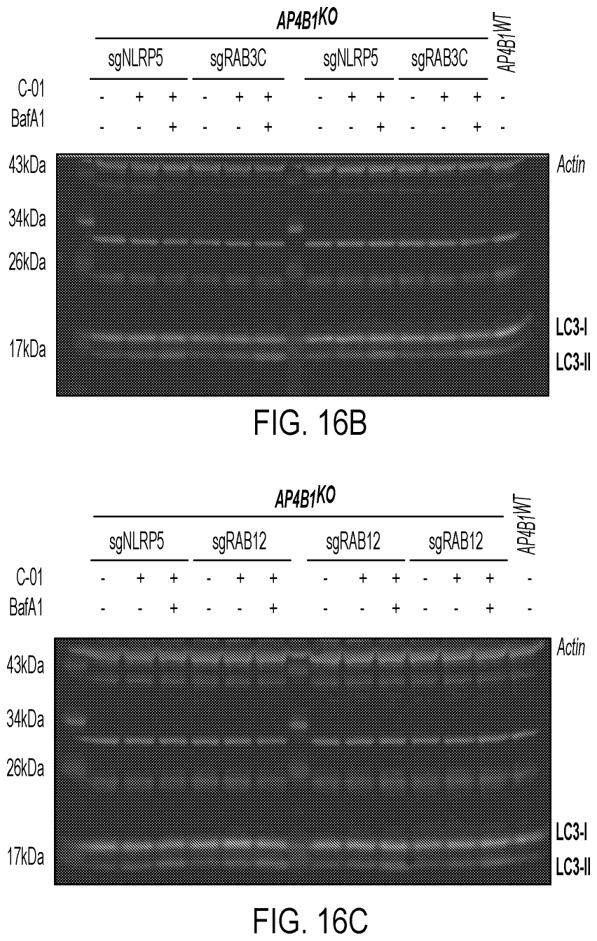

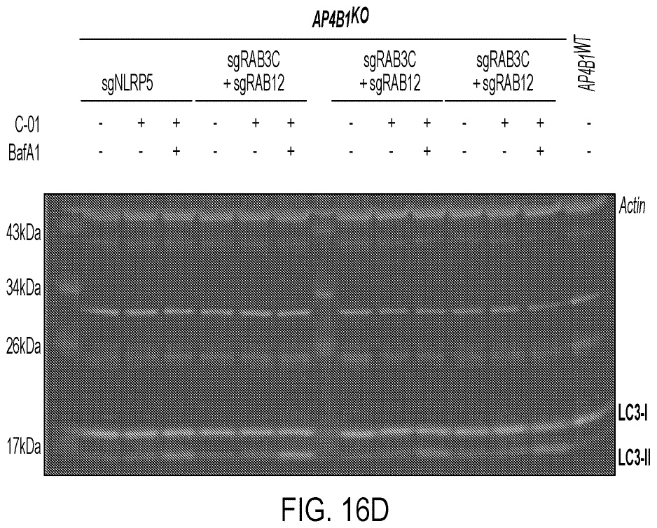

A converging theme of ATG9A translocation and alteration of RAB protein expression is modulation of autophagy. To investigate for changes in autophagic flux, AP4B1^{WT} and AP4B1^{KO} SH-SY5Y cells were treated with C-01 for 72 hours and LC3-I to LC3-II conversion was measured by western blotting (FIGS. 8C-8F and FIG. 16A). Levels of LC3-II were significantly elevated in all cell lines treated with C-01, suggesting modulation of the autophagy pathway. Co-treatment with bafilomycin A1 for 4 hours, which blocks autophagosome-lysosome fusion, led to further LC3-II accumulation, indicating that C-01 increases autophagic flux (FIGS. 8C-8F).

To investigate the potential contribution of RAB3C and RAB12 to the modulation of autophagy, CRISPR/Cas9-mediated knockouts of RAB3C and RAB12 were introduced in AP4B1^{KO} SH-SY5Y cells (FIGS. 8G-8H and FIGS. 16B-16D). Neither RAB3C nor RAB12 knockout alone led to changes in baseline or C-01-enhanced autophagic flux (FIGS. 8G-8H). However, combined knockout of RAB3C and RAB12 significantly increased the conversion of LC3-II to LC3-I (FIG. 8I). Upon treatment with bafilomycin A1, both RAB3C knockout alone and combined knockout of RAB3C and RAB12 further increased C-01-mediated LC3-II to LC3-I conversion (FIGS. 8G-8I). These findings suggest the possibility that RAB3C and RAB12 modulate C-01-mediated ATG9A trafficking and autophagy induction, at least partially explaining some of the observed effects of C-01.

### Discussion

Identification of therapeutic targets for rare neurological diseases represents a major scientific and public health challenge. The increasing number of rare genetic diseases, the rising rate of diagnoses, and the significant burden for patients, caregivers and health care systems highlight the urgent need for translational research that moves beyond gene discovery to the identification of disease mechanisms and therapies.

Unbiased high-content small molecule screens are a platform for both drug-repurposing approaches and a starting point for the rationale development of new compounds. Disease-relevant, ‘screenable’ phenotypes across cellular models, including patient-derived cells, provide an entry point into developing automated, high-content screening and analysis platforms.

In the present disclosure, the first high-throughput cell-based phenotypic screening platform was developed for a prototypical form of childhood-onset HSP caused by defective protein trafficking. The platform allows the user to determine the subcellular localization of the AP-4 cargo protein ATG9A in several cellular models of AP-4-deficiency based on ATG9A mislocalization being a key mechanism in the pathogenesis of AP-4-HSP.

ATG9A is the only conserved transmembrane autophagy-related protein and in mammalian cells cycles between the TGN and ATG9A vesicles, which associate with endosomes and autophagosome formation sites. ATG9A has 4 transmembrane domains and forms homotrimers that have lipid scramblase activity, postulated to equilibrate lipids in the double-membrane layer of nascent autophagosomes. Basal levels of autophagy are essential for neuronal survival, and neuron-specific ablation of the autophagy pathway leads to axonal degeneration and cell death. In neurons, autophagosomes form in the distal axon and are subject to active transport, thus efficient vesicular trafficking and spatial distribution of ATG9A are essential for axonal function as demonstrated in CNS-specific Atg9a knockout mice.

Having established a robust and dynamic assay that reliably measures intracellular ATG9A distribution, a large library of 28,864 novel small molecules was systematically screened for their ability to restore ATG9A trafficking from the TGN to the cytoplasm. Following this primary screen, a counter-screen and a series of orthogonal experiments using unbiased multiparametric analyses, identified the novel small molecule C-01 that can restore the intracellular distribution of ATG9A and a second transmembrane protein and AP-4 cargo, DAGLB, in neuronal models of AP-4 deficiency, including iPSC-derived neurons from two patients with AP-4-HSP.

Since the molecular targets of C-01 are unknown, a target deconvolution strategy was employed using transcriptomics and proteomic profiling to define the cellular pathways impacted by this novel small molecule. This approach identified two central themes: 1) modulation of Golgi dynamics and vesicular trafficking and 2) engagement of autophagy. At the core of the putative pathways affected by C-01, the Rab proteins RAB1B, RAB3C and RAB12 were identified, as well as the interacting Rab geranyl transferase subunits CHM and CHML. RAB3C and RAB12 showed the strongest and most consistent association with C-01 treatment in both SH-SY5Y cells and iPSC-derived neurons, and the analyses suggested that these two proteins are involved in C-01-mediated redistribution of ATG9A vesicles and increase of autophagic flux.

Rab proteins comprise a large family of small guanosine triphosphate (GTP) binding proteins that act as key regulators of intracellular membrane trafficking in eukaryotic cells, at every stage including cytoplasmic cargo sorting, vesicle budding, docking, fusion and membrane organization. Rab GTPases function both as soluble and specifically localized, integral-membrane proteins, the latter being mediated by prenylation. Among the roughly 70 known Rab proteins, more than 20 are primarily associated with the TGN, where they regulate Golgi organization, coordinate vesicle trafficking and interact with various steps of the autophagy pathway.

Following treatment with C-01, the RAB protein family members RAB3C and RAB12 were consistently downregulated in both SH-SY5Y cells and iPSC-derived neurons, and knockout experiments of these two proteins suggested potentiation of C-01-mediated ATG9A translocation and autophagic flux. RAB3C, which is part of the RAB3 superfamily, is primarily expressed in brain and endocrine tissues, where it localizes to the Golgi and synaptic vesicles and is involved in exocytosis and modulation of neurotransmitter release. On the other hand, RAB12 is known to regulate endosomal trafficking and lysosomal degradation and has been identified as a modulator of autophagy through negative regulation of mechanistic target of rapamycin complex 1 (mTORC1). RAB12 is mainly localized to recycling endosomes where its known cargo is the transferrin receptor (TfR). Knockdown of RAB12 in mouse embryonic fibroblasts increased TfR protein levels, while overexpression led to its reduction. In line with this, treatment with C-01 was found to reduce RAB12 protein levels while robustly elevating transferrin receptor protein 1 (TFRC). While no interaction between RAB3C and RAB12 has been reported in the literature, the data suggest that both proteins might be involved in C-01-mediated modulation of vesicle trafficking and autophagic flux.

This approach illustrates the development of a small molecule screening platform for a rare genetic disease, leveraging robust cellular phenotypes. This approach may create a paradigm for other rare and more common disorders of protein trafficking. The increase of autophagic flux through C-01 offers the intriguing possibility that this compound could be considered for the treatment of other autophagy-associated diseases.

### Materials and Methods

Clinical data from patients with AP-4-HSP This study was approved by the Institutional Review Board at Boston Children's Hospital (IRB-P00033016 and IRB-P00016119). Two patients with AP-4-HSP and their clinically-unaffected, sex-matched parents were enrolled International Registry and Natural History Study for Early-Onset Hereditary Spastic Paraplegia (ClinicalTrials.gov Identifier: NCT04712812). Both patients had a clinical and molecular diagnosis of AP-4-HSP and presented with core clinical and imaging features 8. Patient 1 was diagnosed with AP4B1-associated SPG47 and carries the following compound heterozygous variants: NM_001253852.3, c.1160_1161del (p.Thr387ArgfsTer30)/c.1345A>T (p.Arg449Ter). The sex-matched parent carries the heterozygous c.1160_1161del; p.Thr387Argfs*30 variant. Patient 2 was diagnosed with AP4M1-associated SPG50 and carries the following compound-heterozygous variants: NM_004722.4, c.916C>T (p.Arg306Ter)/c.694dupG (p.Glu232GlyfsTer21). The sex-matched parent carries the heterozygous c.694dupG (p.Glu232GlyfsTer21) variant.

Antibodies and reagents. The following reagents were used: Bovine serum albumin (AmericanBIO, Cat #9048-46-8), saponin (Sigma, #47036-50G-F), normal goat serum (Sigma-Aldrich, Cat #G9023-10ML), Dulbecco's phosphate-buffered saline (DPBS) (Thermo Fisher Scientific, Cat #14190-250), trypsin (Thermo Fisher Scientific, Cat #25200056), 4% paraformaldehyde (4%) (Boston BioProducts, Cat #BM-155), dimethyl-sulfoxide (DMSO) (American Bioanalytical, Cat #AB03091-00100), Bafilomycin A1 (Enzo Life Sciences, Cat #BML-CM110-0100), Molecular Probes Hoechst 33258 (Thermo Fisher Scientific, Cat #H3569) and ALEXA FLUOR™ 647-labelled phalloidin (Thermo Fisher Scientific, Cat #A22287). The following primary antibodies were used: Anti-AP4E1 at 1:500 (BD Bioscience, Cat #612019), anti-ATG9A at 1:500-1000 (Abcam, Cat #ab108338), anti-DAGLB at 1:500 (Abcam, Cat #191159), anti-TGN46 at 1:800 (Bio-Rad, Cat #AHP500G), anti-Golgi 97 1:500 (Abcam, Cat #169287), anti-beta-Tubulin III 1:1000 (Synaptic Systems, Cat #302304), anti-beta-Actin 1:10,000 (Sigma, Cat #A1978-100UL), anti-LC3B 1:1000 (Novus, Cat #100-2220). Fluorescently labelled secondary antibodies for immunocytochemistry were used at 1:2000 (Thermo Fisher Scientific, Cat #A11008, A11016, A21245), for western blotting at 1:5000 (LI-COR Biosciences, Cat #926-68022, 926-68023, 926-32212, 926-32213).

Small molecule library. A diversity small molecule library containing 28,864 compounds was provided by Astellas Pharma Inc. Compounds were arrayed in 384-well microplates at a final concentration of 10 mM (1000-fold the screening concentration) in DMSO. Assay plates were stored at −80° C. and thawed 30 minutes prior to cell plating. Active compounds from the primary screen were re-screened in a secondary screen, using eleven-point concentrations (range: 0.04 μM, 0.08 μM, 0.16 μM, 0.31 μM, 0.63 μM, 1.25 μM, 2.5 μM, 5 μM, 10 μM, 20 μM, 40 μM) in two biological replicates.

Fibroblast cell culture. Fibroblast lines were established from routine skin punch biopsies in both patients and their respective sex-matched heterozygous parents. Primary human skin fibroblasts were cultured and maintained. Briefly, cells were maintained in DMEM high glucose (Gibco, #11960044) supplemented with 20% FBS (Gibco, #10082147), penicillin 100U/ml and streptomycin 100 μg/ml (Gibco, #15140122). Cells were kept in culture for up to 8 passages and routinely tested for the presence of Mycoplasma contamination. For high-throughput imaging, fibroblasts were seeded onto 384-well plates (Greiner Bio-One, #781090) at a density of 2×10³ per well using the Multidrop Combi Reagent Dispenser (Thermo Fisher Scientific, #11388-558). Media changes were done every 2-3 days and drugs administered 24 hours before fixation.

SH-SY5Y cell culture. AP4B1 wild type (AP4B1^{WT} and AP4B1 knockout (AP4B1KO)) SH-SY5Y cells were generated previously. Undifferentiated SH-SY5Y cells were maintained in DMEM/F12 (Gibco, Cat #11320033) supplemented with 10% heat-inactivated fetal bovine serum (Gibco, Cat #10438026), 100U/ml penicillin and 100 g/ml streptomycin at 37° C. under 5% CO₂. SH-SY5Y cells were passaged every 2-3 days and differentiated into a neuron-like state using a 5-day differentiation protocol with all-trans-retinoic acid (MedChemExpress, #HY-14649). For assessment of ATG9A translocation, differentiated SH-SY5Y cells were plated in 96-well plates (Greiner Bio-One, Cat #655090), at a density of 10,000 cells per well. Media changes were done every 2-3 days and drugs administered 24-72 hours before fixation.

Generation of iPSC lines and neuronal differentiation. Fibroblasts were reprogrammed to iPSCs using non-integrating Sendai virus. Quality control experiments including karyotyping, embryoid body formation, pluripotency marker expression, STR profiling and Sanger sequencing for AP-4 mutation confirmation were reported previously. Cortical neurons were differentiated according to protocols published by Zhang et al. (Zhang Y, et al. Neuron 78, 785-798 (2013)). For assessment of ATG9A translocation, neurons were plated in 96-well plates at a density of 40,000 cells per well. Media changes were done every 2-3 days and drugs administered 24-72 hours before fixation.

Immunocytochemistry. The immunocytochemistry protocol was optimized for high-throughput staining by using automated pipettes and reagent dispensers (Thermo Fisher Scientific MULTIDROP™ Combi Reagent Dispenser, Integra VIAFLO 96/384 liquid handler, Integra VOYAGER pipette). Fibroblasts and SH-SY5Y cells were fixed using 3% and 4% PFA, respectively, permeabilized with 0.1% saponin in PBS and blocked in 1% BSA/0.01% saponin (blocking solution) in PBS. iPSC-derived neurons were fixed in 4% PFA, and permeabilized and blocked using 0.1% TRITON™ X-100/2% BSA/0.05% NGS in PBS. Primary antibody (diluted in blocking solution) was added for 1 hour (fibroblasts and SH-SY5Y cells) at room temperature or overnight (iPSC neurons) at 4° C. Plates were gently washed three times in blocking solution (fibroblasts and SH-SY5Y cells) or in PBS (iPSC neurons), followed by addition of fluorochrome-conjugated secondary antibodies, Hoechst 33258 and phalloidin for 30 minutes (fibroblasts) or Hoechst 33258 for 60 minutes (SH-SY5Y cells and iPSC neurons) at room temperature. Plates were then gently washed three times with PBS and covered from light.

High-content imaging and automated image analysis. High-throughput confocal imaging was performed on an ImageXpress Micro Confocal Screening System (Molecular Devices) using an experimental pipeline. For experiments in fibroblasts, images were acquired using a 20×S Plan Fluor objective (NA 0.45 μM, WD 8.2-6.9 mm). Per well, 4 fields were acquired in a 2×2 format (384-well plates). For experiments in SH-SY5Y cells and iPSC neurons, up to 36 fields were acquired in a 6×6 format (96-well plate) using a 40×S Plan Fluor objective ((NA 0.60 μm, WB 3.6-2.8 mm). The image analysis was performed using a customized image analysis pipeline in MetaXpress (Molecular Devices): Briefly, cells were identified based on the presence of DAPI signal inside a phalloidin (fibroblasts) or TUBB3 (SH-SY5Y cells and iPSC-neurons)-positive cell body. Sequential masks were generated for (1) the TGN by outlining the area covered by TGN marker TGN46 (TGN46-positive area, in fibroblasts and SH-SY5Y cells) or Golgi 97 (Golgi 97-positive area, in iPSC neurons) and (2) for the cell area outside the TGN (actin-positive area minus TGN46-positive area). ATG9A fluorescence intensity was measured in both compartments in each cell and the ATG9A ratio was calculated by dividing the ATG9A fluorescence intensity the TGN by the ATG9A fluorescence intensity in the remaining cell body (FIG. 1B). Z′-factor robust values and strictly standardized median difference (SSMD) were calculated for each plate and only plates that met the predefined quality metrics of a Z′-factor robust ≥0.3 and SSMD≥3 were included in subsequent analyses.

Western blotting. Western blotting was done. In brief, cells were lysed in RIPA Lysis Buffer (Thermo Fisher Scientific Cat #89900) supplemented with COMPLETE™ protease inhibitor (Cat #04693124001) and PHOSSTOP™ phosphatase inhibitor (Roche Cat #4906845001). Total protein concentration was determined using a Pierce BCA Protein Assay Kit (Thermo Fisher Scientific, #23225). Equal amounts of protein were solubilized in LDS sample buffer (Thermo Fisher Scientific, Cat #NP0008) under reducing conditions, separated by gel electrophoresis, using 4-12% (Thermo Fisher Scientific, Cat #NW04125BOX) or 12% Bis-Tris gels (Thermo Fisher Scientific, Cat #NP0343BOX) and MOPS or MES buffer (Thermo Fisher Scientific, #NP0001 and #NP0002) and transferred to a PVDF or nitrocellulose membranes (EMD Millipore, #SLHVR33RS). Following blocking with blocking buffer (LI-COR Biosciences, #927-70001), membranes were incubated overnight with the respective primary antibodies. Near-infrared fluorescent-labeled secondary antibodies (IR800CW, IR680LT; LI-COR Biosciences) were used and quantification was done using the Odyssey infrared imaging system and Image Studio Software (LI-COR Biosciences).

Sample preparation for RNA extraction. SH-SY5Y cells were differentiated with retinoic acid as described above and subsequently treated with compounds of interest for 72 hours, prior to lysis using the Quiagen RTL-Buffer supplemented with 1% β-mercaptoethanol. RNA extraction, library preparation and sequencing were conducted at Azenta Life Sciences (South Plainfield, NJ, USA). Total RNA was extracted from frozen cell pellet samples using Qiagen RNEASY™ mini kit following manufacturer's instructions (Qiagen, Hilden, Germany).

Library preparation with polyA selection and Illumina sequencing. RNA samples were quantified using Qubit 4 Fluorometer (Life Technologies, Carlsbad, CA, USA) and RNA integrity was checked using Agilent TapeStation 4200 (Agilent Technologies, Palo Alto, CA, USA). RNA sequencing libraries were prepared using the NEBNext Ultra II RNA Library Prep Kit for Illumina using manufacturer's instructions (NEB, Ipswich, MA, USA). Briefly, mRNAs were initially enriched with Oligod(T) beads. Enriched mRNAs were fragmented for 15 minutes at 94° C. First strand and second strand cDNA were subsequently synthesized. cDNA fragments were end repaired and adenylated at 3′ends, and universal adapters were ligated to cDNA fragments, followed by index addition and library enrichment by PCR with limited cycles. The sequencing library was validated on the Agilent TapeStation (Agilent Technologies, Palo Alto, CA, USA), and quantified by using Qubit 4 Fluorometer (Invitrogen, Carlsbad, CA) as well as by quantitative PCR (KAPA Biosystems, Wilmington, MA, USA). The sequencing libraries were clustered on 3 lanes of a flowcell. After clustering, the flowcell was loaded on the Illumina instrument (HiSeq 4000 or equivalent) according to manufacturer's instructions. The samples were sequenced using a 2×150 bp Paired End (PE) configuration. Image analysis and base calling were conducted by the Control software. Raw sequence data (.bcl files) generated the sequencer were converted into fastq files and de-multiplexed using Illumina's bcl2fastq 2.17 software. One mismatch was allowed for index sequence identification.

Downstream RNA sequencing analysis. Sequencing reads were mapped to the GRCh38 reference genome available on ENSEMBL using the STAR aligner v.2.7.9a. Differential expression analysis was done using the TREAT approach developed by McCarthy and Smyth (McCarthy D J, Smyth GK. Bioinformatics 25, 765-771 (2009)), implemented in the edgeR package in R. Raw counts were obtained using STAR and low expressed genes were excluded using the method described by Chen et al. (Chen Y, Lun A T, Smyth G K. F1000Res 5, 1438 (2016)). Expression data were normalized using the Trimmed Mean of M-values method implemented in the edgeR package. Genes were considered as differentially expressed according to default options with a false discovery rate (Benjamini-Hochberg procedure)<0.05 and a log 2 fold change of >0.3. Gene ontology (GO) enrichment analysis was done using clusterProfiler. Pathways were considered differentially expressed with an FDR<0.05.

Network connectivity analysis. To identify transcriptional changes in co-expressed groups of genes following compound treatment, a weighted gene co-expression network analysis (WGCNA) was performed. Raw counts were generated, and low expressed genes were removed as described above. Data were normalized using variance stabilizing transformation as described by Anders et al. (Anders S, Huber W. Genome Biol 11, R106 (2010)). Batch effects were removed using the limma package in R. Preprocessed data were then analyzed using the WGCNA package in R. In brief, pairwise Pearson correlations were calculated between all genes and genes with a positive correlation were selected to form a “directed” correlation matrix. Next, the correlations were raised to a power to approximate a scale free network. The adequate power was chosen based on soft thresholding aiming for a high scale independence above 0.8 by keeping a mean connectivity between 200 and 500. Genes were then grouped based on topological overlap and clusters were isolated using hierarchical clustering and adaptive branch pruning of the hierarchical cluster dendrogram, giving rise to groups of co-expressed genes, so called modules. Gene expression profiles within each module were summarized using the “module eigengene” (ME), defined as the first principal component of a module. Within each module, association of MEs with measured clinical traits was examined by correlation analysis. For these selected modules, module eigengene based connectivity was determined for every gene by calculating the absolute value of the Pearson correlation between the expression of the gene and the respective ME, producing a quantitative measure of module membership (MM). Similarly, the correlation of individual genes with the trait of interest was computed, defining gene significance (GS). Using the GS and MM, an intramodular analysis was performed, allowing identification of genes that have high significance with treatment as well as high connectivity to their modules. The biological information contained in modules of interest was summarized with gene ontology (GO) enrichment analysis using clusterProfiler. Pathways were considered differentially expressed with an FDR<0.05.

Sample preparation for mass spectrometry. Cells were lysed for whole proteome analysis in RIPA Lysis Buffer (Thermo Fisher Scientific, Cat #89900) supplemented with COMPLETE™ protease inhibitor (Cat #04693124001) and PHOSSTOP™ phosphatase inhibitor (Roche Cat #4906845001) and sonicated in a BIORUPTOR® Pico Sonication System (one single 30 seconds on/off cycle at 4° C.). Protein concentrations were determined using a Pierce BCA Protein Assay Kit (Thermo Fisher Scientific Cat #23225). Lysates were stored at −80° C. until further processing. To generate peptide samples for analysis by mass spectrometry, 30-50 μg protein were precipitated by overnight incubation in 5 volumes of ice-cold acetone at −20° C. and pelleted by centrifugation at 10,000×g for 5 minutes at 4° C. All subsequent steps were performed at room temperature. Precipitated protein pellets were air-dried, resuspended for denaturation and reduction in digestion buffer (50 mM Tris pH 8.3, 8M Urea, 1 mM dithiothreitol (DTT)) and incubated for 15 minutes. Proteins were alkylated by addition of 5 mM iodoacetamide for 20 minutes in the dark. Following reduction and alkylation, proteins were enzymatically digested by addition of LysC (1 pg per 50 μg of protein; Wako, Cat #129-02541) for an overnight incubation. Samples were then diluted four-fold with 50 mM Tris pH 8.3 before addition of Trypsin (1 μg per 50 μg of protein; Sigma-Aldrich, Cat #T6567) for 3 hours. The digestion reaction was stopped by addition of 1% (v/v) trifluoroacetic acid (TFA) and samples were incubated on ice for 5 minutes to precipitate contaminants, which were pelleted by centrifugation at 10,000×g for 5 minutes. Acidified peptides were transferred to new tubes, before purification by solid-phase extraction using poly(styrenedivinylbenzene) reverse-phase sulfonate (SDB-RPS; Sigma-Aldrich, Cat #66886-U) StageTips⁷⁶. StageTips with three SDB-RPS plugs were washed with 100% acetonitrile, equilibrated with StageTip equilibration buffer (30% [v/v] methanol, 1% [v/v] TFA), and washed with 0.2% (v/v) TFA. 20 μg of peptides in 1% TFA were then loaded onto the activated StageTips, washed with 100% isopropanol, and then 0.2% (v/v) TFA. Peptides were eluted in three consecutive fractions by applying a step gradient of increasing acetonitrile concentrations: 20 μL SDB-RPS-1 (100 mM ammonium formate, 40% [v/v] acetonitrile, 0.5% [v/v] formic acid), then 20 μL SDB-RPS-2 (150 mM ammonium formate, 60% [v/v] acetonitrile, 0.5% [v/v] formic acid), then 30 μL SDB-RPS-3 (5% [v/v] NH₄OH, 80% [v/v] acetonitrile). Eluted peptides were dried in a centrifugal vacuum concentrator, resuspended in Buffer A* (0.1% (v/v) TFA, 2% (v/v) acetonitrile), and stored at −20° C. until analysis by mass spectrometry.

Mass spectrometry. Mass spectrometry was performed on an Exploris 480 mass spectrometer coupled online to an EASY-nLC 1200, via a nano-electrospray ion source (all Thermo Fisher Scientific). Per sample, 250 ng of peptides were loaded on a 50 cm by 75 μm inner diameter column, packed in-house with ReproSil-Pur C18-AQ 1.9 μm silica beads (Dr Maisch GmbH). The column was operated at 50° C. using an in-house manufactured oven. Peptides were separated at a constant flow rate of 300 nL/minute using a linear 110 minute gradient employing a binary buffer system consisting of Buffer A (0.1% [v/v] formic acid) and Buffer B (80% acetonitrile, 0.1% [v/v] formic acid). The gradient ran from 5 to 30% B in 84 minutes, followed by an increase to 60% B in 8 minutes, a further increase to 95% B in 4 minutes, a constant phase at 95% B for 4 minutes, and then a washout decreasing to 5% B in 5 minutes, before re-equilibration at 5% B for 5 minutes. The Exploris 480 was controlled by Xcalibur software (v.4.4, Thermo Fisher Scientific) and data were acquired using a data-dependent top-15 method with a full scan range of 300-1650 Th. MS1 survey scans were acquired at 60,000 resolution with an automatic gain control (AGC) target of 3×10⁶ charges and a maximum ion injection time of 25 milliseconds. Selected precursor ions were isolated in a window of 1.4 Th and fragmented by higher-energy collisional dissociation (HCD) with normalized collision energies of 30. MS2 fragment scans were performed at 15,000 resolution, with an AGC target of 1×10⁵ charges, a maximum injection time of 28 milliseconds, and precursor dynamic exclusion for 30 seconds.

Raw mass spectrometry data analysis. Mass spectrometry raw files were processed in MaxQuant Version 2.1.4.0, using the human SwissProt canonical and isoform protein database, retrieved from UniProt (2022_09_26; uniprot.org). Label-free quantification was performed using the MaxLFQ algorithm. Matching between runs was enabled to match between equivalent and adjacent peptide fractions, within replicates. LFQ minimum ratio count was set to 1 and default parameters were used for all other settings. All downstream analyses were performed on the ‘protein groups’ file output from MaxQuant.

Proteomic downstream data analysis. Differential enrichment analysis of proteomics data was done using the DEP package in R. Preprocessing and quality filtering was performed separately for SH-SY5Y cells and iPSC-derived neurons. Proteins that were only identified by a modification site, or matched the reversed part of the decoy database, as well as commonly occurring contaminants were removed. Duplicate proteins were removed based on the corresponding gene names by keeping those with the highest total MS/MS count across all samples. All following steps were done separately for each cell type (SH-SY5Y cells (FIG. 7A and FIGS. 14A-14D) and iPSC-derived neurons (FIG. 7B and FIGS. 14E-14H) and for the pooled dataset (FIG. 7C and FIGS. 14I-14L). Low quality entries were removed by keeping only those proteins that had valid MS/MS counts in all replicate samples of at least one experimental condition. Finally, only those proteins were kept that had a maximum of one missing LFQ value in at least one experimental condition. Filtered data were normalized using variance stabilizing transformation and missing values were imputed using a manually defined left-shifted Gaussian distribution with a width of 0.3 and a left-shift of 2.2 SD. Batch effects were corrected using the method described by Johnson et al. (Johnson W E, Li C, Rabinovic A. Biostatistics 8, 118-127 (2007)). Statistical testing for differential protein enrichment was done using protein-wise linear models and empirical Bayes statistics implemented in the limma package in R. Proteins were considered as differentially enriched with a false discovery rate of <0.05 and a log 2 fold change >0.3. The biological information contained in differentially enriched proteins was summarized using Reactome pathway annotation in clusterProfiler. Pathways were considered differentially expressed with an FDR<0.05.

Electroporation. sgRNAs against NLRP5, RAB3C and RAB12 were purchased as multi-guide knockout kits from Synthego, diluted to the desired stock concentrations and kept at −80° C. Electroporation was performed under RNAse free conditions on a Lonza 4D-Nucleofector according to the manufacturer's protocol. Briefly, SH-SY5Y cells were harvested and resuspended in Nucleofector Solution at a concentration of 400×10\hat{ }3 cells/ml. sgRNAs were incubated with Cas9 protein to form ribonucleoprotein complexes (RNPs) according to the manufacturer's instructions. The cell solution was then incubated with an amount of the respective RNPs and transferred into a nucleofection cuvette. Cuvettes were placed in the 4D-Nucleofector System, and electroporation was done using the G-004 program. Following electroporation, pre-warmed medium was added, and cells were plated. Compound treatment was started 48 hours after electroporation. Knockout efficiency of sgRNAs was assessed using the Synthego ICE Analysis online tool. For this, genomic DNA was extracted from nucleofected cells using a **kit** according to manufacturer's instructions and sequenced using the following primers: NLRP5 forward:

NLRP5 forward:
CTTGAGAATTTGCTGCAAGATCCT,
NLRP5 reverse:
CGATTCTTCCCTGTTCCCATGAG,
RAB3C forward:
CCACTCGCCTCCTGAGTGTCTG,
RAB3C reverse:
GAACAAGGCAGAAAGTTTCTCCC,
RAB12 forward:
CGAGTAGGGAGGAGTGAAAAGG,
RAB12 reverse:
GGCACGAAAACCTCTGCCAGGC.

Statistical testing. Statistical analysis of continuous variables was performed with R version 4.2.1 (2022 Jun. 23) and RStudio (version 2022.07.1; RStudio, Inc.) using either mean and standard deviation (SD) or median and interquartile range (IQR), depending on the distribution of data tested by visualization with histograms, quantile-quantile plots and normality testing using the Shapiro-Wilk test. Sample sizes were indicated (n) for each analysis. The T-Test (for normally distributed variables) and the Mann-Whitney U test (for non-parametric distributions) was performed to test for statistical differences.

Assay performance metrics of the primary screen in AP-4-HSP patient-derived fibroblasts. Measurements of the ATG9A fluorescence (inside the TGN, outside the TGN, and the ATG9A ratio) on the level of individual cells (total of 219,448 cells) in fibroblasts from a patient with AP-4-HSP due to biallelic loss-of-function variants in AP4B1 (LoF/LoF) and their sex-matched parental control (WT/LoF) treated with vehicle (DMSO) served as negative (LoF/LoF) and positive controls (WT/LoF) on each of the 82 assay plates and allow for the calculation of standard metrics for assay performance. Robust separation of controls (FIGS. 1D-1K), and strong Z′ robust, SSMD, coefficient of variance support choice of the ATG9A ration as the primary readout for the primary screen.

Assay performance metrics of the primary screen in AP-4-HSP patient-derived fibroblasts. Assay performance was monitored using established criteria for cell-based assays with a Z′ robust ≥0.3, a strictly standardized median difference (SSMD)≥3 and an inter-assay coefficient of variation ≤10%. All assay metrics were calculated using the positive and negative controls of the same assay plate to avoid bias by inter-plate variability. Predefined thresholds were met by all assay plates.

Complete source data of the primary screen in AP-4-HSP patient-derived fibroblasts. For each experimental well (28,864 compounds and controls) in all 82 assay plates, the ATG9A fluorescence metrics (inside the TGN, outside the TGN, and the ATG9A ratio) and cell counts were automatically computed. Compounds were classified as ‘inactive’ (ATG9A ratio greater that −3SD), ‘active’ (ATG9A ratio equal or less than −3SD), ‘toxic’ (cell count equal or less than −2SD), ‘positive controls’ and ‘negative controls’.

Complete source data of the counter-screen in AP-4-HSP patient-derived fibroblasts. For each experimental well (503 compounds in an 11-point dose range and controls in biological duplicates) in 34 assay plates, the ATG9A fluorescence metrics (inside the TGN, outside the TGN, and the ATG9A ratio) and cell counts were automatically computed. Compounds were classified as ‘inactive’ (ATG9A ratio greater that −3SD), ‘active’ (reduced the ATG9A ratio by at least 3SD in both replicates and at least 2 different concentrations), ‘toxic’ (cell count equal or less than −2SD), ‘positive controls’ and ‘negative controls’.

Assay performance metrics of the counter-screen in AP-4-HSP patient-derived fibroblasts. Assay performance was monitored using established criteria for cell-based assays with a Z′ robust ≥0.3, a strictly standardized median difference (SSMD)≥3 and an inter-assay coefficient of variation ≤10%. All assay metrics were calculated using the positive and negative controls of the same assay plate to avoid bias by inter-plate variability. Predefined thresholds were met by all assay plates.

Complete source data of the orthogonal screen in AP4B1^{WT} and AP4B1^{KO} SH-SY5Y cells. To assess for dose-dependent effects in a neuronal model of AP-4-deficiency, the active compounds identified in the counter-screen were re-screened in differentiated AP4B1^{WT} and AP4B1^{KO} SH-SY5Y cells using an 8-point titration, ranging from 50 nM to 30 μM. Active compounds were a priori defined as those reducing the ATG9A ratio or DAGLB ratio by at least 3SD compared to negative controls, in more than one concentration. Toxicity was defined as a reduction of cell count of at least 2SD compared to the negative control.

### Compound Synthesis

Compounds of Formula (I) were prepared following the synthetic schemes and procedures described in detail below. The examples described in this application are offered to illustrate the compounds, pharmaceutical compositions, and methods provided herein and are not to be construed in any way as limiting their scope. Compounds of the disclosure that are not explicitly described in the following procedures may be prepared by analogous methods. Those having ordinary skill in the art would understand how to make such compounds from the disclosure provided herein and by means known in the art of organic synthesis. For example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof are representative and instructive. Methods for optimizing reaction conditions, if necessary minimizing competing by products, are known in the art. Embodiments of this disclosure include methods of synthesizing compounds delineated herein using any of the compounds, reactants, and/or processes delineated herein.

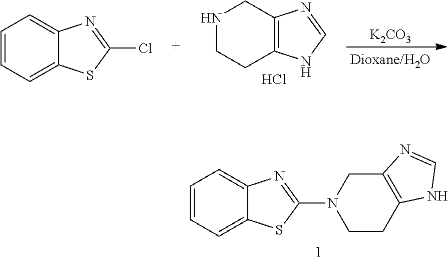

To a solution of 4,5,6,7-tetrahydro-1H-imidazo[4,5-c]pyridine (18.82 g, 117.90 mmol, 2 eq, HCl), K₂CO₃ (24.44 g, 176.85 mmol, 3 eq) in Dioxane (150 mL), H₂O (150 mL) was added 2-chloro-1,3-benzothiazole (10 g, 58.95 mmol, 7.67 mL, 1 eq) and stirred at 100° C. for 16 hr. LCMS (EW52308-8-P1A2) showed 4.7% of the reactant 1 was remained and 94% of desired mass was detected. The reaction mixture was concentrated under reduced pressure to remove Dioxane, extracted with EA (100 mL*3) and concentrated in vacuo to give a residue. The residue was purified by reversed-phase HPLC (water (FA)-ACN condition). Compound 1 (C-01) (11 g, 42.42 mmol, 71.96% yield, 98.856% purity) was obtained as a white solid. LCMS: MS (ESI) Retention time: 0.467 min, (M+1)+=257.1. ¹H NMR (400 MHz, DMSO-d₆) δ=2.77 (br t, J=5.64 Hz, 2H) 3.93 (t, J=5.76 Hz, 2H) 4.59 (s, 2H) 7.02-7.12 (m, 1H) 7.23-7.32 (m, 1H) 7.47 (d, J=7.84 Hz, 1H) 7.56 (s, 1H) 7.73-7.80 (m, 1H) 8.15 (s, 1H)

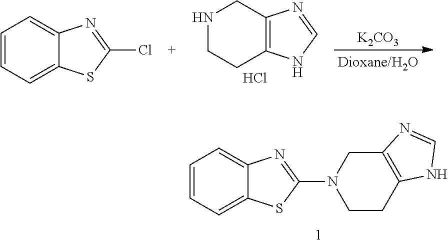

To a solution of Compound A (200 mg, 1.00 mmol, 1 eq) in DMF (1 mL) was added K₂CO₃ (415.33 mg, 3.01 mmol, 3 eq) and Compound B (148.04 mg, 1.20 mmol, 1.2 eq). The mixture was stirred at 40° C. for 12 hr. LC-MS showed desired mass was detected. The reaction mixture was filtered and concentrated under reduced pressure to give a solution. The solution was purified by reversed-phase HPLC (0.1% FA condition). 9 (100 mg, 349.22 mol, 34.86% yield, 93% purity) was obtained as a white solid. LCMS: MS (ESI) Retention time: 0.607 min (M+1)+=287.3. ¹H NMR (400 MHz, METHANOL-d₄) δ=8.26 (s, 1H), 7.81 (s, 1H), 7.26 (dd, J=0.8, 8.0 Hz, 1H), 7.07 (t, J=8.0 Hz, 1H), 6.90 (d, J=8.0 Hz, 1H), 4.69 (s, 2H), 4.00 (t, J=5.6 Hz, 2H), 3.96 (s, 3H), 2.87 (br t, J=5.6 Hz, 2H)

To a solution of Compound 9 (50 mg, 174.61 mol, 1 eq) in DCM (4 mL) was added BBr₃ (1 M, 349.22 μL, 2 eq) at −70° C. The mixture was stirred at 25° C. for 24 hr. LC-MS showed desired mass was detected. The reaction mixture was quenched by addition saturation NaHCO₃10 mL, then filtered and concentrated under reduced pressure to give a residue. The residue was purified by prep-HPLC (column: Phenomenex luna C18 150*25 mm*10 um; mobile phase: [water (FA)-ACN]; gradient: 8%-28% B over 9 min). Compound 10 (2.29 mg, 8.39 mol, 4.81% yield, 99% purity) was obtained as a white solid. LCMS: MS (ESI) Retention time: 0.472 min (M+1)+=273.1. ¹H NMR (400 MHz, METHANOL-d₄) δ=7.86 (br s, 1H), 7.17 (dd, J=1.0, 8.0 Hz, 1H), 6.96 (t, J=8.0 Hz, 1H), 6.77 (dd, J=0.8, 8.0 Hz, 1H), 4.72 (s, 2H), 4.01 (t, J=5.6 Hz, 2H), 2.89 (br t, J=5.6 Hz, 2H).

Compounds 11-14, 17-19, and 25-29 were prepared in an analogous manner.

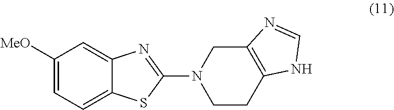 [SMILES: COc1ccc2sc(N3CCc4[nH]cnc4C3)nc2c1]

Compound 11: The residue was purified by prep-HPLC (0.1% FA condition) to afford desired compound (120 mg, 410.68 mol, 27.33% yield, 98% purity) as a white solid. LCMS: MS (ESI) Retention time: 0.398 min (M+1)+=287.1. ¹H NMR (400 MHz, CHLOROFORM-d) δ=7.63 (s, 1H), 7.47 (d, J=8.8 Hz, 1H), 7.16 (d, J=2.2 Hz, 1H), 6.91 (dd, J=2.4, 8.8 Hz, 1H), 4.67 (s, 2H), 4.00 (br t, J=5.6 Hz, 2H), 3.83 (s, 3H), 2.89 (br s, 2H).

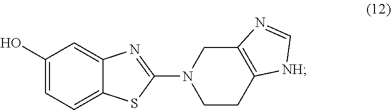 [SMILES: Oc1ccc2sc(N3CCc4[nH]cnc4C3)nc2c1]

Compound 12: The residue was purified by prep-HPLC (column: Phenomenex luna C18 150*25 mm*10 um; mobile phase: [water(FA)-ACN]; gradient: 1%-22% B over 10 min) to afford desired compound (19.44 mg, 71.39 mol, 40.88% yield, 100% purity) as a white solid. LCMS: MS (ESI) Retention time: 0.654 min (M+1)+=273.1. ¹H NMR (400 MHz, METHANOL-d₄) δ=8.22 (s, 1H), 7.85 (s, 1H), 7.33 (d, J=8.8 Hz, 1H), 7.09 (d, J=2.4 Hz, 1H), 6.79 (dd, J=2.4, 8.8 Hz, 1H), 4.65 (s, 2H), 3.94 (t, J=5.6 Hz, 2H), 2.87 (br t, J=5.6 Hz, 2H).

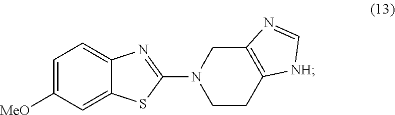 [SMILES: COc1ccc2nc(N3CCc4[nH]cnc4C3)sc2c1]

Compound 13: The residue was purified by prep-HPLC (0.1% FA condition) to afford desired compound (250 mg, 855.59 mol, 34.16% yield, 99% purity) as a white solid. LCMS: MS (ESI) Retention time: 0.559 min (M+1)+=287.3. ¹H NMR (400 MHz, CHLOROFORM-d) δ=7.59 (s, 1H), 7.47 (d, J=8.8 Hz, 1H), 7.17 (d, J=2.4 Hz, 1H), 6.91 (dd, J=2.4, 8.8 Hz, 1H), 4.67 (s, 2H), 4.01 (t, J=5.6 Hz, 2H), 3.83 (s, 3H), 2.89 (br t, J=5.6 Hz, 2H)

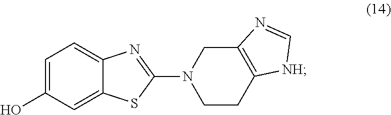 [SMILES: Oc1ccc2nc(N3CCc4[nH]cnc4C3)sc2c1]

Compound 14: The residue was purified by prep-HPLC (column: Phenomenex luna C18 150*25 mm*10 um; mobile phase:[water(FA)-ACN]; gradient: 1%-22% B over 10 min) to afford desired compound (2.22 mg, 8.07 mol, 2.31% yield, 99% purity) as a white solid. LCMS: MS (ESI) Retention time: 0.688 min (M+1)+=273.1. ¹H NMR (400 MHz, METHANOL-d₄) δ=8.26 (s, 1H), 7.79 (s, 1H), 7.33 (d, J=8.8 Hz, 1H), 7.08 (d, J=2.4 Hz, 1H), 6.78 (dd, J=2.4, 8.8 Hz, 1H), 4.63 (s, 2H), 3.94 (t, J=5.6 Hz, 2H), 2.86 (br t, J=5.6 Hz, 2H).

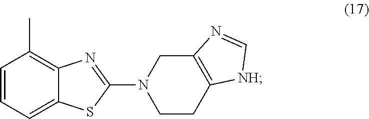 [SMILES: Cc1cccc2sc(N3CCc4[nH]cnc4C3)nc12]

Compound 17: The residue was purified by prep-HPLC (column: Phenomenex luna C18 150*25 mm*10 um; mobile phase: [water(FA)-ACN]; gradient: 12%-36% B over 8 min) to afford desired compound (68.55 mg, 253.56 mol, 46.57% yield, 100% purity) as a white solid. LCMS: MS (ESI) Retention time: 0.632 min (M+1)+=271.4. ¹H NMR (400 MHz, CHLOROFORM-d) δ=7.60 (s, 1H), 7.46 (d, J=7.2 Hz, 1H), 7.12 (br d, J=7.2 Hz, 1H), 7.04-6.95 (m, 1H), 4.73 (s, 2H), 4.06 (br t, J=5.6 Hz, 2H), 2.90 (br t, J=5.2 Hz, 2H), 2.58 (s, 3H).

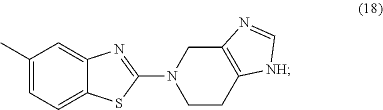 [SMILES: Cc1ccc2sc(N3CCc4[nH]cnc4C3)nc2c1]

Compound 18: The residue was purified by prep-HPLC (column: Phenomenex luna C18 150*25 mm*10 um; mobile phase: [water(FA)-ACN]; gradient: 9%-39% B over 10 min) to afford desired compound (56.41 mg, 208.65 mol, 38.32% yield, 100% purity) as a white solid. LCMS: MS (ESI) Retention time: 0.637 min (M+1)+=271.1. ¹H NMR (400 MHz, CHLOROFORM-d) δ=7.63 (br d, J=1.6 Hz, 1H), 7.56-7.46 (m, 1H), 7.44-7.35 (m, 1H), 7.04-6.85 (m, 1H), 4.70 (br d, J=7.6 Hz, 2H), 4.06 (br d, J=5.2 Hz, 2H), 2.98-2.84 (m, 2H), 2.51-2.38 (m, 3H).

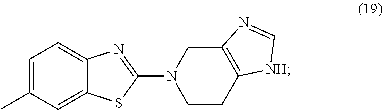 [SMILES: Cc1ccc2nc(N3CCc4[nH]cnc4C3)sc2c1]

Compound 19: The residue was purified by prep-HPLC (0.1% FA condition) to afford desired compound (31.45 mg, 110.51 mol, 10.15% yield, 94% purity) as a yellow solid. LCMS: MS (ESI) Retention time: 0.628 min (M+1)+=271.4. ¹H NMR (400 MHz, METHANOL-d₄) δ=7.72 (s, 1H), 7.47 (s, 1H), 7.38 (d, J=8.4 Hz, 1H), 7.12 (dd, J=1.2, 8.4 Hz, 1H), 4.66 (s, 2H), 3.97 (t, J=5.6 Hz, 2H), 2.86 (t, J=5.6 Hz, 2H), 2.38 (s, 3H)

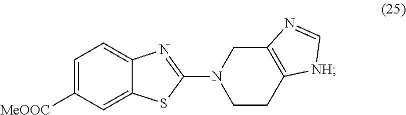 [SMILES: COC(=O)c1ccc2nc(N3CCc4[nH]cnc4C3)sc2c1]

Compound 25: The residue was purified by prep-HPLC (0.1% FA condition) to afford desired compound (600 mg, 1.91 mmol, 46.15% yield, 100% purity) as a white solid. LCMS: MS (ESI) Retention time: 0.612 min (M+1)+=315.3. ¹H NMR (400 MHz, CHLOROFORM-d) δ=8.33 (br s, 1H), 8.09-7.97 (m, 1H), 7.69-7.44 (m, 2H), 4.74 (br s, 2H), 4.21-4.04 (m, 2H), 3.92 (br s, 3H), 3.00-2.79 (m, 2H).

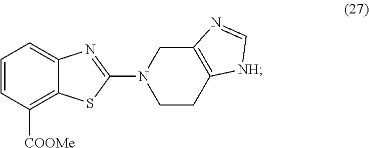 [SMILES: COC(=O)c1cccc2nc(N3CCc4[nH]cnc4C3)sc12]

Compound 27: The residue was purified by prep-HPLC (0.1% FA condition) to afford desired compound (39.20 mg, 120.96 mol, 55.08% yield, 96% purity) as an off-white solid. LCMS: MS (ESI) Retention time: 0.641 min (M+1)+=315.3. ¹H NMR (400 MHz, CHLOROFORM-d) δ=7.79 (d, J=7.6 Hz, 1H), 7.73 (d, J=8.0 Hz, 1H), 7.64 (s, 1H), 7.37 (t, J=8.0 Hz, 1H), 4.79 (s, 2H), 4.07 (br t, J=5.6 Hz, 2H), 3.99 (s, 3H), 2.92 (br s, 2H)

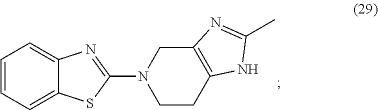 [SMILES: Cc1nc2c([nH]1)CCN(c1nc3ccccc3s1)C2]

Compound 29: The residue was purified by prep-HPLC (column: Phenomenex luna C18 150*25 mm*10 um; mobile phase: [water (FA)-ACN]; gradient: 12%-36% B over 8 min) to afford desired compound (16.47 mg, 59.09 mol, 20.05% yield, 97% purity) as a white solid. LCMS: MS (ESI) Retention time: 0.454 min (M+1)+=271.1. ¹H NMR (400 MHz, CHLOROFORM-d) δ=8.63 (s, 1H), 7.67-7.48 (m, 2H), 7.33-7.28 (m, 1H), 7.09 (t, J=7.6 Hz, 1H), 4.70 (s, 2H), 3.96 (t, J=5.6 Hz, 2H), 2.87 (br t, J=5.0 Hz, 2H), 2.51 (s, 3H).

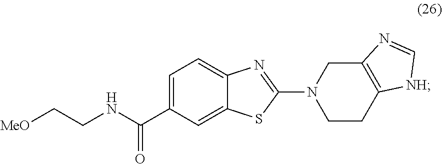 [SMILES: COCCNC(=O)c1ccc2nc(N3CCc4[nH]cnc4C3)sc2c1]

To a solution of Compound 25 (100 mg, 318.10 mol, 1 eq) in THF (2 mL) was added 3, 4, 6, 7, 8, 9-hexahydro-2H-pyrimido[1, 2-a]pyrimidine (221.40 mg, 1.59 mmol, 5 eq) and 2-methoxyethanamine (28.67 mg, 381.73 mol, 33.18 μL, 1.2 eq). The mixture was stirred at 25° C. for 12 hrs. The residue was purified by prep-HPLC (column: Welch Ultimate XB-SiOH 250*50*10 um; mobile phase: [Hexane-EtOH]; gradient: 15%-55% B over 15 min) to afford desired compound (24.09 mg, 64.03 mol, 20.13% yield, 95% purity) as a yellow solid. LCMS: MS (ESI) Retention time: 0.547 min (M+1)+=358.3. ¹H NMR (400 MHz, METHANOL-d₄) δ=8.10 (d, J=2.0 Hz, 1H), 7.93 (s, 1H), 7.71 (dd, J=2.0, 8.4 Hz, 1H), 7.43 (d, J=8.4 Hz, 1H), 4.68 (s, 2H), 3.96 (t, J=5.6 Hz, 2H), 3.50 (s, 3H), 3.24 (td, J=1.6, 3.2 Hz, 4H), 2.84 (br t, J=5.6 Hz, 2H).

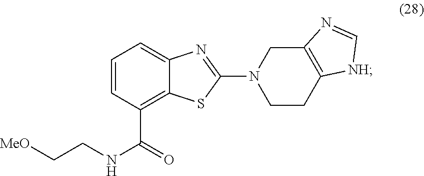 [SMILES: COCCNC(=O)c1cccc2nc(N3CCc4[nH]cnc4C3)sc12]

To a solution of Compound 27 (100 mg, 318.10 mol, 1 eq) in THF (2 mL) was added 3, 4, 6, 7, 8, 9-hexahydro-2H-pyrimido[1, 2-a]pyrimidine (221.40 mg, 1.59 mmol, 5 eq) and 2-methoxyethanamine (28.67 mg, 381.73 mol, 33.18 μL, 1.2 eq). The mixture was stirred at 25° C. for 12 hrs. The residue was purified by prep-HPLC (column: Welch Ultimate XB-SiOH 250*50*10 um; mobile phase: [Hexane-EtOH]; gradient: 15%-55% B over 15 min) to afford desired compound (30 mg, 75.54 mol, 47.49% yield, 96% purity) as a white solid. LCMS: MS (ESI) Retention time: 0.441 min (M+1)+=358.1. ¹H NMR (400 MHz, METHANOL-d₄) δ=8.25 (s, 1H), 7.91 (s, 1H), 7.61 (t, J=7.2 Hz, 2H), 7.37 (t, J=8.0 Hz, 1H), 4.72 (s, 2H), 4.00 (t, J=5.6 Hz, 2H), 3.59 (s, 4H), 3.38 (s, 3H), 2.88 (br t, J=5.6 Hz, 2H).

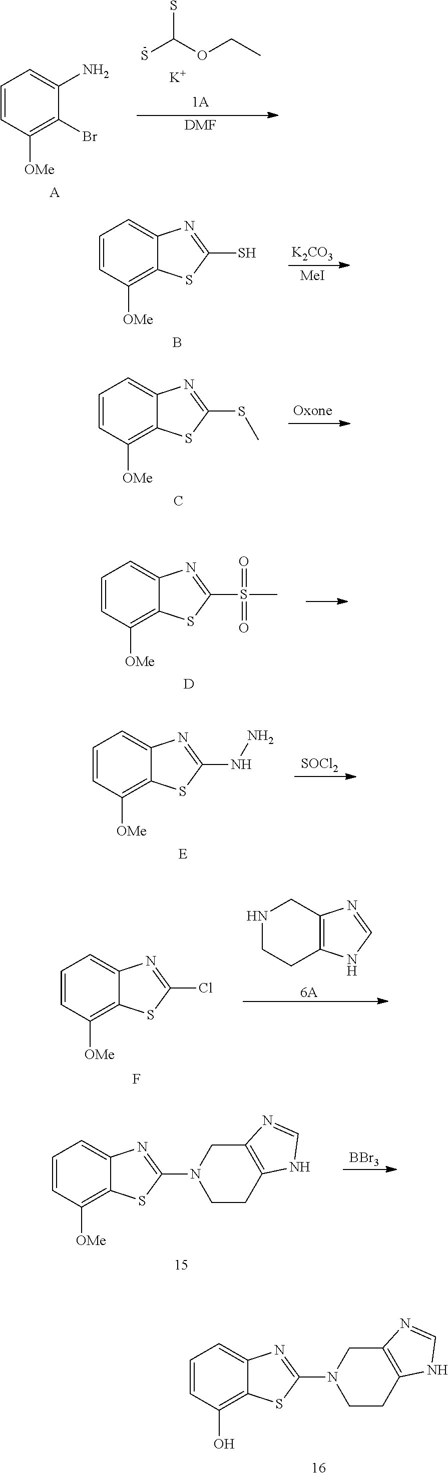

To a solution of Compound A (3 g, 14.85 mmol, 1 eq) in DMF (20 mL) was added Compound 1A (7.14 g, 44.54 mmol, 3 eq). The mixture was stirred at 160° C. for 4 hrs. LC-MS showed desired mass was detected. The reaction mixture was adjusted pH to 5, and then diluted with H₂O 50 mL and extracted with EA 300 mL (100 mL*3). The combined organic Layers were washed with brine 200 mL (100 mL*2), dried over Na₂SO₄, filtered and concentrated under reduced pressure to give a residue. The residue was purified by column chromatography (SiO₂, Petroleum ether/Ethyl acetate=10/1 to 0/1). Compound B (1.5 g, 6.84 mmol, 46.09% yield) was obtained as a white solid. LCMS: MS (ESI) Retention time: 0.695 min (M+1)+=198.4.

To a solution of Compound B (1.5 g, 7.60 mmol, 1 eq) in DMF (15 mL) was added K₂CO₃ (3.15 g, 22.81 mmol, 3 eq) and Mel (1.40 g, 9.88 mmol, 615.35 μL, 1.3 eq). The mixture was stirred at 30° C. for 12 hrs, LC-MS showed desired mass was detected. The reaction mixture was diluted with H₂O 200 mL and extracted with EA 150 mL (50 mL*3). The combined organic Layers were washed with brine 300 mL (150 mL*2), dried over Na₂SO₄, filtered and concentrated under reduced pressure to give a residue. The residue was purified by column chromatography (SiO₂, Petroleum ether/Ethyl acetate=20/1 to 2/1). Compound C (1.3 g, 6.15 mmol, 80.91% yield) was obtained as a white solid. LCMS: MS (ESI) Retention time: 0.595 min (M+1)+=212.2. ¹H NMR (400 MHz, METHANOL-d4) δ=7.44-7.36 (m, 2H), 6.88 (dd, J=2.0, 7.2 Hz, 1H), 3.96 (s, 3H), 2.78 (s, 3H).

To a solution of Compound C (1.3 g, 6.15 mmol, 1 eq) in THF (10 mL) and H₂O (5 mL) was added Oxone (11.35 g, 18.46 mmol, 3 eq). The mixture was stirred at 30° C. for 12 hrs. LC-MS showed desired mass was detected. the reaction mixture was diluted with H₂O 50 mL and extracted with EA 300 mL (100 mL*3). The combined organic Layers were washed with brine 200 mL (100 mL*2), dried over Na₂SO₄, filtered and concentrated under reduced pressure to give a residue, The residue was used to the next step without purification. Compound D (1.5 g, crude) was obtained as a yellow solid. LCMS: MS (ESI) Retention time: 0.723 min (M+1)+=243.9. ¹H NMR (400 MHz, CHLOROFORM-d) δ=7.81 (d, J=8.4 Hz, 1H), 7.57 (t, J=8.0 Hz, 1H), 6.99 (d, J=8.0 Hz, 1H), 4.03 (s, 3H), 3.40 (s, 3H).

To a solution of Compound D (1.5 g, 6.17 mmol, 1 eq) in EtOH (10 mL) was added hydrazine hydrate (9.26 g, 184.96 mmol, 8.97 mL, 100% purity, 30 eq) under N₂. The mixture was stirred at 100° C. for 1 h, LC-MS showed desired mass was detected. The reaction mixture was, filtered and concentrated under reduced pressure to give a residue. The residue was used to the next step without purification. Compound E (880 mg, 4.51 mmol, 73.11% yield) was obtained as a yellow solid. LCMS: MS (ESI) Retention time: 0.289 min (M+1)+=296.1. ¹H NMR (400 MHz, DMSO-d6) δ=7.20-7.11 (m, 1H), 6.96 (dd, J=0.8, 8.0 Hz, 1H), 6.63 (d, J=7.6 Hz, 1H), 5.02 (br s, 2H), 3.86 (s, 3H).

To a solution of Compound E (780 mg, 4.00 mmol, 1 eq) in DCM (5 mL) was added SOCl₂ (7.13 g, 59.93 mmol, 4.35 mL, 15 eq). The mixture was stirred at 55° C. for 0.5 hr. LC-MS showed desired mass was detected. The reaction mixture was adjusted pH to 8 with saturation NaHCO₃, and then diluted with H₂O 20 mL and extracted with EA 300 mL (100 mL*2). The combined organic layers were washed with brine 100 mL (50 mL*2), dried over Na₂SO₄, filtered and concentrated under reduced pressure to give a residue. Then residue was used to the next step without purification. Compound F (600 mg, 3.01 mmol, 75.22% yield) was obtained as a yellow solid. LCMS: MS (ESI) Retention time: 0.633 min (M+1)+=200.1. ¹H NMR (400 MHz, CHLOROFORM-d) δ=7.58 (d, J=8.0 Hz, 1H), 7.44 (t, J=8.0 Hz, 1H), 6.87 (d, J=8.0 Hz, 1H), 3.99 (s, 3H)

To a solution of Compound F (300 mg, 1.50 mmol, 1 eq) in DMF (2 mL) was added K₂CO₃ (622.99 mg, 4.51 mmol, 3 eq) and Compound 6 A (222.06 mg, 1.80 mmol, 1.2 eq). The mixture was stirred at 40° C. for 12 hrs. LC-MS showed desired mass was detected. The reaction mixture was filtered and concentrated under reduced pressure to give a solution, the crude product was purified by reversed-phase HPLC (0.1% FA condition). Compound 15 (120 mg, 419.07 mol, 27.89% yield, 99% purity) was obtained as a white solid. LCMS: MS (ESI) Retention time: 0.516 min (M+1)+=287.1. ¹H NMR (400 MHz, METHANOL-d₄) δ=7.73 (s, 1H), 7.32-7.20 (m, 1H), 7.14 (dd, J=0.8, 8.0 Hz, 1H), 6.71 (d, J=7.6 Hz, 1H), 4.68 (s, 2H), 3.99 (t, J=5.6 Hz, 2H), 3.93 (s, 3H), 2.87 (t, J=6.0 Hz, 2H).

To a solution of Compound 15 (100 mg, 349.22 mol, 1 eq) in DCM (7 mL) was added BBr₃ (1 M, 698.44 μL, 2 eq) at −70° C. under N₂. The mixture was stirred at 0° C. for 2 hrs. LC-MS showed desired mass was detected. The reaction mixture was adjusted to pH 8 with saturated NaHCO₃, then filtered and concentrated under reduced pressure to give a residue. The residue was purified by prep-HPLC (column: Phenomenex Luna C18 150*25 mm*10 um; mobile phase: [water (FA)-ACN]; gradient: 11%-30% B over 10 min) to provide Compound 16 (22.26 mg, 81.74 mol, 23.41% yield, 99% purity) as a white solid. LCMS: MS (ESI) Retention time: 0.639 min (M+1)+=273.0. ¹H NMR (400 MHz, METHANOL-d4) δ=7.83 (s, 1H), 7.15-7.10 (m, 1H), 7.06-7.02 (m, 1H), 6.54 (dd, J=0.8, 8.0 Hz, 1H), 4.69 (s, 2H), 3.99 (t, J=5.6 Hz, 2H), 2.89 (t, J=5.6 Hz, 2H).

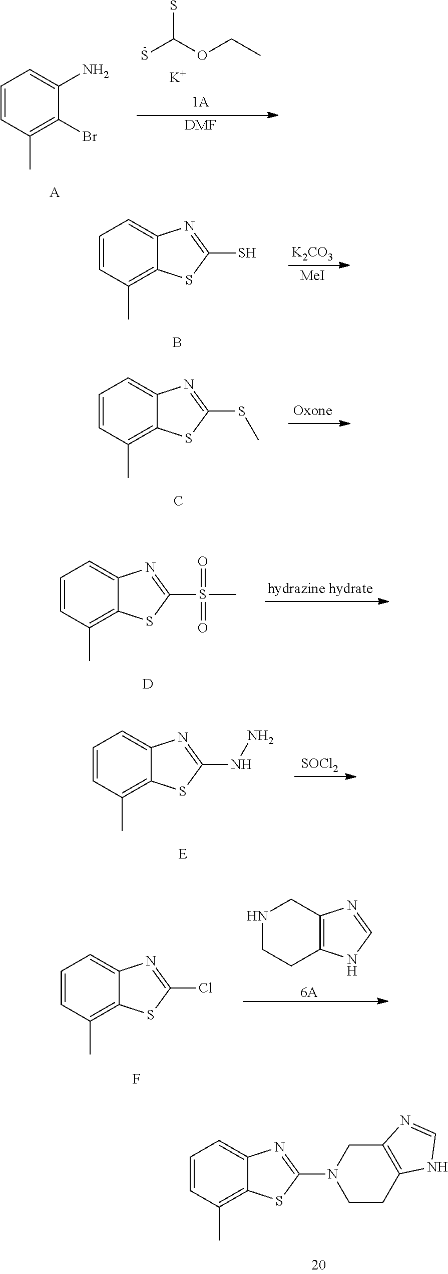

To a solution of Compound A (5 g, 26.87 mmol, 1 eq) in DMF (30 mL) was added Compound 1A (12.92 g, 80.62 mmol, 3 eq). The mixture was stirred at 160° C. for 4 hrs. LC-MS showed desired mass was detected. The reaction mixture was quenched by addition HCl (1M) 10 mL, and then diluted with H₂O 50 mL and extracted with EA 500 mL (250 mL*2). The combined organic Layers were washed with brine 100 mL (50 mL*2), dried over Na₂SO₄, filtered and concentrated under reduced pressure to give a residue. The residue was purified by column chromatography (SiO₂, Petroleum ether/Ethyl acetate=20/1 to 3/1). Compound B (4.8 g, crude) was obtained as yellow oil. LCMS: MS (ESI) Retention time: 0.555 min (M+1)+=182.1. ¹H NMR (400 MHz, DMSO-d₆) δ=13.76 (br s, 1H), 7.35-7.28 (m, 1H), 7.17-7.10 (m, 2H), 2.33 (s, 3H).

To a solution of Compound B (4.8 g, 26.48 mmol, 1 eq) in DMF (25 mL) was added Mel (7.52 g, 52.96 mmol, 3.30 mL, 2 eq). The mixture was stirred at 25° C. for 1 hr. Then K₂CO₃ (10.98 g, 79.44 mmol, 3 eq) was added. The mixture was stirred at 25° C. for 11 hrs. LC-MS showed desired mass was detected, the reaction mixture was diluted with H₂O 50 mL and extracted with EA 300 mL (150 mL*2). The combined organic layers were washed with brine 300 mL (100 mL*3), dried over Na₂SO₄, filtered and concentrated under reduced pressure to give a residue. The residue was purified by column chromatography (SiO₂, Petroleum ether/Ethyl acetate=20/1 to 3/1). Compound C (5.6 g, crude) was obtained as yellow oil. LCMS: MS (ESI) Retention time: 0.916 min (M+1)+=196.1.

To a solution of Compound C (5 g, 25.60 mmol, 1 eq) in THF (50 mL) and H₂O (15 mL) was added Oxone (31.48 g, 51.20 mmol, 2 eq). The mixture was stirred at 25° C. for 12 hrs. LC-MS showed desired mass was detected. The reaction mixture was concentrated under reduced pressure to remove THF, and then diluted with H₂O 50 mL and extracted with EA 300 mL (150 mL*2). The combined organic Layers were washed with brine 200 mL (100 mL*2), dried over Na₂SO₄, filtered and concentrated under reduced pressure to give a residue. The residue was used to the next step without purification, Compound D (4.6 g, 20.24 mmol, 79.05% yield) was obtained as a white solid. Confirmed by HNMR (EW43310-71-P1A). LCMS: MS (ESI) Retention time: 0.723 min (M+1)+=228.0. ¹H NMR (400 MHz, CHLOROFORM-d) δ=8.05 (d, J=8.4 Hz, 1H), 7.57 (t, J=8.0 Hz, 1H), 7.39 (d, J=7.6 Hz, 1H), 3.42 (s, 3H), 2.64 (s, 3H).

To a solution of Compound D (2.3 g, 10.12 mmol, 1 eq) in EtOH (15 mL) was added hydrazine hydrate (15.20 g, 303.56 mmol, 14.73 mL, 100% purity, 30 eq) under N₂. The mixture was stirred at 100° C. for 1 hr. LC-MS showed desired mass was detected. The reaction mixture was filtered and concentrated under reduced pressure to give a residue. Compound E (1.6 g, crude) was obtained as a white solid. LCMS: MS (ESI) Retention time: 0.298 min (M+1)+=180.1. ¹H NMR (400 MHz, METHANOL-d4) δ=7.24-7.20 (m, 1H), 7.19-7.13 (m, 1H), 6.87 (d, J=7.2 Hz, 1H), 2.42 (s, 3H).

To a solution of Compound E (1 g, 5.58 mmol, 1 eq) in DCM (6 mL) was added SOCl₂ (9.96 g, 83.69 mmol, 6.08 mL, 15 eq). The mixture was stirred at 55° C. for 0.5 hr.

LC-MS showed desired mass was detected. The reaction mixture was adjusted pH to 8 with saturation NaHCO₃, and then diluted with H₂O 20 mL and extracted with EA 300 mL (100 mL*2). The combined organic Layers were washed with brine 100 mL (50 mL*2), dried over Na₂SO₄, filtered and concentrated under reduced pressure to give a residue, The residue was used to the next step without purification, Compound F (800 mg, 4.36 mmol, 78.08% yield) was obtained as a yellow solid. LCMS: MS (ESI) Retention time: 0.612 min (M+1)+=184.0. ¹H NMR (400 MHz, METHANOL-d4) δ=7.72 (d, J=8.4 Hz, 1H), 7.43 (t, J=8.0 Hz, 1H), 7.26 (d, J=7.6 Hz, 1H), 2.50 (s, 3H).

To a solution of Compound F (200 mg, 1.09 mmol, 1 eq) in DMF (4 mL) was added K₂CO₃ (451.51 mg, 3.27 mmol, 3 eq) and Compound 6A (160.94 mg, 1.31 mmol, 1.2 eq). The mixture was stirred at 60° C. for 12 hrs. LC-MS showed desired mass was detected. The reaction mixture was filtered and concentrated under reduced pressure to give a solution. The residue was purified by prep-HPLC (column: Phenomenex Luna C18 150*25 mm*10 um; mobile phase: [water (FA)-ACN]; gradient: 12%-32% B over 9 min). Compound 20 (87.12 mg, 315.80 mol, 29.00% yield, 98% purity) was obtained as a white solid. LCMS: MS (ESI) Retention time: 0.751 min (M+1)+=271.4. ¹H NMR (400 MHz, METHANOL-d4) δ=7.79 (s, 1H), 7.35 (d, J=8.0 Hz, 1H), 7.22 (t, J=8.0 Hz, 1H), 6.93 (d, J=7.6 Hz, 1H), 4.71 (s, 2H), 4.01 (t, J=5.6 Hz, 2H), 2.89 (t, J=5.6 Hz, 2H), 2.45 (s, 3H).

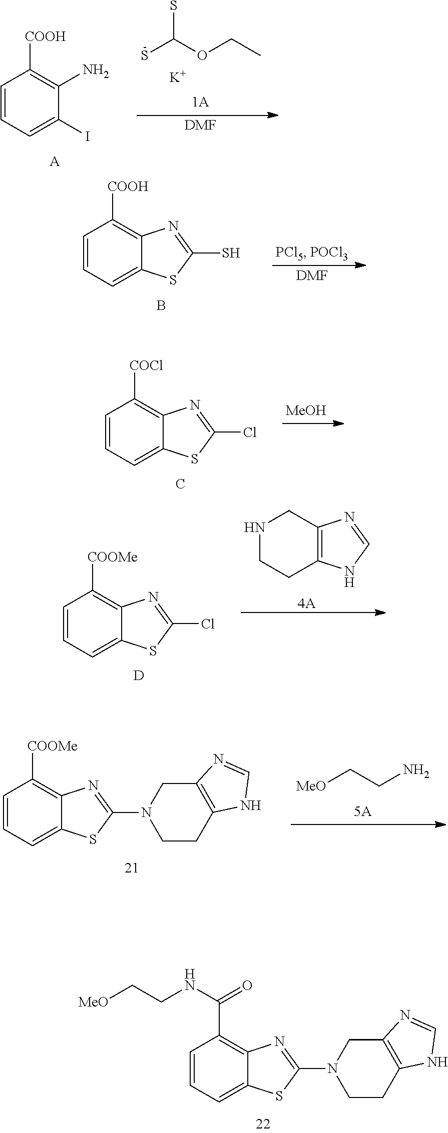

To a solution of Compound A (2 g, 7.60 mmol, 1 eq) in DMF (15 mL) was added Compound 1A (3.66 g, 22.81 mmol, 3 eq). The mixture was stirred at 160° C. for 4 hrs, LC-MS showed desired mass was detected, the reaction mixture was adjusted pH to 5 with HCl (1 M), and then diluted with H₂O 50 mL and extracted with EA 300 mL (100 mL*3). The combined organic Layers were washed with brine 200 mL (100 mL*2), dried over Na₂SO₄, filtered and concentrated under reduced pressure to give a residue. The residue was purified by column chromatography (SiO₂, Petroleum ether/Ethyl acetate=10/1 to 3/1). Compound B (600 mg, 2.84 mmol, 37.35% yield) was obtained as a yellow solid. LCMS: MS (ESI) Retention time: 0.652 min, (M+1)+=212.3. ¹H NMR (400 MHz, DMSO-d₆) δ=12.73-11.94 (m, 1H), 7.97-7.88 (m, 2H), 7.40 (t, J=8.0 Hz, 1H).

To a solution of Compound B (300 mg, 1.42 mmol, 1 eq) in DMF (0.5 mL) and POCl₃ (8.23 g, 53.64 mmol, 5 mL, 37.77 eq) was added PCl₅ (887.13 mg, 4.26 mmol, 3 eq). The mixture was stirred at 100° C. for 3 hrs. The reaction mixture was filtered and concentrated under reduced pressure to give a solution. The solution was used to the next step without purification, Compound 3 (300 mg, 1.29 mmol, 91.03% yield) was obtained as black oil.

A solution of Compound C (200 mg, 861.75 mol, 1 eq) in MeOH (20 mL) was stirred at 25° C. for 0.5 hr. LC-MS showed desired mass was detected. The reaction mixture was filtered and concentrated under reduced pressure to give a residue. The residue was purified by prep-HPLC (column: Phenomenex Luna C18 150*25 mm*10 um; mobile phase: [water (FA)-ACN]; gradient: 36%-66% B over 10 min). Compound D (60 mg, 255.64 mol, 29.66% yield, 97% purity) was obtained as a white solid. LCMS: MS (ESI) Retention time: 0.521 min (M+1)+=228.3.

To a solution of Compound D (60 mg, 263.54 mol, 1 eq) in DMF (1 mL) was added K₂CO₃ (109.27 mg, 790.63 mol, 3 eq) and Compound 4A (48.69 mg, 395.31 mol, 1.5 eq). The mixture was stirred at 40° C. for 12 hrs. LC-MS showed desired mass was detected. The reaction mixture was filtered and concentrated under reduced pressure to give a solution. The solution was purified by prep-HPLC (column: Phenomenex Luna C18 150*25 mm*10 um; mobile phase: [water (FA)-ACN]; gradient: 7%-37% B over 10 min). Compound 21 (50 mg, 158.89 mol, 60.29% yield, 99% purity) was obtained as an off-white solid. LCMS: MS (ESI) Retention time: 0.604 min (M+1)+=315.3. ¹H NMR (400 MHz, METHANOL-d₄) δ=7.92-7.80 (m, 3H), 7.13 (t, J=8.0 Hz, 1H), 4.77 (s, 2H), 4.07 (t, J=6.0 Hz, 2H), 3.94 (s, 3H), 2.89 (t, J=5.6 Hz, 2H).

To a solution of Compound 21 (40 mg, 127.24 mol, 1 eq) in THF (1 mL) was added 3, 4, 6, 7, 8, 9-hexahydro-2H-pyrimido[1, 2-a]pyrimidine (88.56 mg, 636.21 mol, 5 eq) and Compound 5A (14.34 mg, 190.86 mol, 16.59 μL, 1.5 eq). The mixture was stirred at 30° C. for 12 hrs. LC-MS showed desired mass was detected. The reaction mixture was filtered and concentrated under reduced pressure to give a residue. The residue was purified by prep-HPLC (column: Phenomenex Luna C18 150*25 mm*10 um; mobile phase: [water (FA)-ACN]; gradient: 12%-32% B over 10 min). Compound 22 (4.57 mg, 12.66 mol, 9.95% yield, 99% purity) was obtained as a white solid. LCMS: MS (ESI) Retention time: 0.459 min (M+1)+=358.2. ¹H NMR (400 MHz, METHANOL-d₄) δ=10.70-10.54 (m, 1H), 8.16-8.03 (m, 1H), 7.85 (dd, J=1.2, 7.2 Hz, 1H), 7.73-7.58 (m, 1H), 7.25-7.15 (m, 1H), 4.74 (br s, 2H), 4.19-4.06 (m, 2H), 3.69 (dt, J=4.8, 9.2 Hz, 4H), 3.51-3.44 (m, 3H), 2.97-2.89 (m, 2H).

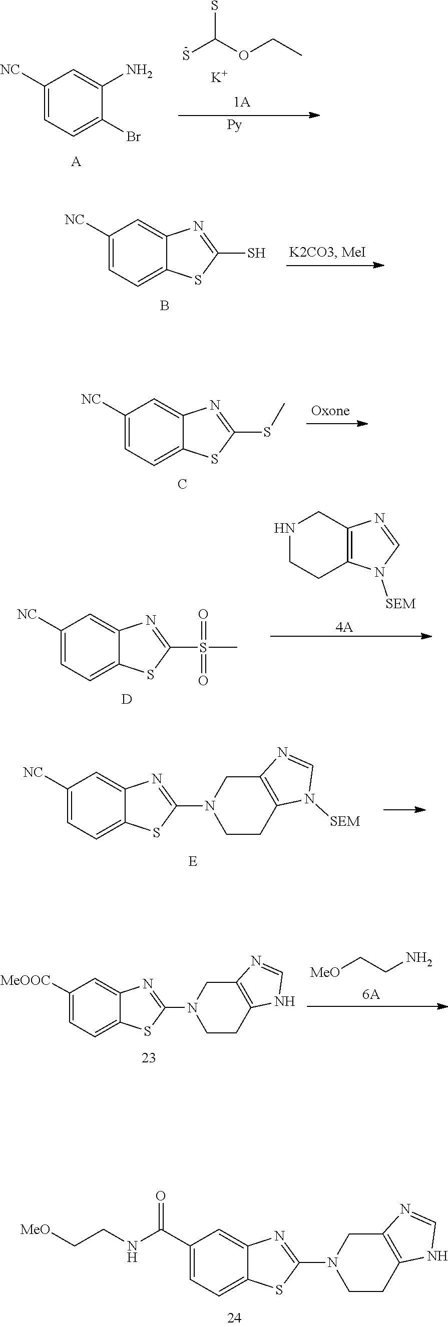

To a solution of Compound A (3 g, 15.23 mmol, 1 eq) in DMF (20 mL) was added Compound 1A (7.32 g, 45.68 mmol, 3 eq). The mixture was stirred at 160° C. for 4 hrs. LC-MS showed desired mass was detected, the reaction mixture was adjust pH to 5 with HCl (1 M), and then diluted with H₂O 50 mL and extracted with EA 300 mL (100 mL*3). The combined organic layers were washed with brine 200 mL (100 mL*2), dried over Na₂SO₄, filtered and concentrated under reduced pressure to give a residue. The residue was purified by column chromatography (SiO₂, Petroleum ether/Ethyl acetate=20/1 to 2/1). Compound B (3 g, crude) was obtained as a yellow solid. LCMS: MS (ESI) Retention time: 0.526 min (M+1)+=193.1. ¹H NMR (400 MHz, CHLOROFORM-d) δ=8.10 (s, 1H), 7.59-7.48 (m, 2H).

To a solution of Compound B (3 g, 15.60 mmol, 1 eq) in DMF (20 mL) was added Mel (4.43 g, 31.21 mmol, 1.94 mL, 2 eq) and K₂CO₃ (6.47 g, 46.81 mmol, 3 eq). The mixture was stirred at 25° C. for 12 hrs. LC-MS showed desired mass was detected. The reaction mixture was quenched by addition NH₄Cl 50 mL, and then diluted with 100 mL H₂O and extracted with EA 600 mL (200 mL*3). The combined organic Layers were washed with brine 200 mL (100 mL*2), dried, over Na₂SO₄, filtered and concentrated under reduced pressure to give a residue, the residue was purified by column chromatography (SiO₂, Petroleum ether/Ethyl acetate=20/1 to 3/1). Compound C (1.2 g, 5.82 mmol, 37.28% yield) was obtained as a white solid. LCMS: MS (ESI) Retention time: 0.596 min (M+1)+=207.1.

To a solution of Compound C (1.2 g, 5.82 mmol, 1 eq) in THF (15 mL) and H₂O (5 mL) was added Oxone (17.88 g, 29.09 mmol, 5 eq). The mixture was stirred at 25° C. for 12 hrs. LC-MS showed desired mass was detected. The reaction mixture was filtered to get a solution and concentrated under reduced pressure to remove THF, then was diluted with H₂O 50 mL and extracted with EA 300 mL (100 mL*3). The combined organic layers were washed with brine 200 mL (100 mL*2), dried over Na₂SO₄, filtered and concentrated under reduced pressure to give a residue. The residue was used to the next step without purification. Compound D (500 mg, crude) was obtained as a white solid. LCMS: MS (ESI) Retention time: 0.592 min (M+1)+=239.3. ¹H NMR (400 MHz, DMSO-d6) δ=8.95 (d, J=0.8 Hz, 1H), 8.63 (d, J=8.4 Hz, 1H), 8.14 (dd, J=1.6, 8.4 Hz, 1H), 3.68 (s, 3H).

To a solution of Compound D (500 mg, 2.10 mmol, 1 eq) in DMF (8 mL) was added K₂CO₃ (870.00 mg, 6.29 mmol, 3 eq) and Compound 4A (691.27 mg, 2.73 mmol, 1.3 eq). The mixture was stirred at 25° C. for 12 hrs. LC-MS showed desired mass was detected. The reaction mixture was filtered and concentrated under reduced pressure to give a solution, the solution was purified by prep-HPLC (column: Phenomenex Luna C18 150*25 mm*10 um; mobile phase: [water (FA)-ACN]; gradient: 32%-62% B over 10 min). Compound E (320 mg, 777.46 mol, 37.05% yield) was obtained as colorless oil. LCMS: MS (ESI) Retention time: 0.751 min (M+1)+=412.3. ¹H NMR (400 MHz, CHLOROFORM-d) δ=7.80 (d, J=1.2 Hz, 1H), 7.68 (d, J=8.0 Hz, 1H), 7.58 (br s, 1H), 7.33 (dd, J=1.6, 8.0 Hz, 1H), 5.23 (s, 2H), 4.66 (s, 2H), 4.14 (t, J=5.6 Hz, 2H), 3.54-3.45 (m, 2H), 2.91 (br t, J=5.2 Hz, 2H), 0.96-0.85 (m, 2H), −0.02 (s, 9H).

To a solution of Compound E (150 mg, 364.44 mol, 1 eq) in HCl/MeOH (5 mL). The mixture was stirred at 60° C. for 12 hrs. LC-MS (EW43310-133-P1B) showed desired mass was detected, the reaction mixture was concentrated under reduced pressure to give a residue. The crude product was triturated with MeCN for 5 min., then filtered and concentrated under reduced pressure to give a residue. The residue was used in the next step without purification. Compound 23 (80 mg, 239.21 mol, 65.64% yield, 94% purity) was obtained as a white solid. LCMS: MS (ESI) Retention time: 0.726 min (M+1)+=315.1. ¹H NMR (400 MHz, METHANOL-d₄) δ=8.88 (s, 1H), 8.18 (s, 1H), 7.90 (s, 2H), 4.98 (s, 2H), 4.14 (s, 2H), 3.94 (s, 3H), 3.07 (s, 2H).

To a solution of Compound 23 (20 mg, 63.62 mol, 1 eq) in THF (2 mL) was added 3, 4, 6, 7, 8, 9-hexahydro-2H-pyrimido[1, 2-a]pyrimidine (44.28 mg, 318.10 mol, 5 eq) and 2-methoxyethanamine (7.17 mg, 95.43 mol, 8.30 μL, 1.5 eq). The mixture was stirred at 50° C. for 12 hrs. LC-MS showed desired mass was detected. The reaction mixture was filtered and concentrated under reduced pressure to give a residue. The residue was purified by prep-HPLC (column: Phenomenex Luna C18 150*25 mm*10 um; mobile phase: [water (FA)-ACN]; gradient: 2%-32% B over 10 min). Compound 24 (7.92 mg, 20.83 mol, 32.74% yield, 94% purity) was obtained as an off-white solid. LCMS: MS (ESI) Retention time: 0.693 min (M+1)+=358.1. ¹H NMR (400 MHz, METHANOL-d₄) δ=7.93 (d, J=1.6 Hz, 1H), 7.77-7.71 (m, 2H), 7.55 (dd, J=1.6, 8.4 Hz, 1H), 4.70 (s, 2H), 4.01 (t, J=5.6 Hz, 2H), 3.58 (s, 4H), 3.39 (s, 3H), 2.88 (br t, J=5.6 Hz, 2H).

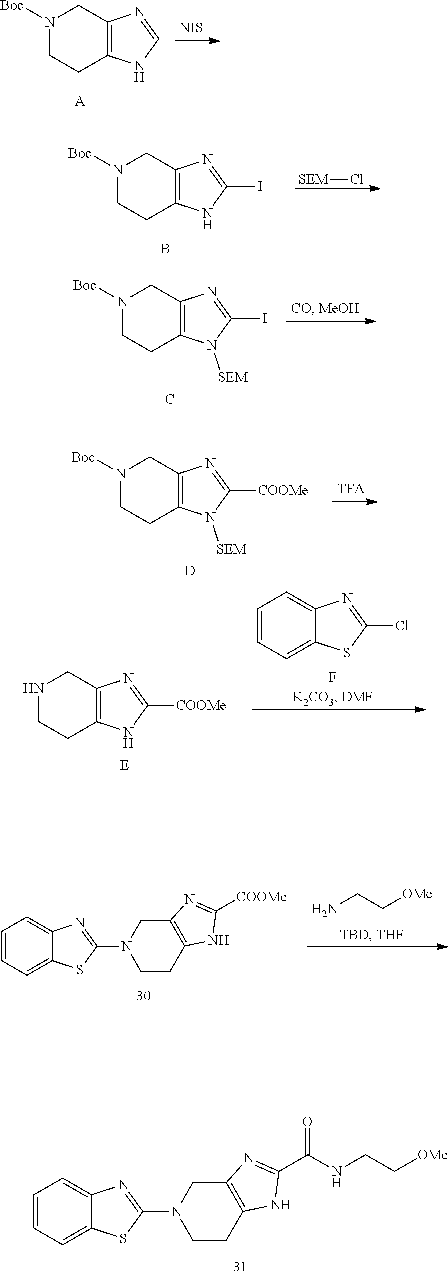

To a solution of Compound A (5 g, 22.39 mmol, 1 eq) in THF (40 mL) was added NIS (7.56 g, 33.59 mmol, 1.5 eq) under N₂. The mixture was stirred at 25° C. for 1 hr. LC-MS showed desired mass was detected. The reaction mixture was concentrated under reduced pressure to remove THF, then the mixture was quenched with sat. NaHCO₃20 mL, and diluted with H₂O 50 mL and extracted with EA 300 mL (100 mL*3). The combined organic layers were washed with brine 200 mL (100 mL*2), dried over Na₂SO₄, filtered and concentrated under reduced pressure to give a residue. The residue was purified by column chromatography (SiO₂, Petroleum ether/Ethyl acetate=20/1 to 1/2). Compound B (3 g, 8.59 mmol, 38.37% yield) was obtained as a yellow solid. ¹H NMR (400 MHz, CHLOROFORM-d) δ=4.51 (br s, 2H), 3.71 (br s, 2H), 2.72 (br t, J=5.2 Hz, 2H), 1.48 (s, 9H).

To a solution of Compound 2 (3 g, 8.59 mmol, 1 eq) in THF (60 mL) was added NaH (378.01 mg, 9.45 mmol, 60% purity, 1.1 eq). The mixture was stirred at 25° C. for 1.5 hr. Then 2-(chloromethoxy) ethyl-trimethyl-silane (1.58 g, 9.45 mmol, 1.67 mL, 1.1 eq) was added at 0° C., and the mixture was stirred at 25° C. for 5 hrs. LC-MS showed desired mass was detected. The reaction mixture was quenched by addition NH₄Cl 50 mL, and then diluted with H₂O 200 mL and extracted with EA 900 mL (300 mL*3). The combined organic layers were washed with brine 200 mL (100 mL*2), dried over Na₂SO₄, filtered and concentrated under reduced pressure to give a residue. The residue was purified by flash silica gel chromatography (column: Welch Ultimate XB-SiOH 250*70*10 um; mobile phase: [Hexane-EtOH]; gradient: 1%-15% B over 15 min.). Compound C (350 mg, 730.04 mol, 8.50% yield) was obtained as a yellow solid.

To a solution of Compound C (250 mg, 521.46 mol, 1 eq) in MeOH (30 mL) was added TEA (105.53 mg, 1.04 mmol, 145.16 μL, 2 eq) and Pd(PPh₃)₄ (120.51 mg, 104.29 mol, 0.2 eq). The mixture was stirred at 100° C. for 2 hr under CO (50 psi). LC-MS showed desired mass was detected. The reaction mixture was filtered and concentrated under reduced pressure to give a residue. The residue was purified by prep-HPLC (column: Phenomenex luna C18 150*25 mm*10 um; mobile phase: [water (FA)-ACN]; gradient: 56%-86% B over 10 min). Compound D (150 mg, 364.46 mol, 69.89% yield) was obtained as yellow oil. ¹H NMR (400 MHz, CHLOROFORM-d) δ=5.93-5.64 (m, 2H), 4.64-4.45 (m, 2H), 3.95 (s, 3H), 3.81-3.67 (m, 2H), 3.61-3.52 (m, 2H), 2.82-2.69 (m, 2H), 1.51-1.47 (m, 9H), 0.96-0.84 (m, 2H), −0.02 (d, J=4.0 Hz, 9H).

To a solution of Compound D (100 mg, 242.97 mol, 1 eq) in DCM (0.6 mL) was added TFA (307.00 mg, 2.69 mmol, 0.2 mL, 11.08 eq). The mixture was stirred at 25° C. for 4 hrs. LC-MS showed desired mass was detected. The reaction mixture was filtered and concentrated under reduced pressure to give a residue. The residue was purified by prep-HPLC (column: Phenomenex luna C18 150*25 mm*10 um; mobile phase: [water (FA)-ACN]; gradient: 0%-6% B over 6 min). Compound E (30 mg, 165.57 mol, 68.14% yield) was obtained as a white solid.

To a solution of Compound E (30 mg, 165.57 mol, 1 eq) in DMF (0.5 mL) was added K₂CO₃ (68.65 mg, 496.71 mol, 3 eq) and Compound F (33.70 mg, 198.68 mol, 25.87 μL, 1.2 eq). The mixture was stirred at 40° C. for 12 hrs. LC-MS showed desired mass was detected. The reaction mixture was filtered and concentrated under reduced pressure to give a solution. The solution was purified by prep-HPLC (column: Phenomenex luna C18 150*25 mm*10 um; mobile phase: [water (FA)-ACN]; gradient: 26%-46% B over 9 min). Compound 30 (1.76 mg, 5.43 mol, 4.92% yield, 97% purity) was obtained as an off-white solid. LCMS: MS (ESI) Retention time: 0.718 min, (M+1)+=315.1. ¹H NMR (400 MHz, METHANOL-d₄) δ=7.68 (d, J=7.6 Hz, 1H), 7.51 (d, J=8.0 Hz, 1H), 7.31 (t, J=8.0 Hz, 1H), 7.11 (t, J=7.6 Hz, 1H), 4.72 (s, 2H), 4.03 (t, J=5.6 Hz, 2H), 3.92 (s, 3H), 2.93 (t, J=6.0 Hz, 2H).

To a solution of Compound 30 (10 mg, 31.81 mol, 1 eq) in THF (4 mL) was added 3, 4, 6, 7, 8, 9-hexahydro-2H-pyrimido[1, 2-a]pyrimidine (22.14 mg, 159.05 mol, 5 eq) and 2-methoxyethanamine (2.87 mg, 38.17 mol, 3.32 μL, 1.2 eq). The mixture was stirred at 25° C. for 12 hrs. The reaction mixture was filtered and concentrated under reduced pressure to give a residue. The residue was purified by prep-HPLC (column: Phenomenex luna C18 150*25 mm*10 um; mobile phase: [water (FA)-ACN]; gradient: 23%-53% B over 10 min). Compound 31 (2.81 mg, 7.67 mol, 24.12% yield, 97.6% purity) was obtained as a white solid. LCMS: MS (ESI) Retention time: 0.776 min, (M+1)+=358.2. ¹H NMR (400 MHz, METHANOL-d₄) δ=7.68 (d, J=8.0 Hz, 1H), 7.51 (d, J=8.0 Hz, 1H), 7.33-7.28 (m, 1H), 7.14-7.07 (m, 1H), 4.77-4.62 (m, 2H), 4.02 (br t, J=5.2 Hz, 2H), 3.56 (s, 4H), 3.39 (s, 3H), 2.92 (br s, 2H).

### Testing of Compounds for the Treatment of AP-4 Deficiency

SH-SY5Y cell culture. AP4B1 wildtype (AP4B1^{WT}) and AP4B1 knockout (AP4B1KO) SH-SY5Y cells were generated previously (PMID: 38233389, PMID: 35217685). Undifferentiated SH-SY5Y cells were maintained in DMEM/F12 (Gibco, Cat #11320033) supplemented with 10% heat-inactivated fetal bovine serum (Gibco, Cat #10438026), 100 U/mL penicillin and 100 #g/mL streptomycin at 37° C. under 5% C₀₂. SH-SY5Y cells were passaged every 2-3 days and differentiated into a neuron-like state using a 5-day differentiation protocol with all-trans-retinoic acid (MedChemExpress, #HY-14649). For assessment of ATG9A translocation, differentiated SH-SY5Y cells were plated in 96-well plates (Greiner Bio-One, Cat #655090) at a density of 1×10⁴ cells per well. Media changes were done every 2-3 days and drugs were administered 24 h before fixation.

Immunocytochemistry. The immunocytochemistry workflow was optimized for high-throughput using automated pipettes and reagent dispensers (Thermo Fisher Scientific Multidrop Combi Reagent Dispenser, Integra VIAFLO 96/384 liquid handler, Integra VOYAGER pipette). SH-SY5Y cells were fixed using 4% PFA, permeabilized with 0.1% saponin in PBS and blocked in 1% BSA/0.01% saponin (blocking solution) in PBS. Primary antibody (diluted in blocking solution) was added for 1 h at room temperature. Plates were gently washed three times in blocking solution, followed by addition of fluorochrome-conjugated secondary antibodies and Hoechst 33258 for 30 min at room temperature. Plates were then gently washed three times with PBS and protected from light.

High-content imaging and automated image analysis. High-throughput confocal imaging was performed on the ImageX-press Micro Confocal Screening System (Molecular Devices) using an experimental pipeline modified from the pipeline described in Behne et al. (PMID: 31915823). Up to 36 fields were acquired in a 6×6 format (96-well plate) using a 40×S Plan Fluor objective (NA 0.60 m, WB 3.6-2.8 mm). Image analysis was performed using a customized image analysis pipeline in MetaXpress (Molecular Devices): Briefly, cells were identified based on the presence of DAPI signal inside a TUBB3-positive cell body. Sequential masks were generated for (1) the TGN by outlining the area covered by TGN marker TGN46 (TGN46-positive area) and (2) for the cell area outside the TGN (TUBB3-positive area minus TGN46-positive area). ATG9A fluorescence intensity (F.U.) was measured in both compartments in each cell, and the ATG9A ratio was calculated by dividing the ATG9A fluorescence intensity inside the TGN by the ATG9A fluorescence intensity in the remaining cell body:

$$ATG9A Ratio = \frac{ATG9AF . U . inside the TGN}{ATG9AF . U . outside the TGN}$$

Z′-factor robust values and strictly standardized median difference (SSMD) were calculated for each plate and only plates that met the predefined quality metrics of a Z′-factor robust 0.3 and SSMD 3 were included in analyses.

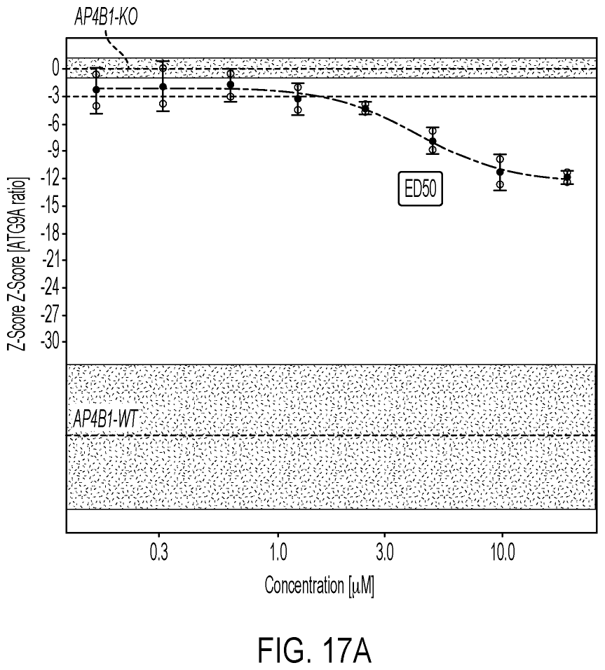

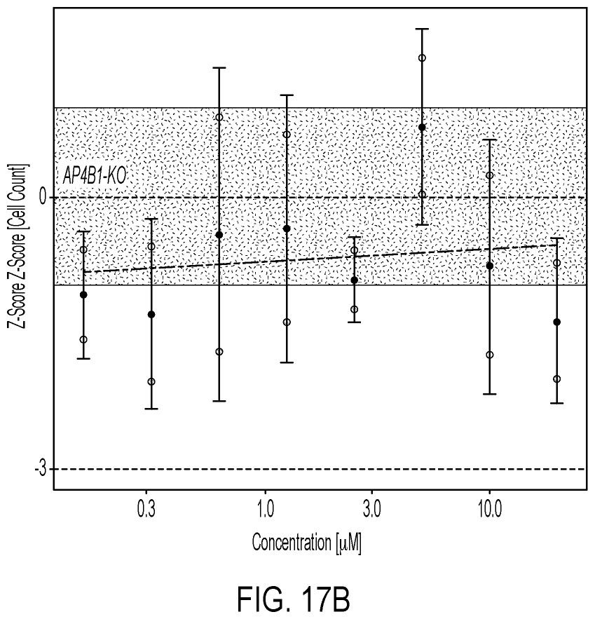

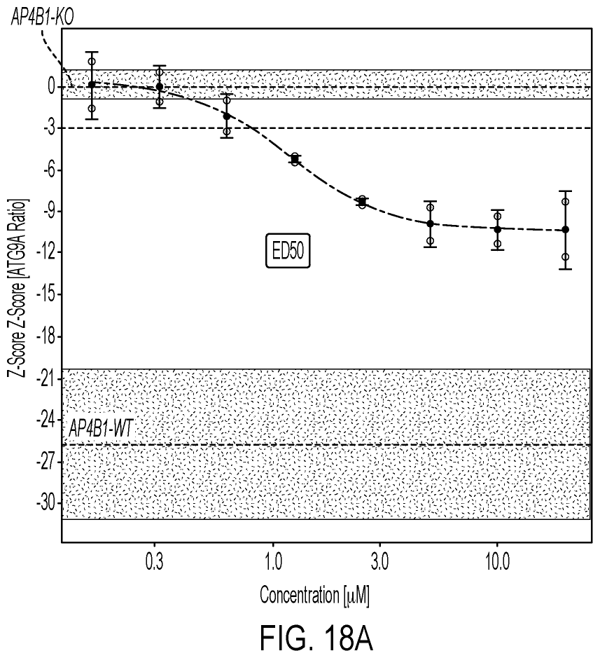

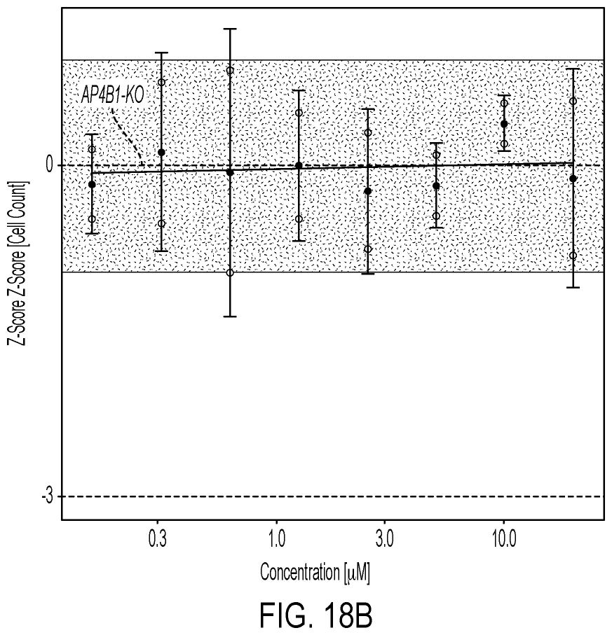

The results in Table 1 show that compounds of the disclosure are effective in treating AP-4 deficiency. FIGS. 17A and 18A show dose response curves for Compounds 10 and 17.

TABLE 1

| | | | Z atg9a | | Z atg9a |
|---|---|---|---|---|---|
| | | EC50 (z | ratio | | ratio |
| Cell | | atg9a | Std | EC50 (atg9a | Std |
| type | compound | ratio) | error | translocation) | error |
| AP4B | AP4B1-KO + | 1.61847 | 0.55306 | 1.71467 | 0.39518 |
| 1-KO | Compound 1 | | | | |
| AP4B | AP4B1-KO + | 4.418 | 1.0028 | 4.2261 | 1.3495 |
| 1-KO | Compound 10 | | | | |
| AP4B | AP4B1-KO + | 1.18463 | 0.24406 | 1.16752 | 0.2571 |
| 1-KO | Compound 17 | | | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I): 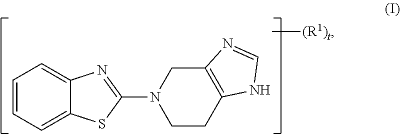
or a pharmaceutically acceptable salt thereof, wherein:
each occurrence of R¹ is, independently, halogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, —OR^{A}, —N(R^{A})₂, —SR^{A}, —CN, —SCN, —C(═NR^{A})R^{A}, —C(═NR^{A})OR^{A}, —C(═NR^{A})N(R^{A})₂, —C(═O)R^{A}, —C(═O)OR^{A}, —C(═O)N(R^{A})₂, —C(═O)NR^{A}S(O)₂R^{A}, —NO₂, —NR^{A}C(═O)R^{A}, —NR^{A}C(═O)OR^{A}, —NR^{A}C(═O)N(R^{A})₂, —NR^{A}C(═NR^{A})N(R^{A})₂, —OC(═O)R^{A}, —OC(═O)OR^{A}, —OC(═O)N(R^{A})₂, —NR^{A}S(O)₂R^{A}, OS(O)₂R^{A}, —S(O)₂NR^{A}C(O)R^{A}, —S(O)₂N(R^{A})₂, —S(O)₂OR^{A} or —S(O)₂R^{A}; or two R¹ groups are joined to form a substituted or unsubstituted carbocyclyl ring, a substituted or unsubstituted aryl ring, a substituted or unsubstituted heterocyclyl ring, or a substituted or unsubstituted heteroaryl ring;
t is 0 or a positive integer; and
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroaliphatic, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two R^{A} groups are joined to form a substituted or unsubstituted heterocyclyl ring, or a substituted or unsubstituted heteroaryl ring;
wherein, when present, each occurrence of R¹ is bound to any substitutable atom of the compound.

2. The compound of claim 1, wherein the compound is of Formula (I-a): 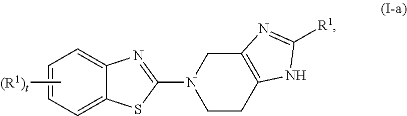
or a pharmaceutically acceptable salt thereof.

3. The compound of claim 1 or 2, wherein the compound is of Formula (I-b): 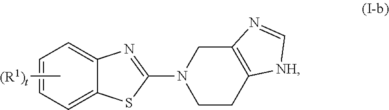
or a pharmaceutically acceptable salt thereof.

4. The compound of any one of claims 1-3, wherein the compound is of Formula (I-c): 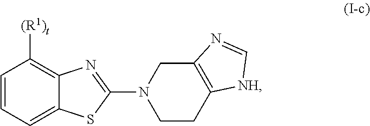
or a pharmaceutically acceptable salt thereof.

5. The compound of any one of claims 1-4, wherein the compound is of Formula (I-d): 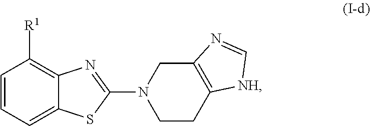
or a pharmaceutically acceptable salt thereof.

6. The compound of any one of claims 1-5, or a pharmaceutically acceptable salt thereof, wherein:
each occurrence of R¹ is, independently, halogen, substituted or unsubstituted acyl, substituted or unsubstituted alkyl, substituted or unsubstituted alkenyl, substituted or unsubstituted alkynyl, substituted or unsubstituted heteroaliphatic, —OR^{A}, —N(R^{A})₂, —SR^{A}, —CN, —SCN, —C(═O)R^{A}, —C(═O)OR^{A}, —C(═O)N(R^{A})₂, —C(═O)NR^{A}S(O)₂R^{A}, —S(O)₂NR^{A}C(O)R^{A}, —S(O)₂N(R^{A})₂, —S(O)₂OR^{A} or —S(O)₂R^{A}.

7. The compound of any one of claims 1-6, or a pharmaceutically acceptable salt thereof, wherein:
each occurrence of R¹ is, independently, substituted or unsubstituted acyl, substituted or unsubstituted alkyl, substituted or unsubstituted heteroalkyl, —OR^{A}, —C(═O)OR^{A}, or —C(═O)N(R^{A})₂.

8. The compound of any one of claims 1-7, or a pharmaceutically acceptable salt thereof, wherein:
each occurrence of R¹ is, independently, substituted or unsubstituted alkyl, —OR^{A}, —C(═O)OR^{A}, or —C(═O)N(R^{A})₂.

9. The compound of any one of claims 1-8, or a pharmaceutically acceptable salt thereof, wherein:
each occurrence of R¹ is, independently, substituted or unsubstituted alkyl, —OR^{A}, —C(═O)OR^{A}, or —C(═O)N(R^{A})₂; wherein each occurrence of R^{A} is, independently, hydrogen, substituted or unsubstituted alkyl, or substituted or unsubstituted heteroalkyl.

10. The compound of any one of claims 1-9, or a pharmaceutically acceptable salt thereof, wherein:
each occurrence of R¹ is, independently, unsubstituted alkyl, —OR^{A}, —C(═O)OR^{A}, or —C(═O)N(R^{A})₂; wherein each occurrence of R^{A} is, independently, hydrogen, substituted or unsubstituted alkyl, or substituted or unsubstituted heteroalkyl.

11. The compound of any one of claims 1-10, or a pharmaceutically acceptable salt thereof, wherein:
each occurrence of R¹ is, independently, unsubstituted C₁₋₄alkyl, —OH, —OC₁₋₄alkyl, —C(═O)OC₁₋₄alkyl, or —C(═O)NH—(C₁₋₄alkylene)-OC₁₋₄alkyl.

12. The compound of any one of claims 1-11, or a pharmaceutically acceptable salt thereof, wherein:
each occurrence of R¹ is, independently, —CH₃, —OH, —OCH₃, —C(═O)OCH₃, or —C(═O)NH(CH₂CH₂)OCH₃.

13. The compound of any one of claims 1-12, or a pharmaceutically acceptable salt thereof, wherein:
t is 0 or 1.

14. The compound of any one of claims 1-13, or a pharmaceutically acceptable salt thereof, wherein:
t is 0.

15. The compound of any one of claims 1-13, or a pharmaceutically acceptable salt thereof, wherein:
t is 1.

16. The compound of claim 1, or a pharmaceutically acceptable salt thereof, wherein the compound is not of the formula:

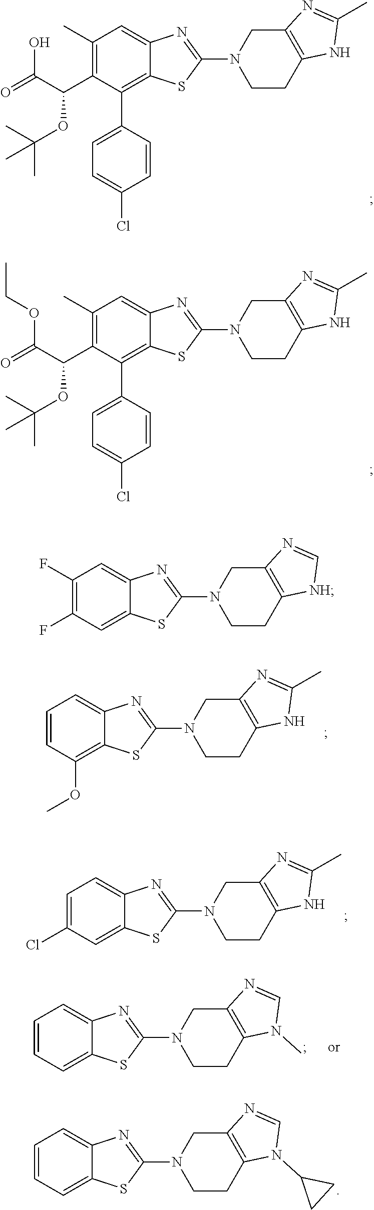

17. The compound of claim 1, wherein the compound is of formula: 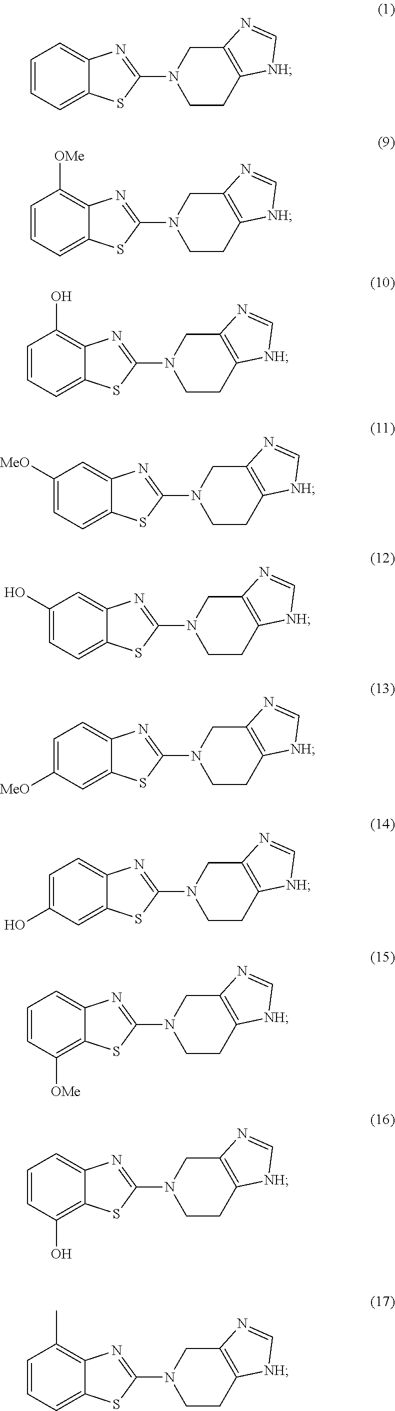 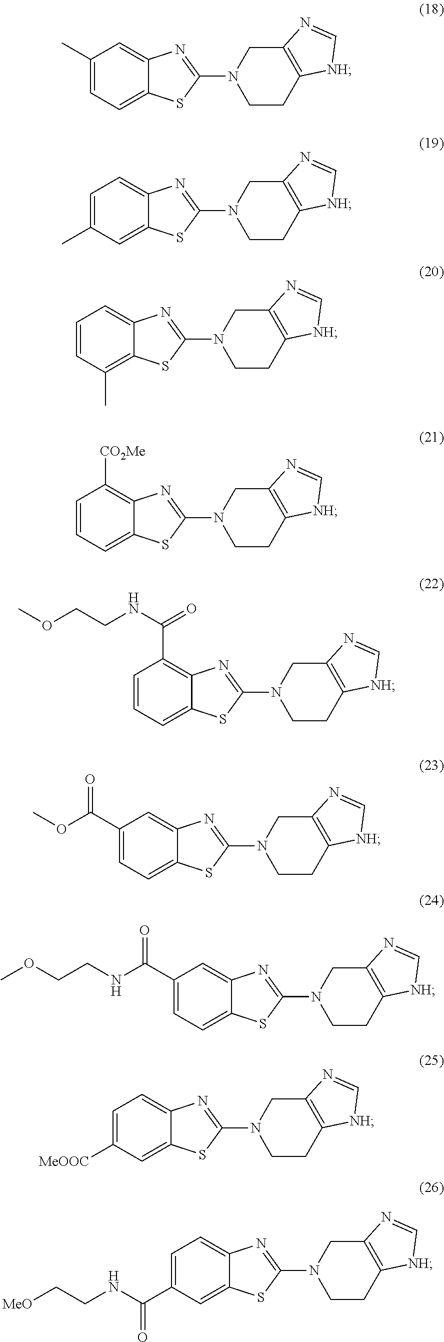 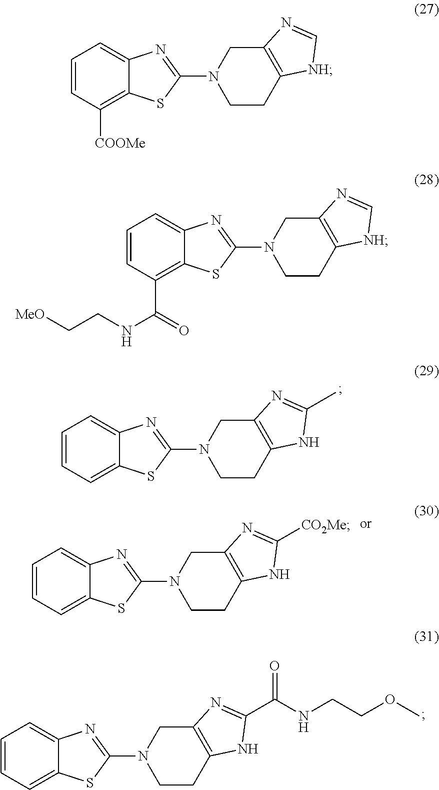 [SMILES: COC(=O)c1cccc2nc(N3CCc4[nH]cnc4C3)sc12 ; COCCNC(=O)c1cccc2nc(N3CCc4[nH]cnc4C3)sc12 ; Cc1nc2c([nH]1)CCN(c1nc3ccccc3s1)C2 ; COC(=O)c1nc2c([nH]1)CCN(c1nc3ccccc3s1)C2 ; "or" ; COCCNC(=O)c1nc2c([nH]1)CCN(c1nc3ccccc3s1)C2]
or a pharmaceutically acceptable salt.

18. The compound of claim 1, wherein the compound is of formula: 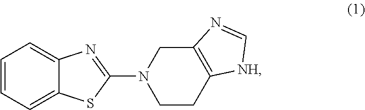 [SMILES: c1ccc2sc(N3CCc4[nH]cnc4C3)nc2c1]
or a pharmaceutically acceptable salt.

19. The compound of claim 1, wherein the compound is of formula: 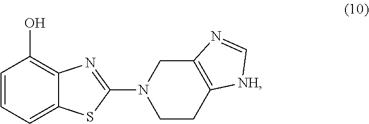 [SMILES: Oc1cccc2sc(N3CCc4[nH]cnc4C3)nc12]
or a pharmaceutically acceptable salt.

20. The compound of claim 1, wherein the compound is of formula: 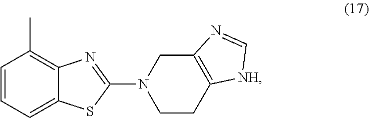 [SMILES: Cc1cccc2sc(N3CCc4[nH]cnc4C3)nc12]
or a pharmaceutically acceptable salt.

21. A pharmaceutical composition comprising a compound of any of claims 1-20, or a pharmaceutically acceptable salt thereof, and a pharmaceutically acceptable excipient.

22. A kit comprising a compound of any of claims 1-20, or a pharmaceutically acceptable salt thereof, or a pharmaceutical composition of claim 21, and instructions for administering the compound or pharmaceutical composition to a subject in need thereof.

23. A method of treating a neurological disease or disorder, the method comprising administering an effective amount of a compound of any of claims 1-20, or a pharmaceutically acceptable salt thereof, or a pharmaceutical composition of claim 21, to a subject in need thereof.

24. The method of claim 23, wherein the neurological disease or disorder is a hereditary spastic paraplegia (HSP).

25. The method of claim 23 or 24, wherein the neurological disease or disorder is Adaptor protein complex 4 (AP-4)-related hereditary spastic paraplegia (AP-4-HSP).

26. A method of modulating Autophagy Related 9A (ATG9A) trafficking in or from a cell, the method comprising contacting an effective amount of a compound of any of claims 1-20, or a pharmaceutically acceptable salt thereof, or a pharmaceutical composition of claim 21, with the cell.

27. The method of claim 26, wherein the contacting results in an increase in trafficking of ATG9A out of the trans-Golgi network (TGN).

28. The method of claim 26 or 27, wherein the contacting results in a decrease of ATG9A in the trans-Golgi network (TGN).

29. The method of any one of claims 26-28, wherein the contacting results in a decrease of the ratio of the concentration of ATG9A in the trans-Golgi network (TGN) to the concentration of ATG9A in the cytoplasm.

30. A method of modulating intracellular vesicle trafficking and increasing autophagic flux in a cell, the method comprising contacting an effective amount of a compound of any of claims 1-20, or a pharmaceutically acceptable salt thereof, or a pharmaceutical composition of claim 21, with the cell.

31. The method of any of claims 26-30, wherein the contacting is in vitro.

32. The method of any of claims 26-30, wherein the contacting is in vivo.

33. The method of any of claims 26-32, wherein the cell is a mammalian cell.

34. The method of any of claims 26-33, wherein the cell is a human cell.
